SEQUENCE LISTING (129 sequences)

SEQ ID NO: 1 - DNA, 23 bp, unidentified
  source 1..23: mol_type = other DNA
ctacgtaaagaagtcggcatggt

SEQ ID NO: 2 - DNA, 24 bp, unidentified
  source 1..24: mol_type = other DNA
aagccacattgtcatatacggaaa

SEQ ID NO: 3 - DNA, 22 bp, unidentified
  source 1..22: mol_type = other DNA
tcaacagccaaccccattcccg

SEQ ID NO: 4 - DNA, 23 bp, unidentified
  source 1..23: mol_type = other DNA
taatgtagtttatggtttgcgtc

SEQ ID NO: 5 - DNA, 19 bp, unidentified
  source 1..19: mol_type = other DNA
ctcatcccataaagcagcc

SEQ ID NO: 6 - DNA, 30 bp, unidentified
  source 1..30: mol_type = other DNA
agatattttagatcaaagagtggaagaaag

SEQ ID NO: 7 - DNA, 20 bp, unidentified
  source 1..20: mol_type = other DNA
tgggtcaagcaagaacattg

SEQ ID NO: 8 - DNA, 25 bp, unidentified
  source 1..25: mol_type = other DNA
cttagctactaatgattccaattga

SEQ ID NO: 9 - DNA, 31 bp, unidentified
  source 1..31: mol_type = other DNA
acagtaattccgttcttaactacttttttag

SEQ ID NO: 10 - DNA, 24 bp, unidentified
  source 1..24: mol_type = other DNA
gatggaagttgctattttggaaaa

SEQ ID NO: 11 - DNA, 21 bp, unidentified
  source 1..21: mol_type = other DNA
tggtcgtgacttacctaaacg

SEQ ID NO: 12 - DNA, 25 bp, unidentified
  source 1..25: mol_type = other DNA
aacgcctctagaagaagcacaagcg

SEQ ID NO: 13 - DNA, 23 bp, unidentified
  source 1..23: mol_type = other DNA
cgaggttaataccggatactcca

SEQ ID NO: 14 - DNA, 18 bp, unidentified
  source 1..18: mol_type = other DNA
tctcaacccggctaccca

SEQ ID NO: 15 - DNA, 19 bp, unidentified
  source 1..19: mol_type = other DNA
tcctttgccgccggagctt

SEQ ID NO: 16 - DNA, 20 bp, unidentified
  source 1..20: mol_type = other DNA
cgatcagtaggggttctgag

SEQ ID NO: 17 - DNA, 19 bp, unidentified
  source 1..19: mol_type = other DNA
tacttggctggttcaggct

SEQ ID NO: 18 - DNA, 22 bp, unidentified
  source 1..22: mol_type = other DNA
aggagtttggaccgtgtctcag

SEQ ID NO: 19 - DNA, 19 bp, unidentified
  source 1..19: mol_type = other DNA
tgcagcaactaacatgacc

SEQ ID NO: 20 - DNA, 22 bp, unidentified
  source 1..22: mol_type = other DNA
ttaatcgtttgactaacacccc

SEQ ID NO: 21 - DNA, 20 bp, unidentified
  source 1..20: mol_type = other DNA
ctgcgaagaagctgttccct

SEQ ID NO: 22 - DNA, 21 bp, unidentified
  source 1..21: mol_type = other DNA
ggctcgctttggatactagca

SEQ ID NO: 23 - DNA, 19 bp, unidentified
  source 1..19: mol_type = other DNA
ctttcgtccctcagcgtca

SEQ ID NO: 24 - DNA, 21 bp, unidentified
  source 1..21: mol_type = other DNA
ccagtaagctgccttcgcctt

SEQ ID NO: 25 - DNA, 19 bp, unidentified
  source 1..19: mol_type = other DNA
gcaactatttctgcagcag

SEQ ID NO: 26 - DNA, 21 bp, unidentified
  source 1..21: mol_type = other DNA
ttgtcttcaacggtaacaaca

SEQ ID NO: 27 - DNA, 25 bp, unidentified
  source 1..25: mol_type = other DNA
cctgaccaaccttatccaaagcttc

SEQ ID NO: 28 - DNA, 20 bp, unidentified
  source 1..20: mol_type = other DNA
atggataggggttctgagag

SEQ ID NO: 29 - DNA, 19 bp, unidentified
  source 1..19: mol_type = other DNA
atattcctcactgctgcct

SEQ ID NO: 30 - DNA, 22 bp, unidentified
  source 1..22: mol_type = other DNA
ccacattggdactgagacacgg

SEQ ID NO: 31 - DNA, 20 bp, unidentified
  source 1..20: mol_type = other DNA
cagcagcttaactgttggtc

SEQ ID NO: 32 - DNA, 19 bp, unidentified
  source 1..19: mol_type = other DNA
tcctgatatctgcgcattc

SEQ ID NO: 33 - DNA, 22 bp, unidentified
  source 1..22: mol_type = other DNA
agtcacccctaccgcactcaag

SEQ ID NO: 34 - DNA, 21 bp, unidentified
  source 1..21: mol_type = other DNA
tagacccgacaagagacagga

SEQ ID NO: 35 - DNA, 17 bp, unidentified
  source 1..17: mol_type = other DNA
cgggaagggtggatggt

SEQ ID NO: 36 - DNA, 20 bp, unidentified
  source 1..20: mol_type = other DNA
catcctccgcgggggtataa

SEQ ID NO: 37 - DNA, 18 bp, synthetic construct
  source 1..18: mol_type = other DNA
cagcaacagcagcaagaa

SEQ ID NO: 38 - DNA, 20 bp, unidentified
  source 1..20: mol_type = other DNA
ctggcattgacatctgtttg

SEQ ID NO: 39 - DNA, 27 bp, unidentified
  source 1..27: mol_type = other DNA
agatgctgaaggacaacaacaacaaca

SEQ ID NO: 40 - DNA, 25 bp, unidentified
  source 1..25: mol_type = other DNA
cgaaactccaaartaccacgttacc

SEQ ID NO: 41 - DNA, 25 bp, unidentified
  source 1..25: mol_type = other DNA
caccagcaataatcaaratagcaca

SEQ ID NO: 42 - DNA, 28 bp, unidentified
  source 1..28: mol_type = other DNA
tgctccaggtcacagagatttcatcaag

SEQ ID NO: 43 - DNA, 20 bp, unidentified
  source 1..20: mol_type = other DNA
atgtcgcgcaagactgtaac

SEQ ID NO: 44 - DNA, 20 bp, unidentified
  source 1..20: mol_type = other DNA
cagttgcaaccacctgttga

SEQ ID NO: 45 - DNA, 20 bp, unidentified
  source 1..20: mol_type = other DNA
tggccaccacctgccagtca

SEQ ID NO: 46 - DNA, 24 bp, unidentified
  source 1..24: mol_type = other DNA
cttatagggatggctatcagtaat

SEQ ID NO: 47 - DNA, 20 bp, unidentified
  source 1..20: mol_type = other DNA
ccgttatctgtttgtgatgc

SEQ ID NO: 48 - DNA, 27 bp, unidentified
  source 1..27: mol_type = other DNA
tgctgagctacttagacttgaaactac

SEQ ID NO: 49 - DNA, 21 bp, unidentified
  source 1..21: mol_type = other DNA
agtcagtgatttcggctcgtt

SEQ ID NO: 50 - DNA, 21 bp, unidentified
  source 1..21: mol_type = other DNA
gcctgccaagccgataaagga

SEQ ID NO: 51 - DNA, 20 bp, unidentified
  source 1..20: mol_type = other DNA
agctgccaaacagccttcag

SEQ ID NO: 52 - DNA, 19 bp, unidentified
  source 1..19: mol_type = other DNA
caatgaaaatccattgcgt

SEQ ID NO: 53 - DNA, 19 bp, unidentified
  source 1..19: mol_type = other DNA
tctgactgtgagaatatat

SEQ ID NO: 54 - DNA, 21 bp, unidentified
  source 1..21: mol_type = other DNA
tagaggccggtctatttatga

SEQ ID NO: 55 - DNA, 22 bp, unidentified
  source 1..22: mol_type = other DNA
gtgcaaaaccttactatgttgt

SEQ ID NO: 56 - DNA, 21 bp, unidentified
  source 1..21: mol_type = other DNA
tgaacgtccatatgttccaaa

SEQ ID NO: 57 - DNA, 24 bp, unidentified
  source 1..24: mol_type = other DNA
tgggtgtaattgctcgttgtgtag

SEQ ID NO: 58 - DNA, 21 bp, unidentified
  source 1..21: mol_type = other DNA
caacagccttccgtgaactct

SEQ ID NO: 59 - DNA, 19 bp, unidentified
  source 1..19: mol_type = other DNA
cgaggccttcgttgacgta

SEQ ID NO: 60 - DNA, 25 bp, unidentified
  source 1..25: mol_type = other DNA
ccagaagatgtacgctcgccgtgct

SEQ ID NO: 61 - DNA, 22 bp, unidentified
  source 1..22: mol_type = other DNA
gaattcyactattttggaggac

SEQ ID NO: 62 - DNA, 19 bp, unidentified
  source 1..19: mol_type = other DNA
ttgacaagcaattgcctgg

SEQ ID NO: 63 - DNA, 23 bp, unidentified
  source 1..23: mol_type = other DNA
ccaggaggcacactagaagatac

SEQ ID NO: 64 - DNA, 20 bp, unidentified
  source 1..20: mol_type = other DNA
tgacactgtgcctcaatcct

SEQ ID NO: 65 - DNA, 22 bp, unidentified
  source 1..22: mol_type = other DNA
acaactgggagtcagaggtaac

SEQ ID NO: 66 - DNA, 24 bp, unidentified
  source 1..24: mol_type = other DNA
aggtgaagcacgcatacctgtgcc

SEQ ID NO: 67 - DNA, 19 bp, unidentified
  source 1..19: mol_type = other DNA
attacccgacagctcagat

SEQ ID NO: 68 - DNA, 17 bp, unidentified
  source 1..17: mol_type = other DNA
ccgaggcaactgaccta

SEQ ID NO: 69 - DNA, 20 bp, unidentified
  source 1..20: mol_type = other DNA
ctgagattgacctgcaatgc

SEQ ID NO: 70 - DNA, 21 bp, unidentified
  source 1..21: mol_type = other DNA
tacartgcaatgagcaattgg

SEQ ID NO: 71 - DNA, 23 bp, unidentified
  source 1..23: mol_type = other DNA
ccaccktgcaggaaattgtgtta

SEQ ID NO: 72 - DNA, 22 bp, unidentified
  source 1..22: mol_type = other DNA
tttataaccctgatacacaacg

SEQ ID NO: 73 - DNA, 19 bp, unidentified
  source 1..19: mol_type = other DNA
aaatagcgggacgytggac

SEQ ID NO: 74 - DNA, 22 bp, unidentified
  source 1..22: mol_type = other DNA
caacagggacaatgtttcagtt

SEQ ID NO: 75 - DNA, 23 bp, unidentified
  source 1..23: mol_type = other DNA
cctgataaggagcgcctagtatg

SEQ ID NO: 76 - DNA, 19 bp, unidentified
  source 1..19: mol_type = other DNA
gccatcccatactcctatg

SEQ ID NO: 77 - DNA, 24 bp, unidentified
  source 1..24: mol_type = other DNA
gaaaccagtaacaaatatgctggt

SEQ ID NO: 78 - DNA, 21 bp, unidentified
  source 1..21: mol_type = other DNA
ctcatgtagcatctgctgttg

SEQ ID NO: 79 - DNA, 21 bp, unidentified
  source 1..21: mol_type = other DNA
cactgacaacaaaaggtaacg

SEQ ID NO: 80 - DNA, 20 bp, unidentified
  source 1..20: mol_type = other DNA
gtagtgtcgcctyacattta

SEQ ID NO: 81 - DNA, 17 bp, unidentified
  source 1..17: mol_type = other DNA
gtctagcttgctgtggc

SEQ ID NO: 82 - DNA, 22 bp, unidentified
  source 1..22: mol_type = other DNA
taactgcatggtcgggttcatc

SEQ ID NO: 83 - DNA, 19 bp, unidentified
  source 1..19: mol_type = other DNA
ggatgaccacttatgccaa

SEQ ID NO: 84 - DNA, 22 bp, unidentified
  source 1..22: mol_type = other DNA
tgctcgtagcattgcaagtcaa

SEQ ID NO: 85 - DNA, 17 bp, unidentified
  source 1..17: mol_type = other DNA
ccacgttgtgtctgtgt

SEQ ID NO: 86 - DNA, 25 bp, unidentified
  source 1..25: mol_type = other DNA
tgccactagtacctatacctaaggg

SEQ ID NO: 87 - DNA, 19 bp, unidentified
  source 1..19: mol_type = other DNA
ttgtgtgccatgaatacct

SEQ ID NO: 88 - DNA, 22 bp, unidentified
  source 1..22: mol_type = other DNA
aggaggtttgcatcctaaaatg

SEQ ID NO: 89 - DNA, 18 bp, unidentified
  source 1..18: mol_type = other DNA
taggcttacaagcagtgc

SEQ ID NO: 90 - DNA, 21 bp, unidentified
  source 1..21: mol_type = other DNA
tccagctggacaagcaaaacc

SEQ ID NO: 91 - DNA, 27 bp, unidentified
  source 1..27: mol_type = other DNA
catcctcatcctcatcctctgagctgt

SEQ ID NO: 92 - DNA, 26 bp, unidentified
  source 1..26: mol_type = other DNA
ctcgtgacatacaaggtcaaccggct

SEQ ID NO: 93 - DNA, 25 bp, unidentified
  source 1..25: mol_type = other DNA
cctatttcaaggcctacacatgccc

SEQ ID NO: 94 - DNA, 26 bp, unidentified
  source 1..26: mol_type = other DNA
aattgtggtacattaccacgcatggc

SEQ ID NO: 95 - DNA, 29 bp, unidentified
  source 1..29: mol_type = other DNA
taggttgtacaccgcccttgggagagcac

SEQ ID NO: 96 - DNA, 26 bp, unidentified
  source 1..26: mol_type = other DNA
cgtccaacctccacaccagaacaggc

SEQ ID NO: 97 - DNA, 23 bp, unidentified
  source 1..23: mol_type = other DNA
catctcctaccaatgctkccacc

SEQ ID NO: 98 - DNA, 23 bp, unidentified
  source 1..23: mol_type = other DNA
cattcyctattatctataccagg

SEQ ID NO: 99 - DNA, 22 bp, unidentified
  source 1..22: mol_type = other DNA
ctccaatggcaggtacacagcc

SEQ ID NO: 100 - DNA, 21 bp, unidentified
  source 1..21: mol_type = other DNA
tagaagccttaacagatgtga

SEQ ID NO: 101 - DNA, 22 bp, unidentified
  source 1..22: mol_type = other DNA
gttctaatgcctttacatcgtt

SEQ ID NO: 102 - DNA, 24 bp, unidentified
  source 1..24: mol_type = other DNA
aagcaaatggatctaaaatgcgaa

SEQ ID NO: 103 - DNA, 21 bp, unidentified
  source 1..21: mol_type = other DNA
ctgaatatgctatcgccggaa

SEQ ID NO: 104 - DNA, 17 bp, unidentified
  source 1..17: mol_type = other DNA
aatgtggccgttcatcc

SEQ ID NO: 105 - DNA, 25 bp, unidentified
  source 1..25: mol_type = other DNA
cagaccggctactgatcattgcctt

SEQ ID NO: 106 - DNA, 26 bp, unidentified
  source 1..26: mol_type = other DNA
atgcgtagatattaggaggaacaccg

SEQ ID NO: 107 - DNA, 20 bp, unidentified
  source 1..20: mol_type = other DNA
gggtcgatacctcctacayc

SEQ ID NO: 108 - DNA, 26 bp, unidentified
  source 1..26: mol_type = other DNA
accctggtagtccacgccgtaaacga

SEQ ID NO: 109 - DNA, 23 bp, unidentified
  source 1..23: mol_type = other DNA
ggtacttcccggttcgttgtatc

SEQ ID NO: 110 - DNA, 20 bp, unidentified
  source 1..20: mol_type = other DNA
acccaaacgcagcttgaatc

SEQ ID NO: 111 - DNA, 22 bp, unidentified
  source 1..22: mol_type = other DNA
cgatgatcgcagtgctgcccac

SEQ ID NO: 112 - DNA, 18 bp, unidentified
  source 1..18: mol_type = other DNA
ggcctggctatccggaga

SEQ ID NO: 113 - DNA, 17 bp, unidentified
  source 1..17: mol_type = other DNA
gcgcagagacatcgcga

SEQ ID NO: 114 - DNA, 16 bp, unidentified
  source 1..16: mol_type = other DNA
acgacttggcgttctg

SEQ ID NO: 115 - DNA, 22 bp, unidentified
  source 1..22: mol_type = other DNA
ttcttcggaacatcggtgacag

SEQ ID NO: 116 - DNA, 21 bp, unidentified
  source 1..21: mol_type = other DNA
ttgtaccagccattgtagcac

SEQ ID NO: 117 - DNA, 27 bp, unidentified
  source 1..27: mol_type = other DNA
cacgagctgacgacaaccatgcaccac

SEQ ID NO: 118 - DNA, 22 bp, unidentified
  source 1..22: mol_type = other DNA
ttaatggctgcagttgacgaat

SEQ ID NO: 119 - DNA, 20 bp, unidentified
  source 1..20: mol_type = other DNA
cgtcttcgactggcatcatg

SEQ ID NO: 120 - DNA, 30 bp, unidentified
  source 1..30: mol_type = other DNA
cccaactccagaacgtgatactgacaaacc

SEQ ID NO: 121 - DNA, 20 bp, unidentified
  source 1..20: mol_type = other DNA
cgatatgttcccggaattcg

SEQ ID NO: 122 - DNA, 20 bp, unidentified
  source 1..20: mol_type = other DNA
gcgcgcttggtcataaagtt

SEQ ID NO: 123 - DNA, 22 bp, unidentified
  source 1..22: mol_type = other DNA
cggcgattcctctgcccagacc

SEQ ID NO: 124 - DNA, 25 bp, unidentified
  source 1..25: mol_type = other DNA
agataattctttgtcaatttgtgmt

SEQ ID NO: 125 - DNA, 24 bp, unidentified
  source 1..24: mol_type = other DNA
tgaaagaaaactttggctacaacc

SEQ ID NO: 126 - DNA, 25 bp, unidentified
  source 1..25: mol_type = other DNA
atcttgtttgctaaagtcgctacgg

SEQ ID NO: 127 - DNA, 19 bp, unidentified
  source 1..19: mol_type = other DNA
aatgtggcgattgaccgag

SEQ ID NO: 128 - DNA, 21 bp, unidentified
  source 1..21: mol_type = other DNA
ttgagggcactggaaattgta

SEQ ID NO: 129 - DNA, 25 bp, unidentified
  source 1..25: mol_type = other DNA
ccctgctatcaaagactccacaatg

(19) United States
(12) Patent Application Publication
(10) Pub. No.: US 2026/0317838 A1
(43) Pub. Date: Oct. 15, 2026

(54) KIT FOR DETECTING GENITAL TRACT MICROORGANISM RAPIDLY, SYNCHRONOUSLY AND QUANTITATIVELY, AND MICROECOLOGICAL EVALUATION METHOD THEREOF

(71) Applicant: Jiangsu BioPerfectus Technologies Co., Ltd., Taizhou City (CN)

(72) Inventors: Yonghong CHEN, Taizhou (CN); Rong ZHANG, Taizhou (CN); Yuwan GUO, Taizhou (CN); Tengteng LV, Taizhou (CN); Qinghong HUANG, Taizhou (CN); Shan WANG, Taizhou (CN); Xiaokang ZHANG, Taizhou (CN); Zihao NIE, Taizhou (CN); Yao WANG, Taizhou (CN); Ziheng MA, Taizhou (CN); Guohui ZHOU, Taizhou (CN); Zhonghua LIU, Taizhou (CN); Guoqiang WANG, Taizhou (CN)

(21) Appl. No.: 19/359,764

(22) Filed: Oct. 16, 2025

(30) Foreign Application Priority Data
Apr. 10, 2025 (CN) 202510442876.X

(51) Int. Cl. C12Q 1/689; C12Q 1/6893; C12Q 1/6895; C12Q 1/70

(52) U.S. Cl. CPC C12Q 1/689; C12Q 1/6893; C12Q 1/6895; C12Q 1/705; C12Q 1/708; C12Q 2600/166

(57) ABSTRACT

The present invention provides a kit for detecting a genital tract microorganism rapidly, synchronously and quantitatively, and a microecological evaluation method thereof. By designing primer-probe combinations and improving amplification reaction conditions, synchronous and quantitative detection of nearly 50 different types and subtypes of microorganisms in the genital tract can be achieved, covering vaginal lactobacillus groups, pathogenic genital tract microorganisms, sexually transmitted microorganisms, and cervical microorganisms. Moreover, 32 kinds of microorganisms can be distinguished, achieving comprehensive coverage of major microorganisms in the genital tract. Meanwhile, a corresponding microecological evaluation method is also designed in conjunction with the kit, and various vaginitis, sexually transmitted infections, and HPV can be rapidly diagnosed, providing a basis for clinical treatment and prognosis of genital tract infections.

### CROSS-REFERENCE TO RELATED APPLICATIONS

The invention claims priorities to Chinese Patent Application No. 202510442876.X, filed on 10 Apr. 2025, the abstract, specification, claims and accompanying drawings of which are incorporated herein by reference in their entirety as part of this application.

### SEQUENCE LISTING

A sequence listing is being submitted with this application in XML format pursuant to WIPO Standard ST.26. The sequence listing is submitted as a file named “ST26-01.xml”, having a file size of 113,972 bytes, and a date of creation of Dec. 22, 2025. This sequence listing is hereby incorporated by reference in its entirety.

### BACKGROUND OF THE INVENTION

### Field of the Invention

The present invention relates to the field of biomedicine, and specifically to a kit for detecting a genital tract microorganism rapidly, synchronously and quantitatively, and a microecological evaluation method thereof.

### Description of the Related Art

Genital tract infections are common gynecological diseases in women and a global social and public health issue. Common genital tract infectious diseases mainly include vaginitis (bacterial vaginitis, trichomonal vaginitis, candidal vaginitis, and aerobic vaginitis), sexually transmitted infections, and cervical diseases. The main cause of genital tract infections is the invasion of the genital tract by various microorganisms such as bacteria, viruses, mycoplasmas, molds, and trichomonads. In the normal vaginal flora, the reduction or absence of lactobacilli (Lactobacillus crispatus, Lactobacillus gasseri, Lactobacillus jensenii, and Lactobacillus iners) as dominant bacteria weakens the inhibitory effect on other microorganisms. This leads to the massive proliferation of pathogenic microorganisms, thereby causing vaginitis. Bacterial vaginitis, aerobic vaginitis, trichomonal vaginitis, and candidal vaginitis account for more than 70%-90% of all vaginitis cases, among which bacterial vaginitis accounts for about 36%-60% of vaginitis patients, aerobic vaginitis accounts for about 9.4%-23.7% of vaginitis patients, trichomonal vaginitis accounts for about 0.3%-20% of vaginitis patients, and candidal vaginitis accounts for about 15%-40% of vaginitis patients.

The main microorganisms causing bacterial vaginitis include Gardnerella vaginalis, Atopobium vaginae, Prevotella, Mobiluncus, Bacteroides, Megasphaera, bacterial vaginitis-associated bacteria 2 (BVAB2), Ureaplasma urealyticum, Mycoplasma hominis, and the like. The microorganisms causing aerobic vaginitis mainly include Group A Streptococcus, Group B Streptococcus, Escherichia coli, Staphylococcus aureus, Enterococcus faecalis, Streptococcus anginosus, and Klebsiella pneumoniae. The microorganism responsible for trichomonal vaginitis infection is Trichomonas vaginalis. Vulvovaginal candidiasis, also known as vulvovaginal moniliasis, is a genital tract disease caused by Candida infection, whose pathogenic bacteria are mainly Candida albicans, while other non-Candida albicans such as Candida glabrata, Candida tropicalis, and Candida krusei account for a minority. Sexually transmitted infections are a class of widely prevalent diseases, among which gonorrhea is one of the most common types, with Neisseria gonorrhoeae as the main microorganism. In non-gonococcal urethritis, the common microorganism is Chlamydia trachomatis, and Mycoplasma genitalium can also cause non-gonococcal urethritis. Treponema pallidum is the microorganism that causes syphilis, and more than 95% of syphilis infections are transmitted through sexual contact. Herpes simplex virus type 1 and type 2 can cause blister site infections in the genital and perianal areas, leading to sexually transmitted infections. There are more than 200 subtypes of human papillomavirus (HPV) that can infect humans, among which over 40 subtypes can cause human diseases. HPV is mainly transmitted through contact. After persistent infection with HPV, it can cause proliferative lesions in the human genital tract mucosa and skin, which clinically manifest as different types of epithelial warts, as well as malignant tumors such as cervical cancer, anal cancer, and vaginal cancer. Based on the degree of harm of HPV, the World Health Organization (WHO) classifies HPV into 14 high-risk types: HPV16, 18, 31, 33, 35, 39, 45, 51, 52, 56, 58, 59, 66, and 68; and 4 medium-high-risk types: HPV26, 53, 73, and 82; and the rest are low-risk types. Persistent infection with high-risk HPV is the main cause of cervical cancer and its precancerous lesions.

The above microorganisms may cause infection alone, and mixed infection with multiple microorganisms may also occur. Mixed genital tract microbial infections are often accompanied by a complex genital tract environment. Compared with single microbial infection, the diagnosis and treatment of mixed infections are more difficult.

In clinical practice, vaginal microecological detection mainly includes morphological detection and functional detection. Traditional morphological detection, functional detection, culture methods, and serological detection methods are low-cost and easy to operate. However, they rely primarily on morphological observation and determination under a microscope. Not only do these methods require a large amount of labor, but they also have relatively high requirements for the capabilities and experience of laboratory technicians. Additionally, they cannot specifically identify microbial types or perform quantification, nor can they comprehensively and rapidly diagnose genital tract infections caused by multiple microorganisms. Given that detection for mixed genital tract microbial infections takes a relatively long time, there is a clinical need for a method that can rapidly and accurately detect the main microorganisms causing genital tract infections, and diagnose the status of the genital tract microecology based on the detection results.

Real-time fluorescence quantitative polymerase chain reaction (PCR) technology has advantages such as good sensitivity and specificity, and it is a commonly used microbial detection means in clinical practice at present. However, currently available reagents on the market for detecting genital tract infection-causing microorganisms based on molecular diagnostic technology are time-consuming in detection. Moreover, most of these reagents qualitatively detect a single microorganism or several microorganisms that cause a single type of vaginitis; they cannot simultaneously cover the main microorganisms responsible for multiple types of genital tract infections. Furthermore, there are currently no products that have the functions of rapid quantitative detection and microecological evaluation. For example, the primer-probe combination provided in CN117925870A can simultaneously detect 11 types of vaginal microorganisms, including Gardnerella vaginalis, Candida albicans, Candida glabrata, Candida parapsilosis, Candida tropicalis, Candida krusei, Group B Streptococcus, Trichomonas vaginalis, Lactobacillus jensenii, Lactobacillus gasseri, and Lactobacillus crispatus. Detection is performed using a PCR detection device with five fluorescence channels. However, since it can only detect 11 types of microorganisms simultaneously, it is difficult to comprehensively evaluate the status of the genital tract microecology.

Therefore, there is an urgent need to develop a method that can synchronously and quantitatively detect almost all microorganisms with comprehensive coverage in the genital tract within 30 min, and thus can assist in diagnosing multiple types of genital tract infectious diseases and evaluating the genital tract microecology, which holds great significance for realizing multiplex joint detection of genital tract microorganisms and conducting research on sexually transmitted infections.

### BRIEF SUMMARY OF THE INVENTION

In view of the problems existing in the prior art, the present invention provides a kit for detecting a genital tract microorganism rapidly, synchronously and quantitatively, and a microecological evaluation method thereof. Specifically, based on the multiplex fluorescence PCR method, synchronous and quantitative detection of nearly 50 different types and subtypes of microorganisms in the genital tract is performed, covering vaginal lactobacillus groups, pathogenic genital tract microorganisms, sexually transmitted microorganisms, and cervical microorganisms. Moreover, 32 kinds of microorganisms can be distinguished, achieving comprehensive coverage of major microorganisms in the genital tract. Meanwhile, a corresponding microecological evaluation method is also designed in conjunction with the kit, and various vaginitis, sexually transmitted infections, and HPV can be rapidly diagnosed, providing a basis for clinical treatment and prognosis of genital tract infections.

In an aspect, the present invention provides a quantitative evaluation system for detecting genital tract microecology rapidly and synchronously. This system is used to analyze the quantitative detection results of microorganisms in samples, and evaluate the genital tract microecology, realizing the auxiliary diagnosis of various genital tract infectious diseases.

Further, according to the system, quantitative detection of genital tract microorganisms is performed based on standard curves established after multiplex PCR amplification using a combination of multiple primers and probes, and accurate quantification of all target microorganisms is performed through calibration by a high-level metrological traceability system.

Further, according to the system, quantitative detection and analysis are performed on nearly 50 different types and subtypes of genital tract microorganisms, and a supporting evaluation method is adopted to evaluate and analyze the genital tract microecology. This includes determining the status of the genital tract flora and enabling rapid diagnosis of genital tract diseases, where the genital tract diseases include bacterial vaginitis, aerobic vaginitis, vulvovaginal candidiasis, trichomoniasis vaginalis, sexually transmitted infections, and HPV.

Further, the evaluation method is a set of microecological evaluation methods most suitable for the present invention, developed through extensive data analysis after quantitative analysis is performed on 50 different types and subtypes of microorganisms in the present invention. This method does not need to rely on microscopes and inspectors' experience to analyze genital tract diseases like traditional technologies. It can achieve efficient, convenient and accurate microecological evaluation (to determine whether the result of vaginitis is positive or negative) only by using the quantification proportion of a limited number of lactobacillus groups and limited fixed vaginitis-associated microorganisms in samples, which provides a new direction for the diagnosis of genital tract microecology.

According to the quantitative evaluation system for microecology provided by the present invention, rapid diagnosis of bacterial vaginitis and aerobic vaginitis can be achieved by calculating the quantification proportion of lactobacillus groups and vaginitis-associated pathogenic microorganisms in samples. Meanwhile, according to the system, quantitative detection and result interpretation are performed on a variety of microorganisms in samples, including Candida, Trichomonas vaginalis, sexually transmitted infection-associated microorganisms, and human papillomavirus. This achieves rapid diagnosis of vulvovaginal candidiasis, trichomoniasis vaginalis, sexually transmitted infections, and HPV infections. In the present invention, standards are used to compare the quantitative evaluation system for genital tract microecology with the digital PCR method, and the quantitative accuracy of the quantitative evaluation system for genital tract microecology is confirmed. In the present invention, clinical samples are used to compare the quantitative evaluation system for genital tract microecology with the gold standard method and the targeted Next-Generation Sequencing (tNGS) method, and the application accuracy of the quantitative evaluation system for genital tract microecology is confirmed.

In another aspect, the present invention provides a kit for detecting a genital tract microorganism rapidly and synchronously, including any one or more of primer-probe combinations as shown in the table below:

TABLE 1
Primer-probe combinations for 32 kinds of genital tract microorganisms

| Serial | Detected | | Downstream | |
|---|---|---|---|---|
| number | microorganism | Upstream primer | primer | Probe |
| 1 | Lactobacillus | SEQ ID NO. 1 | SEQ ID NO. 2 | SEQ ID NO. 3 |
| | crispatus | | | |
| 2 | Lactobacillus | SEQ ID NO. 4 | SEQ ID NO. 5 | SEQ ID NO. 6 |
| | jensenii | | | |
| 3 | Lactobacillus | SEQ ID NO. 7 | SEQ ID NO. 8 | SEQ ID NO. 9 |
| | gasseri | | | |
| 4 | Lactobacillus iners | SEQ ID NO. 10 | SEQ ID NO. 11 | SEQ ID NO. 12 |
| 5 | Atopobium vaginae | SEQ ID NO. 13 | SEQ ID NO. 14 | SEQ ID NO. 15 |
| 6 | Prevotella | SEQ ID NO. 16 | SEQ ID NO. 17 | SEQ ID NO. 18 |
| 7 | Mycoplasma | SEQ ID NO. 19 | SEQ ID NO. 20 | SEQ ID NO. 21 |
| | genitalium | | | |
| 8 | Mycoplasma | SEQ ID NO. 22 | SEQ ID NO. 23 | SEQ ID NO. 24 |
| | hominis | | | |
| 9 | Gardnerella | SEQ ID NO. 25 | SEQ ID NO. 26 | SEQ ID NO. 27 |
| | vaginalis | | | |
| 10 | Bacteroides | SEQ ID NO. 28 | SEQ ID NO. 29 | SEQ ID NO. 30 |
| 11 | Mobiluncus | SEQ ID NO. 31 | SEQ ID NO. 32 | SEQ ID NO. 33 |
| 12 | Herpes simplex | SEQ ID NO. 34 | SEQ ID NO. 35 | SEQ ID NO. 36 |
| | virus type 2 | | | |
| 13 | Candida albicans | SEQ ID NO. 37 | SEQ ID NO. 38 | SEQ ID NO. 39 |
| 14 | Candida spp. | SEQ ID NO. 40 | SEQ ID NO. 41 | SEQ ID NO. 42 |
| 15 | Escherichia coli | SEQ ID NO. 43 | SEQ ID NO. 44 | SEQ ID NO. 45 |
| 16 | Staphylococcus | SEQ ID NO. 46 | SEQ ID NO. 47 | SEQ ID NO. 48 |
| | aureus | | | |
| 17 | Neisseria | SEQ ID NO. 49 | SEQ ID NO. 50 | SEQ ID NO. 51 |
| | gonorrhoeae | | | |
| 18 | Chlamydia | SEQ ID NO. 52 | SEQ ID NO. 53 | SEQ ID NO. 54 |
| | trachomatis | | | |
| 19 | Ureaplasma | SEQ ID NO. 55 | SEQ ID NO. 56 | SEQ ID NO. 57 |
| | urealyticum | | | |
| 20 | Trichomonas | SEQ ID NO. 58 | SEQ ID NO. 59 | SEQ ID NO. 60 |
| | vaginalis | | | |
| 21 | HPV type 16 | SEQ ID NO. 61 | SEQ ID NO. 62 | SEQ ID NO. 63 |
| 22 | HPV type 18 | SEQ ID NO. 64 | SEQ ID NO. 65 | SEQ ID NO. 66 |
| 23 | HPV type 31 | SEQ ID NO. 67 | SEQ ID NO. 79 | SEQ ID NO. 90 |
| 24 | Group B | SEQ ID NO. 100 | SEQ ID NO. 101 | SEQ ID NO. 102 |
| | Streptococcus | | | |
| 25 | Megasphaera | SEQ ID NO. 103 | SEQ ID NO. 104 | SEQ ID NO. 105 |
| 26 | BVAB2 | SEQ ID NO. 106 | SEQ ID NO. 107 | SEQ ID NO. 108 |
| 27 | Treponema | SEQ ID NO. 109 | SEQ ID NO. 110 | SEQ ID NO. 111 |
| | pallidum | | | |
| 28 | Herpes simplex | SEQ ID NO. 112 | SEQ ID NO. 113 | SEQ ID NO. 114 |
| | virus type 1 | | | |
| 29 | Streptococcus | SEQ ID NO. 115 | SEQ ID NO. 116 | SEQ ID NO. 117 |
| | anginosus | | | |
| 30 | Enterococcus | SEQ ID NO. 118 | SEQ ID NO. 119 | SEQ ID NO. 120 |
| | faecalis | | | |
| 31 | Klebsiella | SEQ ID NO. 121 | SEQ ID NO. 122 | SEQ ID NO. 123 |
| | pneumoniae | | | |
| 32 | Group A | SEQ ID NO. 124 | SEQ ID NO. 125 | SEQ ID NO. 126 |
| | Streptococcus | | | |

In the present invention, the approach of simultaneously monitoring the dominant vaginal flora and the main microorganisms causing genital tract infections is adopted, which assists in diagnosing the status of a patient's genital tract microecology and provides a basis for clinical treatment and prognosis of genital tract infections. It covers the following:
1. 4 dominant bacterial species that are highly prevalent in the normal vaginal flora (Lactobacillus crispatus, Lactobacillus gasseri, Lactobacillus jensenii, and Lactobacillus iners);
2. 9 microorganisms associated with bacterial vaginitis (Gardnerella vaginalis, Prevotella, Mobiluncus, Bacteroides, Atopobium vaginae, Megasphaera, bacterial vaginitis-associated bacterium 2 (BVAB2), Ureaplasma urealyticum, and Mycoplasma hominis). These can cover over 85% of clinical bacterial vaginosis cases, among which Gardnerella vaginalis, Atopobium vaginae, and Prevotella are the most frequently detected bacterial species in clinical practice;
3. 7 microorganisms associated with aerobic vaginitis: Group A Streptococcus, Group B Streptococcus, Escherichia coli, Staphylococcus aureus, Enterococcus faecalis, Streptococcus anginosus, and Klebsiella pneumoniae. These can cover over 90% of clinical cases of aerobic vaginal disease, among which Group B Streptococcus, Escherichia coli, Staphylococcus aureus, and Enterococcus faecalis are the most frequently detected bacterial species in clinical practice;
4. Detection of Trichomonas vaginalis, which can diagnose whether a trichomonal vaginitis infection is present;
5. 4 microorganisms associated with candidal vaginitis (Candida albicans, Candida glabrata, Candida tropicalis, and Candida krusei, where only Candida albicans can be subtyped). These cover over 90% of clinical cases of candidal vaginitis, among which 65%-70% of the microorganisms are Candida albicans, and 10%-20% of the microorganisms are Candida glabrata, Candida tropicalis, and Candida krusei;
6. 6 sexually transmitted pathogenic microorganisms (Treponema pallidum, Neisseria gonorrhoeae, Chlamydia trachomatis, herpes simplex virus type 1, herpes simplex virus type 2, and Mycoplasma genitalium) and 18 types of human papillomavirus (HPV) that cause cervical cancer or genital tract lesions (HPV16, 18, 31, 33, 35, 39, 45, 51, 52, 56, 58, 59, 66, 68, 73, 53, 82, and 26, where only HPV16 and HPV18 can be specifically subtyped). These can cover the most common microorganisms associated with sexually transmitted infections.

In the present invention, specific amplification primers and Taqman probes are designed respectively according to the human genome internal standard fragments and the conserved regions of the respective target genes of 32 kinds of genital tract microorganisms. Whether the corresponding genital tract microorganisms are detected is determined by the positive or negative status of the respective target genes of the microorganisms.

Further, the primer-probe combinations further include a primer-probe combination of an internal standard.

In some embodiments, the internal standard is an RNase P gene, with an upstream primer sequence as shown in SEQ ID NO.127, a downstream primer sequence as shown in SEQ ID NO.128, and a probe sequence as shown in SEQ ID NO.129.

In the present invention, by adding a human genome internal standard, monitoring of processes of sample collection, preservation and transportation, nucleic acid extraction, and PCR amplification can be achieved, thereby avoiding false negative results.

Further, the primer-probe combinations include eight groups of primer-probe combinations as shown in the table below, where each group is capable of achieving synchronous detection of genital tract microorganisms within the group:

TABLE 2
Grouping method of eight groups of primer-probe combinations

| | Detected | | Downstream | |
|---|---|---|---|---|
| Group | microorganism | Upstream primer | primer | Probe |
| First | Lactobacillus | SEQ ID NO. 1 | SEQ ID NO. 2 | SEQ ID NO. 3 |
| group | crispatus | | | |
| | Lactobacillus | SEQ ID NO. 4 | SEQ ID NO. 5 | SEQ ID NO. 6 |
| | jensenii | | | |
| | Lactobacillus | SEQ ID NO. 7 | SEQ ID NO. 8 | SEQ ID NO. 9 |
| | gasseri | | | |
| | Lactobacillus iners | SEQ ID NO. 10 | SEQ ID NO. 11 | SEQ ID NO. 12 |
| Second | Atopobium vaginae | SEQ ID NO. 13 | SEQ ID NO. 14 | SEQ ID NO. 15 |
| group | Prevotella | SEQ ID NO. 16 | SEQ ID NO. 17 | SEQ ID NO. 18 |
| | Mycoplasma | SEQ ID NO. 19 | SEQ ID NO. 20 | SEQ ID NO. 21 |
| | genitalium | | | |
| | Mycoplasma | SEQ ID NO. 22 | SEQ ID NO. 23 | SEQ ID NO. 24 |
| | hominis | | | |
| Third | Gardnerella | SEQ ID NO. 25 | SEQ ID NO. 26 | SEQ ID NO. 27 |
| group | vaginalis | | | |
| | Bacteroides | SEQ ID NO. 28 | SEQ ID NO. 29 | SEQ ID NO. 30 |
| | Mobiluncus | SEQ ID NO. 31 | SEQ ID NO. 32 | SEQ ID NO. 33 |
| | Herpes simplex | SEQ ID NO. 34 | SEQ ID NO. 35 | SEQ ID NO. 36 |
| | virus type 2 | | | |
| Fourth | Candida albicans | SEQ ID NO. 37 | SEQ ID NO. 38 | SEQ ID NO. 39 |
| group | Candida spp. | SEQ ID NO. 40 | SEQ ID NO. 41 | SEQ ID NO. 42 |
| | Escherichia coli | SEQ ID NO. 43 | SEQ ID NO. 44 | SEQ ID NO. 45 |
| | Staphylococcus | SEQ ID NO. 46 | SEQ ID NO. 47 | SEQ ID NO. 48 |
| | aureus | | | |
| Fifth | Neisseria | SEQ ID NO. 49 | SEQ ID NO. 50 | SEQ ID NO. 51 |
| group | gonorrhoeae | | | |
| | Chlamydia | SEQ ID NO. 52 | SEQ ID NO. 53 | SEQ ID NO. 54 |
| | trachomatis | | | |
| | Ureaplasma | SEQ ID NO. 55 | SEQ ID NO. 56 | SEQ ID NO. 57 |
| | urealyticum | | | |
| | Trichomonas | SEQ ID NO. 58 | SEQ ID NO. 59 | SEQ ID NO. 60 |
| | vaginalis | | | |
| Sixth | HPV type 16 | SEQ ID NO. 61 | SEQ ID NO. 62 | SEQ ID NO. 63 |
| group | HPV type 18 | SEQ ID NO. 64 | SEQ ID NO. 65 | SEQ ID NO. 66 |
| | HPV type 31 | SEQ ID NO. 67 | SEQ ID NO. 79 | SEQ ID NO. 90 |
| | Group B | SEQ ID NO. 100 | SEQ ID NO. 101 | SEQ ID NO. 102 |
| | Streptococcus | | | |
| Seventh | Megasphaera | SEQ ID NO. 103 | SEQ ID NO. 104 | SEQ ID NO. 105 |
| group | BVAB2 | SEQ ID NO. 106 | SEQ ID NO. 107 | SEQ ID NO. 108 |
| | Treponema | SEQ ID NO. 109 | SEQ ID NO. 110 | SEQ ID NO. 111 |
| | pallidum | | | |
| | Herpes simplex | SEQ ID NO. 112 | SEQ ID NO. 113 | SEQ ID NO. 114 |
| | virus type 1 | | | |
| Eighth | Streptococcus | SEQ ID NO. 115 | SEQ ID NO. 116 | SEQ ID NO. 117 |
| group | anginosus | | | |
| | Enterococcus | SEQ ID NO. 118 | SEQ ID NO. 119 | SEQ ID NO. 120 |
| | faecalis | | | |
| | Klebsiella | SEQ ID NO. 121 | SEQ ID NO. 122 | SEQ ID NO. 123 |
| | pneumoniae | | | |
| | Group A | SEQ ID NO. 124 | SEQ ID NO. 125 | SEQ ID NO. 126 |
| | Streptococcus | | | |

The 32 groups of primer-probe combinations for microorganisms provided by the present invention can also be pre-grouped and packaged for use. The preferred grouping method is to divide them into eight groups as shown in Table 2. Each group can be placed in one test tube or amplification tube. The kit has 8 amplification tubes, which can enable simultaneous testing of 32 kinds of microorganisms to be detected. The samples are mainly derived from vaginal samples.

Currently, the number of fluorescence channels of real-time fluorescence quantitative PCR instruments available on the market is usually 4-5. Thus, multiplex real-time fluorescence quantitative PCR can detect 3-4 kinds of pathogens and 1 kind of internal standard gene in a system. Therefore, we perform grouped detection of the 32 kinds of microorganisms: 4 types of microorganisms are selected and combined in each group, which are then placed into different sample tubes for simultaneous amplification and quantitative detection. In accordance with the design principles for multiplex primers and probes, the primer pairs and probes for the 4 kinds of microorganisms in the same group are labeled with different fluorescent groups and detected using different channels, with no mutual interference.

The grouping method for the eight groups of microorganisms, as well as the type determination of each microorganism and the design of primer-probe combinations provided by the present invention are all optimal designs finally obtained through extensive screening and optimization experiments. Each group exhibits no cross reaction or mutual interference in the multiplex amplification reaction process, and there is no issue of non-specific amplification, resulting in higher detection accuracy (specificity) and sensitivity.

The present invention has also demonstrated through studies that when Group B Streptococcus in the sixth group and Megasphaera in the seventh group swap positions, or when amplification primers of other groups are interchanged, interference occurs in the amplification process, which affects the detection results of HPV type 16. Therefore, the distribution of primer-probe combinations in each group cannot be arbitrarily changed or combined. Consequently, the amplification primers for the first to eighth groups are all fixed.

Further, the sixth group also includes any one or more of the 15 primer-probe combinations for other high-risk HPV types as shown in Table 3:

TABLE 3
Primer-probe combinations for other high-risk HPV types

| | Detected | | | |
|---|---|---|---|---|
| | micro- | Upstream | Downstream | |
| Group | organism | primer | primer | Probe |
| a1 | HPV type 35 | SEQ ID NO. 68 | SEQ ID NO. 80 | SEQ ID NO. 90 |
| a 2 | HPV type 53 | SEQ ID NO. 69 | SEQ ID NO. 81 | SEQ ID NO. 91 |
| a 3 | HPV types | SEQ ID NO. 70 | SEQ ID NO. 81 | SEQ ID NO. 91 |
| | 56 and 66 | | | |
| a 4 | HPV types | SEQ ID NO. 71 | SEQ ID NO. 82 | SEQ ID NO. 92 |
| | 39 and 68 | | | |
| a 5 | HPV types | SEQ ID NO. 72 | SEQ ID NO. 83 | SEQ ID NO. 93 |
| | 33 and 58 | | | |
| a 6 | HPV types | SEQ ID NO. 73 | SEQ ID NO. 84 | SEQ ID NO. 94 |
| | 51 and 82 | | | |
| a 7 | HPV type 26 | SEQ ID NO. 74 | SEQ ID NO. 85 | SEQ ID NO. 95 |
| a 8 | HPV type 73 | SEQ ID NO. 75 | SEQ ID NO. 86 | SEQ ID NO. 96 |
| a 9 | HPV type 45 | SEQ ID NO. 76 | SEQ ID NO. 87 | SEQ ID NO. 97 |
| a 10 | HPV type 52 | SEQ ID NO. 77 | SEQ ID NO. 88 | SEQ ID NO. 98 |
| a 11 | HPV type 59 | SEQ ID NO. 78 | SEQ ID NO. 89 | SEQ ID NO. 99 |

For other high-risk HPV types, they can also be directly added to the sixth group for simultaneous detection, and the accuracy of the detection results can still be ensured. The primers and probes of some different HPV types can be shared. For example, the probes for HPV type 31 and HPV type 35 can be shared; the primers and probes for HPV types 56 and 66, HPV types 39 and 68, HPV types 33 and 58, as well as HPV types 51 and 82 can all be shared.

In some embodiments, the 15 other high-risk HPV types and HPV type 31 in the sixth group are in one channel, and detection is conducted using the same fluorescent group. Once any one of these types is positive, it can be detected and determined as positive.

In some embodiments, each primer-probe combination in the first group to the eighth group contains an internal standard, and the internal standard is an RNase P gene, with an upstream primer sequence as shown in SEQ ID NO.127, a downstream primer sequence as shown in SEQ ID NO.128, and a probe sequence as shown in SEQ ID NO.129.

In some embodiments, the primer-probe combinations and an internal standard probe are respectively labeled at 5′ terminals with different fluorescent groups and at 3′ terminals with quenching groups.

For the specific probe, the fluorescent group labeled at the 5′ terminal is selected from any one or more of FAM, HEX, VIC, JOE, TET, TAMRA, ROX, QUASAR 705, CY3.5, or CY5; and the quenching group is selected from any one or more of BHQ1, BHQ2, BHQ3, TAMRA, DABCYL, and MGB.

In some embodiments, the probe for a microorganism to be tested is labeled with the fluorescent group FAM/VIC/ROX/CY5 at the 5′ terminal, and the internal standard probe is labeled with the fluorescent group QUASAR 705 at the 5′ terminal.

In the kit of the present invention, all eight groups of primer-probe combinations are added with internal reference genes and are subjected to 5-plex amplification assays, which can effectively monitor the nucleic acid extraction process of samples. Since a fifth fluorescent dye needs to be introduced, the emission spectrum of the dye is prone to overlap with existing channels, resulting in interference between channels, which significantly increases the difficulty compared with the prior art.

In the development process of the kit of the present invention, the inventors solve the problem of typing pathogens with high gene sequence homology through extensive work. For example, both Bacteroides and Prevotella belong to the phylum Bacteroidetes, and their target gene sequences have a homology of about 90%. The inventors design at least 10 groups of primers and probes for each, and only then screen out combinations without cross reaction for use in the kit of the present invention. For the problem of fluorescent labeling interference between 5 channels, suitable labeling combinations are determined through research on probe labeling of different microorganisms. For the problem of non-specific amplification caused by a significant increase in primer dimers, the most suitable groupings and primer-probe combinations are obtained by designing multiple groups of primers, optimizing the concentrations of primers and other reaction system components, or optimizing reaction conditions, and performing extensive screening. For the reduction in reagent stability caused by multiple components, solutions are found by means of optimizing reaction system components and production processes, and the like (such as screening suitable amplification systems).

Further, the kit further includes an amplification reaction solution, where the amplification reaction solution includes a Taq DNA polymerase and a UDG enzyme.

The present invention introduces a UDG enzyme anti-contamination system into the amplification system, which can effectively prevent the occurrence of false positives.

Further, the amplification reaction solution further includes a PCR buffer solution, MgCl₂, dNTPs, and RNase-free water.

In the present invention, a targeted mutation-modified specific Taq DNA polymerase is adopted (Thermo Scientific™ DreamTaq™ DNA polymerase is an enhanced version of Taq DNA polymerase), which helps improve the accuracy and sensitivity of detection. The reason may be that the targeted mutation-modified specific Taq DNA polymerase has more stringent matching initiation than conventional Taq enzymes. At the same time, the purity and concentration of the enzyme antibody are improved, which can effectively block Taq enzyme activity. The enzyme antibody also uses monoclonal antibodies of the active center region. These antibodies exhibit more stable binding, which can effectively prevent non-specific amplification and avoid mutual interference between different primers and probes.

In some embodiments, contents of the DNA polymerase and UDG enzyme are both 3-10 U/Test.

In some embodiments, the PCR buffer solution is a 2×Buffer solution.

In some embodiments, a concentration of the MgCl₂ is 0-10 mM; and a concentration of the dNTPs is 0.5-1 mM, with dATP:dTTP:dCTP:dGTP:dUTP=1:1:1:1:1.

The RNase-free water is used to make up the volume of the reaction solution, and its amount varies according to the concentrations and added amounts of MgCl₂, dNTPs, etc.

In some embodiments, the kit also includes a standard for quantitative detection. The quantitative standard is a WHO standard, a national quantitative standard, a third-party quantitative standard, or a quantified nucleic acid from a target microbial sample, a pseudovirus nucleic acid, or a plasmid DNA. It is obtained by mixing equal volumes of different microbial standards in different combinations, followed by proportional dilution to multiple gradients.

In some embodiments, a method for achieving quantification includes digital PCR quantification, among at least one of methods for standard quantification.

In some embodiments, the kit further includes a positive control and a negative control, where the positive control is at least one of a culture, a plasmid fragment, or a DNA fragment of the above microorganism; and the negative control is at least one of a negative sample and an internal standard plasmid.

It can be understood that the present invention uses PCR as a nucleic acid amplification technology, but it is not limited to PCR alone. Traditional isothermal amplification can still be used to achieve the amplification of the specific primers or specific primer combinations of the present invention. The amplification is also realized by any other technology that can achieve nucleic acid amplification.

In yet another aspect, the present invention provides a microecological evaluation method for detecting a genital tract microorganism rapidly, synchronously and quantitatively. The method adopts the kit as described above to perform nucleic acid amplification on a microorganism from a genital tract sample and perform nucleic acid detection on an amplification product to obtain types and quantities of various microorganisms in a genital tract, thereby performing microecological evaluation and analysis of the genital tract.

The types of microorganisms refer to any one or more of the above 32 kinds of microorganisms; and the quantities of microorganisms refer to concentrations or contents of microorganisms obtained through detection and analysis.

The microecological evaluation method provided by the present invention can perform quantitative detection and microecological evaluation on nearly 50 different types and subtypes of genital tract microorganisms in the sample based on a standard curve for target microbial DNA-CT drawn based on quantitative standards with multiple gradients, thereby realizing auxiliary diagnosis of various genital tract infectious diseases.

In some embodiments, the microecological evaluation method includes the following steps:
(1) nucleic acid extraction from a sample to be tested;
(2) Formulation of reaction systems: formulating reaction systems by respectively using a first primer-probe combination, a second primer-probe combination, a third primer-probe combination, a fourth primer-probe combination, a fifth primer-probe combination, a sixth primer-probe combination, a seventh primer-probe combination, an eighth primer-probe combination, and an internal standard primer-probe combination with the amplification reaction solution;
(3) PCR amplification procedure with the following steps: 30-50° C. for 1 min-10 min; 94-97° C. for 10 s-10 min; and 94-97° C. for 5 s-15 s, and 55-60° C. for 10 s-60 s*, with a total of 40-45 cycles (fluorescence collection at *);
(4) result analysis: if a Ct value of a detection channel FAM/VIC/ROX/CY5 is ≤38.0, a Ct value of a QUASAR 705 channel is ≤37.0, and an amplification curve is S-shaped with an obvious exponential growth phase, determining a corresponding microorganism to be positive; and achieving quantitative detection of the sample according to the standard curve for target microbial DNA-CT drawn based on the quantitative standards with multiple gradients; and
(5) microecological evaluation: establishing a microecological evaluation system to analyze quantification results of a lactobacillus group and a genital tract pathogenic microorganism, respectively, and evaluating genital tract microecology of a patient according to a difference in a ratio of the lactobacillus group to the genital tract pathogenic microorganism.

In some embodiments, the quantitative detection in step (4) refers to quantitative detection of the genital tract microorganism by establishing the standard curve, which is corrected by a high-level metrological traceability system, so that all target microorganisms can be accurately quantified.

Further, evaluation and analysis are performed by adopting the following evaluation method, where the evaluation method includes: any one or more of evaluation of genital tract microecology, evaluation of bacterial vaginitis (BV), evaluation of aerobic vaginitis (AV), evaluation of vulvovaginal candidiasis (VVC), evaluation of trichomoniasis vaginalis (TV), evaluation of sexually transmitted infection (STI), and evaluation of HPV.

Further, the evaluation of genital tract microecology includes: assessing vaginal microecology according to the quantification results of 4 limited vaginal lactobacillus groups: Lactobacillus crispatus, Lactobacillus jensenii, Lactobacillus gasseri, and Lactobacillus iners, where when Lactobacillus crispatus accounts for a relatively high proportion, a result is “normal flora, CSTI”; when Lactobacillus jensenii accounts for a relatively high proportion, a result is “normal flora, CSTV”; when Lactobacillus gasseri accounts for a relatively high proportion, a result is “normal flora, CSTII”; when Lactobacillus iners accounts for a relatively high proportion, a result is “decreased flora function, CSTIII”; and when a proportion of pathogenic microorganisms of bacterial vaginitis (BV) or aerobic vaginitis (AV) is higher than that of the lactobacillus groups, a result is “flora imbalance.”

Further, the evaluation of bacterial vaginitis (BV) includes: obtaining a P value (BV)=(a sum of concentrations of Lactobacillus crispatus, Lactobacillus jensenii, Lactobacillus gasseri, and Lactobacillus iners)÷(a sum of concentrations of Gardnerella vaginalis, Prevotella, Mobiluncus, Bacteroides, Atopobium vaginae, Megasphaera, BVAB2, Ureaplasma urealyticum, and Mycoplasma hominis). Or obtaining a P value (BV) by using a sum of concentrations of Lactobacillus crispatus, Lactobacillus jensenii, Lactobacillus gasseri, and Lactobacillus iners) to divide a sum of concentrations of Gardnerella vaginalis, Prevotella, Mobiluncus, Bacteroides, Atopobium vaginae, Megasphaera, BVAB2, Ureaplasma urealyticum, and Mycoplasma hominis). Where if the P value (BV)≤1, a result is determined as bacterial vaginitis positive; and if the P value (BV)>1, a result is determined as bacterial vaginitis negative.

Although there are various kinds of vaginal lactobacilli (such as Lactobacillus reuteri, Lactobacillus plantarum, Lactobacillus fermentum, etc.), studies by the research team of the present invention have found that accurate assessment of vaginal microecology (whether vaginitis is positive or negative) can be achieved simply by constructing a system using 4 specific microorganisms: Lactobacillus crispatus, Lactobacillus jensenii, Lactobacillus gasseri, and Lactobacillus iners. Moreover, based on these 4 vaginal lactobacilli, assessment of bacterial vaginitis (BV) and aerobic vaginitis (AV) can be performed by using content relationships or ratio relationships of the vaginal lactobacilli with other pathogenic bacteria. Compared with the traditional microscopic examination method, this method is more convenient, accurate, efficient, and cost-saving.

According to the evaluation method of bacterial vaginitis (BV) provided by the present invention, determination can be made simply through nucleic acid detection and quantification results of 4 vaginal lactobacilli and 9 specific pathogenic microorganisms associated with bacterial vaginitis. Specifically, whether a result is bacterial vaginitis positive is assessed according to whether the proportion of the 9 pathogenic microorganisms is higher than that of the 4 lactobacillus groups. The result is more accurate and reliable compared with the traditional microscopic examination method.

Further, the evaluation of aerobic vaginitis (AV) includes: obtaining a P value (AV) by dividing the sum of concentrations of Lactobacillus crispatus, Lactobacillus jensenii, Lactobacillus gasseri, and Lactobacillus iners by a sum of concentrations of Group A Streptococcus, Group B Streptococcus, Staphylococcus aureus, Escherichia coli, Enterococcus faecalis, Streptococcus anginosus, and Klebsiella pneumoniae,
where if the P value (AV)≤1, a result is determined as aerobic vaginitis positive; and if the P value (AV)>1, a result is determined as aerobic vaginitis negative.

According to the evaluation method of aerobic vaginitis (AV) provided by the present invention, determination can be made simply through nucleic acid detection and quantification results of 4 vaginal lactobacilli and 7 pathogenic microorganisms associated with aerobic vaginitis. Specifically, whether a result is aerobic vaginitis positive is assessed according to whether the proportion of the 7 pathogenic microorganisms is higher than that of the 4 lactobacillus groups. The result is more accurate and reliable compared with the traditional microscopic examination method.

Further, the evaluation of vulvovaginal candidiasis (VVC) includes: if Candida spp, and/or Candida albicans is detected as positive, a result is determined as VVC positive; and if Candida spp, and/or Candida albicans is detected as negative, a result is determined as VVC negative.

Further, the evaluation of trichomoniasis vaginalis (TV) includes: if Trichomonas vaginalis is detected as positive, a result is determined as TV positive; and if Trichomonas vaginalis is detected as negative, a result is determined as TV negative.

Further, the evaluation of sexually transmitted infection (STI) includes: if one or more of Neisseria gonorrhoeae, Treponema pallidum, Chlamydia trachomatis, Mycoplasma genitalium, herpes simplex virus type I, and herpes simplex virus type II are detected as positive, a result is determined as STI positive; and if one or more of Neisseria gonorrhoeae, Treponema pallidum, Chlamydia trachomatis, Mycoplasma genitalium, herpes simplex virus type I, and herpes simplex virus type II are detected as negative, a result is determined as STI negative.

Further, the evaluation of HPV includes: if any one or more of HPV16, HPV18, and other high-risk HPV types are detected as positive, a result is determined as HPV positive; and if any one or more of HPV16, HPV18, and other high-risk HPV types are detected as negative, a result is determined as HPV negative.

In yet another aspect, the present invention provides a quantitative evaluation system for detecting genital tract microecology rapidly and synchronously, where the quantitative evaluation system includes the primer-probe combinations as described above and the kit as described above.

Further, the quantitative evaluation system also includes a microecological evaluation system, where the microecological evaluation system is used to perform microecological evaluation on a detection result of the kit.

Further, the microecological evaluation system includes: evaluation of genital tract microecology, evaluation of bacterial vaginitis (BV), evaluation of aerobic vaginitis (AV), evaluation of vulvovaginal candidiasis (VVC), evaluation of trichomoniasis vaginalis (TV), evaluation of sexually transmitted infection (STI), and evaluation of HPV.

Further, the evaluation of genital tract microecology includes: assessing vaginal microecology according to the quantification results of 4 vaginal lactobacillus groups: Lactobacillus crispatus, Lactobacillus jensenii, Lactobacillus gasseri, and Lactobacillus iners, where when Lactobacillus crispatus accounts for a relatively high proportion, a result is “normal flora, CSTI”; when Lactobacillus jensenii accounts for a relatively high proportion, a result is “normal flora, CSTV”; when Lactobacillus gasseri accounts for a relatively high proportion, a result is “normal flora, CSTII”; when Lactobacillus iners accounts for a relatively high proportion, a result is “decreased flora function, CSTIII”; and when a proportion of pathogenic microorganisms of bacterial vaginitis (BV) or aerobic vaginitis (AV) is higher than that of the lactobacillus groups, a result is “flora imbalance”;

The evaluation of bacterial vaginitis (BV) includes: obtaining a P value (BV) by dividing a sum of concentrations of Lactobacillus crispatus, Lactobacillus jensenii, Lactobacillus gasseri, and Lactobacillus iners by a sum of concentrations of Gardnerella vaginalis, Prevotella, Mobiluncus, Bacteroides, Atopobium vaginae, Megasphaera, BVAB2, Ureaplasma urealyticum, and Mycoplasma hominis, where if the P value (BV)≤1, a result is determined as bacterial vaginitis positive; and if the P value (BV)>1, a result is determined as bacterial vaginitis negative.

The evaluation of aerobic vaginitis (AV) includes: obtaining a P value (AV) by dividing the sum of concentrations of Lactobacillus crispatus, Lactobacillus jensenii, Lactobacillus gasseri, and Lactobacillus iners by a sum of concentrations of Group A Streptococcus, Group B Streptococcus, Staphylococcus aureus, Escherichia coli, Enterococcus faecalis, Streptococcus anginosus, and Klebsiella pneumoniae, where if the P value (AV)≤1, a result is determined as aerobic vaginitis positive; and if the P value (AV)>1, a result is determined as aerobic vaginitis negative.

The evaluation of vulvovaginal candidiasis (VVC) includes: if Candida spp. and/or Candida albicans is detected as positive, a result is determined as VVC positive; and if Candida spp, and/or Candida albicans is detected as negative, a result is determined as VVC negative.

The evaluation of trichomoniasis vaginalis (TV) includes: if Trichomonas vaginalis is detected as positive, a result is determined as TV positive; and if Trichomonas vaginalis is detected as negative, a result is determined as TV negative.

The evaluation of sexually transmitted infection (STI) includes: if one or more of Neisseria gonorrhoeae, Treponema pallidum, Chlamydia trachomatis, Mycoplasma genitalium, herpes simplex virus type I, and herpes simplex virus type II are detected as positive, a result is determined as STI positive; and if one or more of Neisseria gonorrhoeae, Treponema pallidum, Chlamydia trachomatis, Mycoplasma genitalium, herpes simplex virus type I, and herpes simplex virus type II are detected as negative, a result is determined as STI negative.

The evaluation of HPV includes: if any one or more of HPV16, HPV18, and other high-risk HPV types are detected as positive, a result is determined as HPV positive; and if any one or more of HPV16, HPV18, and other high-risk HPV types are detected as negative, a result is determined as HPV negative.

In a complex vaginal environment, there are many types of microorganisms. The evaluation of the microecological environment is closely related to subsequent treatment. Through extensive experiments, the present invention has found that the positive or negative status of vaginal inflammation can be directly determined by using the relationship or concentration ratio between 4 types of lactobacilli (beneficial microorganisms) and other limited pathogenic bacteria. This is consistent with actual traditional microbial culture methods, thus forming a diagnostic system from nucleic acid amplification to the determination of inflammation as negative or positive.

Compared with the prior art, the present invention has the following beneficial effects:
(1) nearly 50 different types and subtypes of genital tract microorganisms can be rapidly detected within 30 min, and 32 of the microorganisms can be distinguished, which fully covers common vaginal lactobacillus group, most pathogenic genital tract microorganisms, sexually transmitted microorganisms, and cervical microorganisms, so that rapid detection of single or mixed genital tract microbial infections can be achieved;
(2) the present invention has the advantages of high sensitivity, strong specificity, a wide linear range, good accuracy, rapidity, convenience, and high throughput;
(3) a human genomic internal standard is set up in each group, which can be used to monitor the processes of sample collection, preservation and transportation, nucleic acid extraction, and PCR amplification can be achieved, thereby avoiding false negative results;
(4) a targeted mutation-modified specific Taq enzyme is adopted, which has the advantages of rapidity and specificity, prevents non-specific amplification, and significantly improves the accuracy and sensitivity of detection. Additionally, UDG enzyme is added to effectively avoid the occurrence of false positive results;
(5) the quantitative method for genital tract microorganisms of the present invention is calibrated by a high-level metrological system, so that accurate quantification of all microorganisms within a detection range can be achieved; and
(6) according to the quantitative evaluation system for genital tract microecology of the present invention, the evaluation of genital tract microecology can be achieved by monitoring the proportion of vaginal lactobacilli, such as the determination of dominant bacterial flora, the analysis of flora diversity, and the like, and rapid diagnosis of various vaginitis, sexually transmitted infections, and HPV infections is performed.

### BRIEF DESCRIPTION OF THE DRAWINGS

FIGS. 1-1 to 1-4 show standard curves of combination 1 in Example 2, where FIG. 1-1 shows a standard curve of Lactobacillus crispatus, FIG. 1-2 shows a standard curve of Lactobacillus jensenii, FIG. 1-3 shows a standard curve of Lactobacillus gasseri, and FIG. 1-4 shows a standard curve of Lactobacillus iners;

FIGS. 2-1 to 2-4 show standard curves of combination 3 in Example 2, where FIG. 2-1 shows a standard curve of Gardnerella vaginalis, FIG. 2-2 shows a standard curve of Bacteroides, FIG. 2-3 shows a standard curve of Mobiluncus, and FIG. 2-4 shows a standard curve of herpes simplex virus type 2;

FIGS. 3-1 to 3-4 show standard curves of combination 6 in Example 2, where FIG. 3-1 shows a standard curve of HPV type 16, FIG. 3-2 shows a standard curve of HPV type 18, FIG. 3-3 shows a standard curve of HPV type 31, and FIG. 3-4 shows a standard curve of Group B Streptococcus (GBS);

FIGS. 4-1 to 4-8 show detection results of simulated samples 1-8 in Example 3, where FIG. 4-1 shows detection results of simulated sample 1, FIG. 4-2 shows detection results of simulated sample 2, FIG. 4-3 shows detection results of simulated sample 3, FIG. 4-4 shows detection results of simulated sample 4, FIG. 4-5 shows detection results of simulated sample 5, FIG. 4-6 shows detection results of simulated sample 6, FIG. 4-7 shows detection results of simulated sample 7, and FIG. 4-8 shows detection results of simulated sample 8;

FIGS. 5-1 to 5-4 show sensitivity detection results of primer-probe combinations of combination 1 in Example 3 for each target, where FIG. 5-1 shows sensitivity detection results of a primer-probe combination of combination 1 for Lactobacillus crispatus, FIG. 5-2 shows sensitivity detection results of a primer-probe combination of combination 1 for Lactobacillus jensenii, FIG. 5-3 shows sensitivity detection results of a primer-probe combination of combination 1 for Lactobacillus gasseri, and FIG. 5-4 shows sensitivity detection results of a primer-probe combination of combination 1 for Lactobacillus iners;

FIGS. 6-1 to 6-4 show sensitivity detection results of primer-probe combinations of combination 2 in Example 3 for each target, where FIG. 6-1 shows sensitivity detection results of a primer-probe combination of combination 2 for Atopobium vaginae, FIG. 6-2 shows sensitivity detection results of a primer-probe combination of combination 2 for Prevotella, FIG. 6-3 shows sensitivity detection results of a primer-probe combination of combination 2 for Mycoplasma genitalium, and FIG. 6-4 shows sensitivity detection results of a primer-probe combination of combination 2 for Mycoplasma hominis;

FIGS. 7-1 to 7-4 show sensitivity detection results of primer-probe combinations of combination 3 in Example 3 for each target, where FIG. 7-1 shows sensitivity detection results of a primer-probe combination of combination 3 for Gardnerella vaginalis, FIG. 7-2 shows sensitivity detection results of a primer-probe combination of combination 3 for Bacteroides, FIG. 7-3 shows sensitivity detection results of a primer-probe combination of combination 3 for Mobiluncus, and FIG. 7-4 shows sensitivity detection results of a primer-probe combination of combination 3 for herpes simplex virus type 2;

FIGS. 8-1 to 8-4 show sensitivity detection results of primer-probe combinations of combination 4 in Example 3 for each target, where FIG. 8-1 shows sensitivity detection results of a primer-probe combination of combination 4 for Candida albicans, FIG. 8-2 shows sensitivity detection results of a primer-probe combination of combination 4 for Candida spp., FIG. 8-3 shows sensitivity detection results of a primer-probe combination of combination 4 for Escherichia coli, and FIG. 8-4 shows sensitivity detection results of a primer-probe combination of combination 4 for Staphylococcus aureus;

FIGS. 9-1 to 9-4 show sensitivity detection results of primer-probe combinations of combination 5 in Example 3 for each target, where FIG. 9-1 shows sensitivity detection results of a primer-probe combination of combination 5 for Neisseria gonorrhoeae, FIG. 9-2 shows sensitivity detection results of a primer-probe combination of combination 5 for Chlamydia trachomatis, FIG. 9-3 shows sensitivity detection results of a primer-probe combination of combination 5 for Ureaplasma urealyticum, and FIG. 9-4 shows sensitivity detection results of a primer-probe combination of combination 5 for Trichomonas vaginalis;

FIGS. 10-1 to 10-4 show sensitivity detection results of primer-probe combinations of combination 6 in Example 3 for each target, where FIG. 10-1 shows sensitivity detection results of a primer-probe combination of combination 6 for HPV type 16, FIG. 10-2 shows sensitivity detection results of a primer-probe combination of combination 6 for HPV type 18, FIG. 10-3 shows sensitivity detection results of a primer-probe combination of combination 6 for HPV type 31, and FIG. 10-4 shows sensitivity detection results of a primer-probe combination of combination 6 for Group B Streptococcus;

FIGS. 11-1 to 11-4 show sensitivity detection results of primer-probe combinations of combination 7 in Example 3 for each target, where FIG. 11-1 shows sensitivity detection results of a primer-probe combination of combination 7 for Treponema pallidum, FIG. 11-2 shows sensitivity detection results of a primer-probe combination of combination 7 for BVAB2, FIG. 11-3 shows sensitivity detection results of a primer-probe combination of combination 7 for Megasphaera, and FIG. 11-4 shows sensitivity detection results of a primer-probe combination of combination 7 for herpes simplex virus type 1;

FIGS. 12-1 to 12-4 show sensitivity detection results of primer-probe combinations of combination 8 in Example 3 for each target, where FIG. 12-1 shows sensitivity detection results of a primer-probe combination of combination 8 for Streptococcus anginosus, FIG. 12-2 shows sensitivity detection results of a primer-probe combination of combination 8 for Enterococcus faecalis, FIG. 12-3 shows sensitivity detection results of a primer-probe combination of combination 8 for Klebsiella pneumoniae, and FIG. 12-4 shows sensitivity detection results of a primer-probe combination of combination 8 for Group A Streptococcus;

FIG. 13 shows specificity detection results of each group of primer-probe combinations in Example 3;

FIGS. 14-1 to 14-4 show precision detection results of each group of primer-probe combinations from combination 1 to combination 4 in Example 3, where FIG. 14-1 shows precision detection results of each group of primer-probe combinations of combination 1, FIG. 14-2 shows precision detection results of each group of primer-probe combinations of combination 2, FIG. 14-3 shows precision detection results of each group of primer-probe combinations of combination 3, and FIG. 14-4 shows precision detection results of each group of primer-probe combinations of combination 4;

FIGS. 15-1 to 15-4 show precision detection results of each group of primer-probe combinations from combination 5 to combination 8 in Example 3, where FIG. 15-1 shows precision detection results of each group of primer-probe combinations of combination 5, FIG. 15-2 shows precision detection results of each group of primer-probe combinations of combination 6, FIG. 15-3 shows precision detection results of each group of primer-probe combinations of combination 7, and FIG. 15-4 shows precision detection results of each group of primer-probe combinations of combination 8; and

FIGS. 16-1 to 16-4 show schematic diagrams of results obtained by detection through a microecological evaluation system in Example 4, where FIG. 16-1 shows a schematic diagram of results obtained by detection of sample 1 through a microecological evaluation system, FIG. 16-2 shows a schematic diagram of results obtained by detection of sample 11 through a microecological evaluation system, FIG. 16-3 shows a schematic diagram of results obtained by detection of sample 18 through a microecological evaluation system, and FIG. 16-4 shows a schematic diagram of results obtained by detection of sample 21 through a microecological evaluation system.

### DETAILED DESCRIPTION OF THE INVENTION

The present invention is described in further detail below with reference to examples. It should be noted that the following examples are intended to facilitate the understanding of the present invention and do not impose any limitation thereon. All reagents used in the examples are known products and are obtained by purchasing commercially available products.

### Example 1. Primer and Probe Sequences and Combinations for Detecting Genital Tract Microorganisms

In the present invention, specific primer and probe sequences are designed by taking the conserved regions of the genomes of various genital tract microorganisms and internal standard genes as targets. The primer and probe sequences for each microorganism are shown in Table 4.

TABLE 4
Primer and probe sequences for genital tract microorganisms

| Serial | Detected | | Downstream | |
|---|---|---|---|---|
| number | microorganism | Upstream primer | primer | Probe |
| 1 | Lactobacillus | SEQ ID NO. 1 | SEQ ID NO. 2 | SEQ ID NO. 3 |
| | crispatus | | | |
| 2 | Lactobacillus | SEQ ID NO. 4 | SEQ ID NO. 5 | SEQ ID NO. 6 |
| | jensenii | | | |
| 3 | Lactobacillus | SEQ ID NO. 7 | SEQ ID NO. 8 | SEQ ID NO. 9 |
| | gasseri | | | |
| 4 | Lactobacillus iners | SEQ ID NO. 10 | SEQ ID NO. 11 | SEQ ID NO. 12 |
| 5 | Atopobium vaginae | SEQ ID NO. 13 | SEQ ID NO. 14 | SEQ ID NO. 15 |
| 6 | Prevotella | SEQ ID NO. 16 | SEQ ID NO. 17 | SEQ ID NO. 18 |
| 7 | Mycoplasma | SEQ ID NO. 19 | SEQ ID NO. 20 | SEQ ID NO. 21 |
| | genitalium | | | |
| 8 | Mycoplasma | SEQ ID NO. 22 | SEQ ID NO. 23 | SEQ ID NO. 24 |
| | hominis | | | |
| 9 | Gardnerella | SEQ ID NO. 25 | SEQ ID NO. 26 | SEQ ID NO. 27 |
| | vaginalis | | | |
| 10 | Bacteroides | SEQ ID NO. 28 | SEQ ID NO. 29 | SEQ ID NO. 30 |
| 11 | Mobiluncus | SEQ ID NO. 31 | SEQ ID NO. 32 | SEQ ID NO. 33 |
| 12 | Herpes simplex | SEQ ID NO. 34 | SEQ ID NO. 35 | SEQ ID NO. 36 |
| | virus type 2 | | | |
| 13 | Candida albicans | SEQ ID NO. 37 | SEQ ID NO. 38 | SEQ ID NO. 39 |
| 14 | Candida spp. | SEQ ID NO. 40 | SEQ ID NO. 41 | SEQ ID NO. 42 |
| 15 | Escherichia coli | SEQ ID NO. 43 | SEQ ID NO. 44 | SEQ ID NO. 45 |
| 16 | Staphylococcus | SEQ ID NO. 46 | SEQ ID NO. 47 | SEQ ID NO. 48 |
| | aureus | | | |
| 17 | Neisseria | SEQ ID NO. 49 | SEQ ID NO. 50 | SEQ ID NO. 51 |
| | gonorrhoeae | | | |
| 18 | Chlamydia | SEQ ID NO. 52 | SEQ ID NO. 53 | SEQ ID NO. 54 |
| | trachomatis | | | |
| 19 | Ureaplasma | SEQ ID NO. 55 | SEQ ID NO. 56 | SEQ ID NO. 57 |
| | urealyticum | | | |
| 20 | Trichomonas | SEQ ID NO. 58 | SEQ ID NO. 59 | SEQ ID NO. 60 |
| | vaginalis | | | |
| 21 | HPV type 16 | SEQ ID NO. 61 | SEQ ID NO. 62 | SEQ ID NO. 63 |
| 22 | HPV type 18 | SEQ ID NO. 64 | SEQ ID NO. 65 | SEQ ID NO. 66 |
| 23 | HPV type 31 | SEQ ID NO. 67 | SEQ ID NO. 79 | SEQ ID NO. 90 |
| 24 | HPV type 35 | SEQ ID NO. 68 | SEQ ID NO. 80 | SEQ ID NO. 90 |
| 25 | HPV type 53 | SEQ ID NO. 69 | SEQ ID NO. 81 | SEQ ID NO. 91 |
| 26 | HPV types 56 and | SEQ ID NO. 70 | SEQ ID NO. 81 | SEQ ID NO. 91 |
| | 66 | | | |
| 27 | HPV types 39 and | SEQ ID NO. 71 | SEQ ID NO. 82 | SEQ ID NO. 92 |
| | 68 | | | |
| 28 | HPV types 33 and | SEQ ID NO. 72 | SEQ ID NO. 83 | SEQ ID NO. 93 |
| | 58 | | | |
| 29 | HPV types 51 and | SEQ ID NO. 73 | SEQ ID NO. 84 | SEQ ID NO. 94 |
| | 82 | | | |
| 30 | HPV type 26 | SEQ ID NO. 74 | SEQ ID NO. 85 | SEQ ID NO. 95 |
| 31 | HPV type 73 | SEQ ID NO. 75 | SEQ ID NO. 86 | SEQ ID NO. 96 |
| 32 | HPV type 45 | SEQ ID NO. 76 | SEQ ID NO. 87 | SEQ ID NO. 97 |
| 33 | HPV type 52 | SEQ ID NO. 77 | SEQ ID NO. 88 | SEQ ID NO. 98 |
| 34 | HPV type 59 | SEQ ID NO. 78 | SEQ ID NO. 89 | SEQ ID NO. 99 |
| 35 | Group B | SEQ ID NO. 100 | SEQ ID NO. 101 | SEQ ID NO. 102 |
| | Streptococcus | | | |
| 36 | Megasphaera | SEQ ID NO. 103 | SEQ ID NO. 104 | SEQ ID NO. 105 |
| 37 | BVAB2 | SEQ ID NO. 106 | SEQ ID NO. 107 | SEQ ID NO. 108 |
| 38 | Treponema | SEQ ID NO. 109 | SEQ ID NO. 110 | SEQ ID NO. 111 |
| | pallidum | | | |
| 39 | Herpes simplex | SEQ ID NO. 112 | SEQ ID NO. 113 | SEQ ID NO. 114 |
| | virus type 1 | | | |
| 40 | Streptococcus | SEQ ID NO. 115 | SEQ ID NO. 116 | SEQ ID NO. 117 |
| | anginosus | | | |
| 41 | Enterococcus | SEQ ID NO. 118 | SEQ ID NO. 119 | SEQ ID NO. 120 |
| | faecalis | | | |
| 42 | Klebsiella | SEQ ID NO. 121 | SEQ ID NO. 122 | SEQ ID NO. 123 |
| | pneumoniae | | | |
| 43 | Group A | SEQ ID NO. 124 | SEQ ID NO. 125 | SEQ ID NO. 126 |
| | Streptococcus | | | |

The primers and probes for each of the above microorganisms are obtained through extensive screening. They can specifically detect the type and quantity of each microorganism, with high specificity and high sensitivity.

To simplify the operation, in the present invention, the above genital tract microorganisms and internal standard genes are divided into eight primer-probe combinations. The 5′ terminal of each probe is labeled with a fluorescent group FAM/VIC/ROX/CY5/QUASAR 705, and the 3′ terminal of each probe is labeled with a quenching group BHQ1/BHQ2/BHQ3. The grouping method and the setting method of fluorescent groups are shown in Table 5 below.

TABLE 5
Grouping method and setting method of fluorescent groups

| | Channel | | | | |
|---|---|---|---|---|---|
| Primer- | | | | | QUASAR |
| probe | | | | | 705 |
| combination | FAM channel | VIC channel | ROX channel | CY5 channel | channel |
| Combination | Lactobacillus | Lactobacillus | Lactobacillus | Lactobacillus | Internal |
| 1 | crispatus | jensenii | gasseri | iners | standard |
| Combination | Atopobium | Prevotella | Mycoplasma | Mycoplasma | Internal |
| 2 | vaginae | | genitalium | hominis | standard |
| Combination | Gardnerella | Bacteroides | Mobiluncus | Herpes simplex | Internal |
| 3 | vaginalis | | | virus type 2 | standard |
| Combination | Candida | Candida spp. | Escherichia | Staphylococcus | Internal |
| 4 | albicans | | coli | aureus | standard |
| Combination | Neisseria | Chlamydia | Ureaplasma | Trichomonas | Internal |
| 5 | gonorrhoeae | trachomatis | urealyticum | vaginalis | standard |
| Combination | HPV type 16 | HPV type 18 | Other high- | Group B | Internal |
| 6 | | | risk HPV | Streptococcus | standard |
| | | | types | | |
| Combination | Megasphaera | BVAB2 | Treponema | Herpes simplex | Internal |
| 7 | | | pallidum | virus type 1 | standard |
| Combination | Streptococcus | Enterococcus | Klebsiella | Group A | Internal |
| 8 | anginosus | faecalis | pneumoniae | Streptococcus | standard |

Thus, the design and screening of the above primers and probes not only can meet the requirements for specificity and sensitivity when detecting a single microorganism in a single tube, but also need to ensure no cross amplification in the case of different combinations while still satisfying the requirements for specificity and sensitivity. After multiple different combinations were performed, the combinations shown in Table 5 were finally obtained, so that multiplex amplification can be performed simultaneously in a single tube, realizing the distinction of 4 different microorganisms and meeting the sensitivity requirement. In addition, each group requires the addition of internal standard primers, which means that 5-plex amplification needs to be achieved in an amplification tube. In this way, simultaneous testing can be performed for 32 microorganisms, with each test enabling the identification and quantity testing of 4 microorganisms at one time.

In some embodiments, the ROX channel shown in combination 6 includes 12 groups of primers and probes for HPV types with serial numbers 23-34 in Table 4 or the primers and probes in Table 3. All these 12 groups of probes use ROX fluorescent groups. By using the same fluorescent group to detect 15 other high-risk HPV types, once any one of these types is positive, it can be detected.

This is equivalent to adding 12 additional groups of primers to the existing primer combinations in combination 6 of Table 5, enabling the amplification of 18 different groups of primers. Although the 12 added groups of amplification primers adopt the same luminescent labeling substance and detect the total value, achieving both high specificity and high sensitivity remains highly challenging. Meanwhile, the normal amplification of Group B Streptococcus, HPV type 16, and HPV type 18 cannot be affected, making the design and combination of the primers extremely difficult. After repeated experiments and screening by the research team of the present invention, a specific different microbial combination in combination 6 was finally selected to achieve the above objectives. For example, if Group B Streptococcus is replaced with any other microorganism in combination 6, it is almost impossible to distinguish between different microorganisms. For instance, distinction between HPV type 16 and HPV type 18 cannot be achieved. This may be due to cross amplification, resulting in low specificity, and in some cases, no results can even be detected.

The grouping method for the eight groups of microorganisms, as well as the type determination of each microorganism and the design of primer-probe combinations provided in this example were all optimal designs finally obtained through extensive screening and optimization experiments. Each group exhibited no cross reaction or mutual interference in the multiplex amplification reaction process, and there was no issue of non-specific amplification, resulting in higher detection accuracy and sensitivity.

In the kit provided in this example, all eight groups of primer-probe combinations were added with internal reference genes (RNase P gene, with an upstream primer sequence as shown in SEQ ID NO.127, a downstream primer sequence as shown in SEQ ID NO.128, and a probe sequence as shown in SEQ ID NO.129), and were subjected to 5-plex amplification assays, which can effectively monitor the nucleic acid extraction process of samples. Since a fifth fluorescent dye needs to be introduced, the emission spectrum of the dye is prone to overlap with existing channels, resulting in interference between channels, which significantly increases the difficulty compared with the prior art.

In the development process of the kit provided in this example, the inventors solved the problem of typing pathogens with high gene sequence homology through extensive work. For example, both Bacteroides and Prevotella belong to the phylum Bacteroidetes, and their target gene sequences have a homology of about 90%. The inventors designed at least 10 groups of primers and probes for each, and only then screened out combinations without cross reaction for use in the kit provided in this example. For the problem of fluorescent labeling interference between 5 channels, suitable labeling combinations were determined through research on probe labeling of different microorganisms. For the problem of non-specific amplification caused by a significant increase in primer dimers, the most suitable groupings and primer-probe combinations were obtained by designing multiple groups of primers, optimizing the concentrations of primers and other reaction system components, or optimizing reaction conditions, and performing extensive screening. For the reduction in reagent stability caused by multiple components, solutions were found by means of optimizing reaction system components and production processes, and the like (such as screening suitable amplification systems).

### Example 2. Method for Detecting Genital Tract Microorganisms Synchronously and Quantitatively Provided by the Present Invention

This example performed detection using a five-channel real-time fluorescence quantitative PCR instrument based on the grouping method and the setting method of fluorescent groups of each group's probes as shown in Example 1.

### I. Specific Detection Method

### 1. Formulation of Reaction Systems

The amplification reaction system was formulated in accordance with Table 5: 8 tubes of primer-probe combinations and the amplification reaction solution were respectively taken, added to reagent tubes in proportion, mixed thoroughly to form reaction systems 1 to 8, and subjected to instantaneous centrifugation for later use.

TABLE 5
Reaction system formulation

| | Addition amount |
|---|---|
| Component | (μL)/person |
| Amplification reaction | 12.5 |
| solution | |
| Primer-probe combination | 7.5 |
| (1-8) | |
| Total volume | 20 |

The amplification reaction solution contain 3-10 U/Test (preferably 8 U/Test in this example) of Taq DNA polymerase (purchased from Shanghai Shuoying Biotechnology Co., Ltd., model CSMix-005), 3-10 U/Test (preferably 8 U/Test in this example) of UDG enzyme, 2×Buffer solution, 0-10 mM (preferably 5 mM in this example) of MgCl₂, and 0.5-1 mM (preferably 1 mM in this example) of dNTPs (dATP:dTTP:dCTP:dGTP:dUTP=1:1:1:1:1), which was prepared with RNase-free water.

2. Nucleic Acid Extraction from Samples

In this example, clinically collected cervical/vaginal swab samples were adopted (vaginal swab samples were adopted in this example), and nucleic acid extraction was performed using the nucleic acid extraction and purification kit (magnetic bead method) from Jiangsu Bioperfectus Technologies Co., Ltd. Positive controls, negative controls, and quantitative standards were taken for simultaneous nucleic acid extraction. Another example is the samples collected by the applicant with sanitary pads in Chinese Application (application No. 2025108405593).

### 3. Sample Loading

5 μL each of the extracted positive control, negative control, quantitative standard, and nucleic acid from the test sample were added to the above prepared reagent tubes, respectively. The tube lids were tightly closed, followed by instantaneous low-speed centrifugation.

### 4. Amplification Reaction Procedure

The above reagent tubes were placed into an SLAN-96H fluorescence PCR instrument. The names of samples to be tested, standards, and their corresponding concentrations were set, and the amplification detection was initiated. The conditions for the multiplex fluorescence PCR reaction are shown in Table 6.

TABLE 6
Amplification procedure

| Step | Temperature (°C) | Time | Number of cycles |
|---|---|---|---|
| 1 | 50 °C | 2 min | 1 |
| 2 | 95 °C | 10 s | 1 |
| 3 | 95 °C | 5 s | 45 |
| | 58 °C* | 20 s | |

In step 3, fluorescence detection was performed at 58 °C
Detection channels: FAM, VIC, ROX, CY5, and QUASAR 705.

### II. Result Analysis

Results were automatically interpreted by software. The default baseline started at cycle 6 and ended at cycle 12, with a threshold value of 0.12. “Analyze” was clicked to automatically obtain analysis results. The positive control and negative control shall meet the requirements in the “quality control” section below. Quality control is as follows:
(1) positive control: the Ct values of FAM, VIC, ROX, CY5, and QUASAR 705 channels shall be ≤30.0; and (2) negative control: no Ct values (NoCt) shall be detected in FAM, VIC, ROX, and CY5 channels, and the Ct value of the QUASAR 705 channel shall be ≤30.0.

The above requirements need to be met simultaneously in the same experiment; otherwise, the experiment shall be deemed invalid.

If the Ct value of the detection channel FAM/VIC/ROX/CY5 is ≤38.0, the Ct value of the QUASAR 705 channel is ≤37.0, and an amplification curve is S-shaped with an obvious exponential growth phase, a corresponding microorganism is determined to be positive.

### 1. Quantitative Analysis

### (1) Establishment of Standard Curves

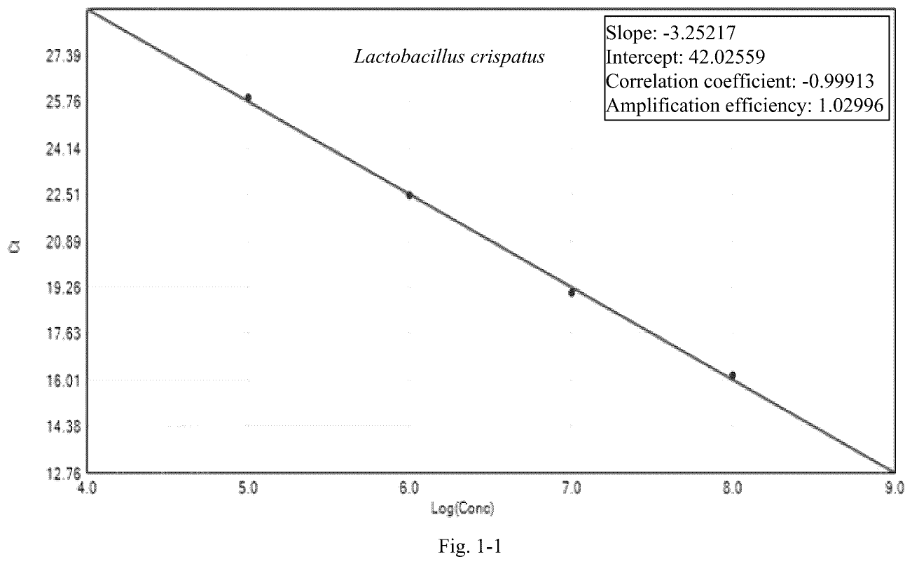

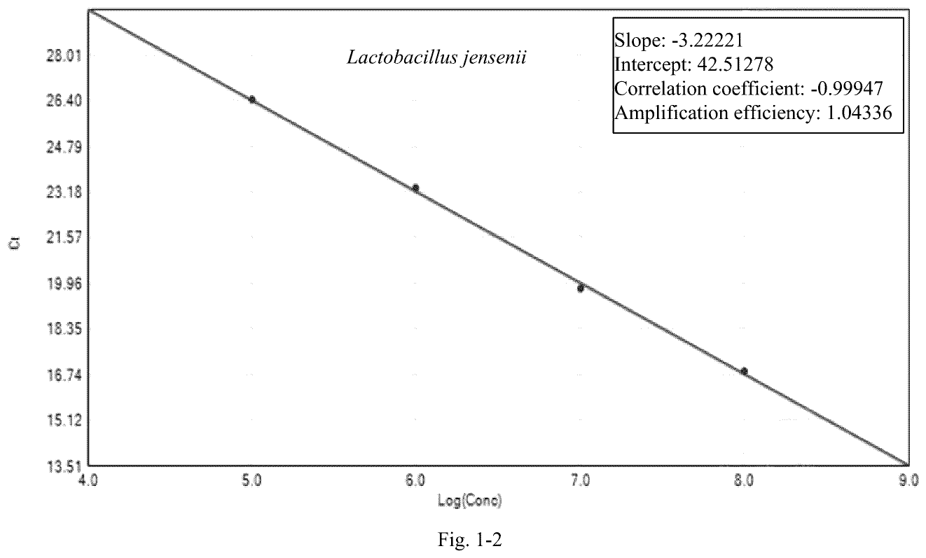

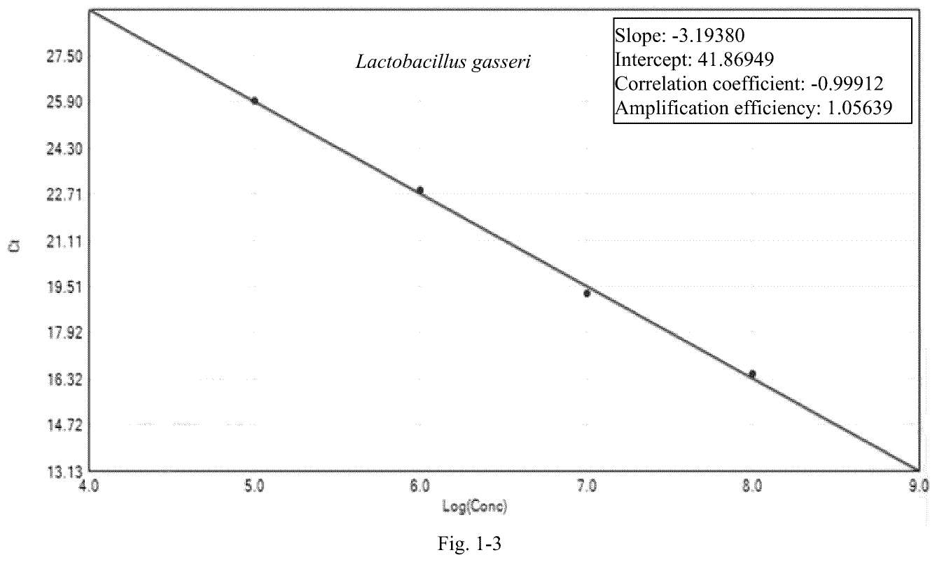

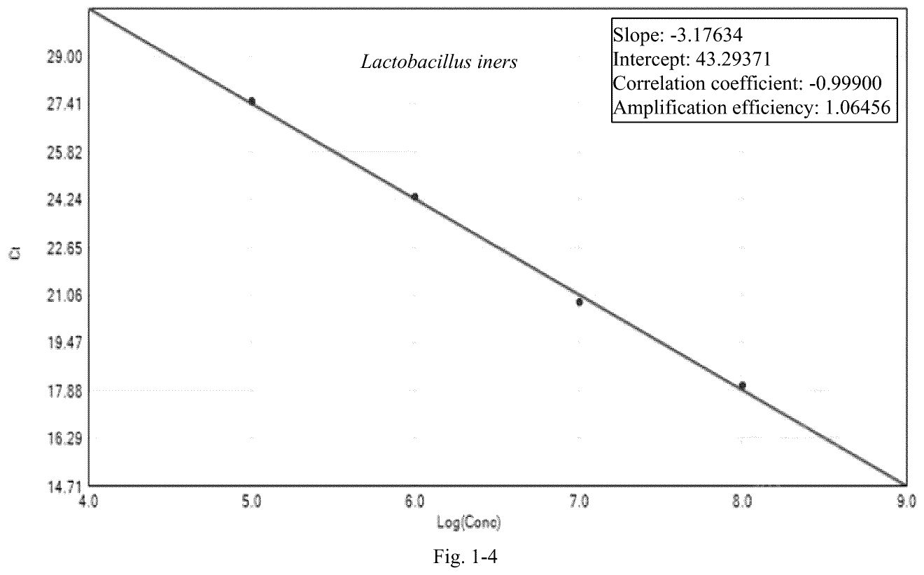

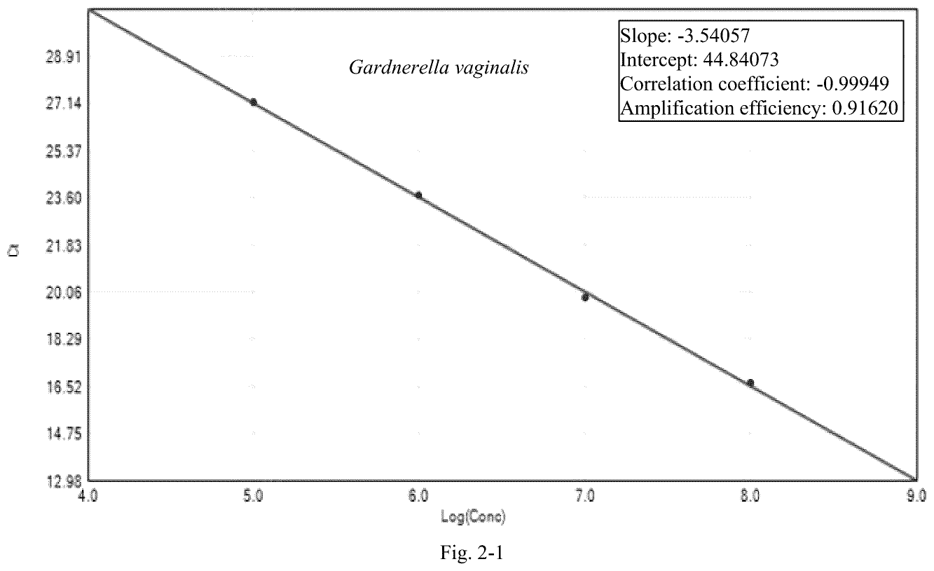

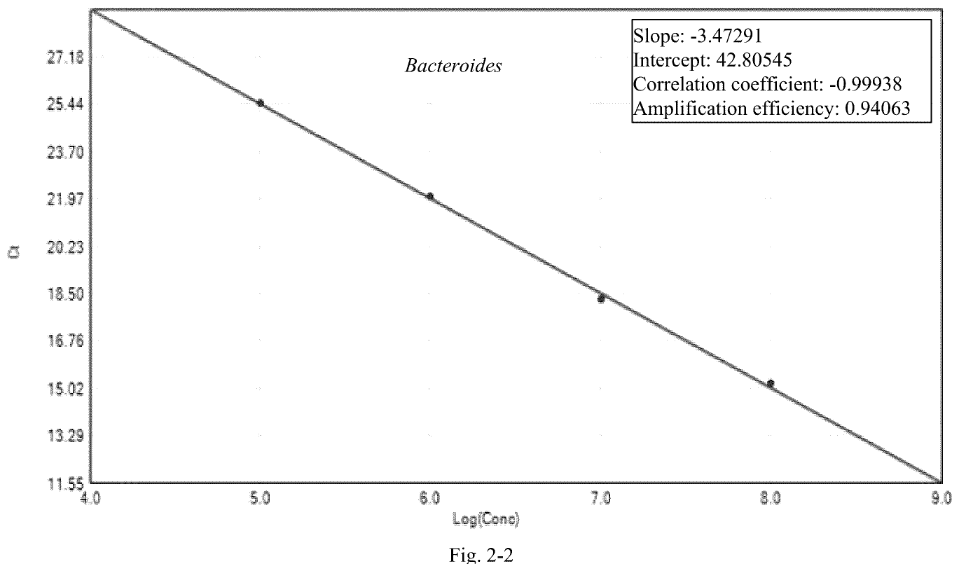

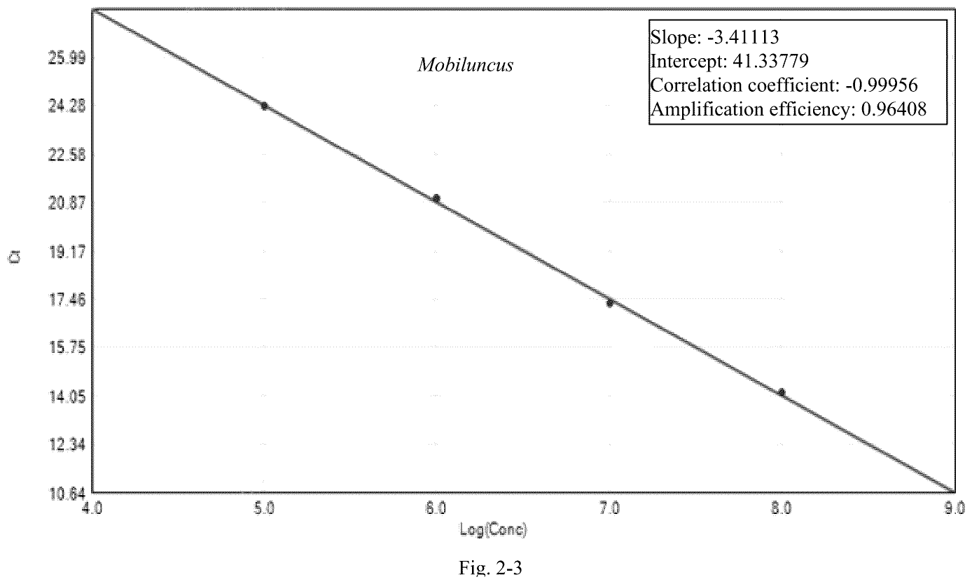

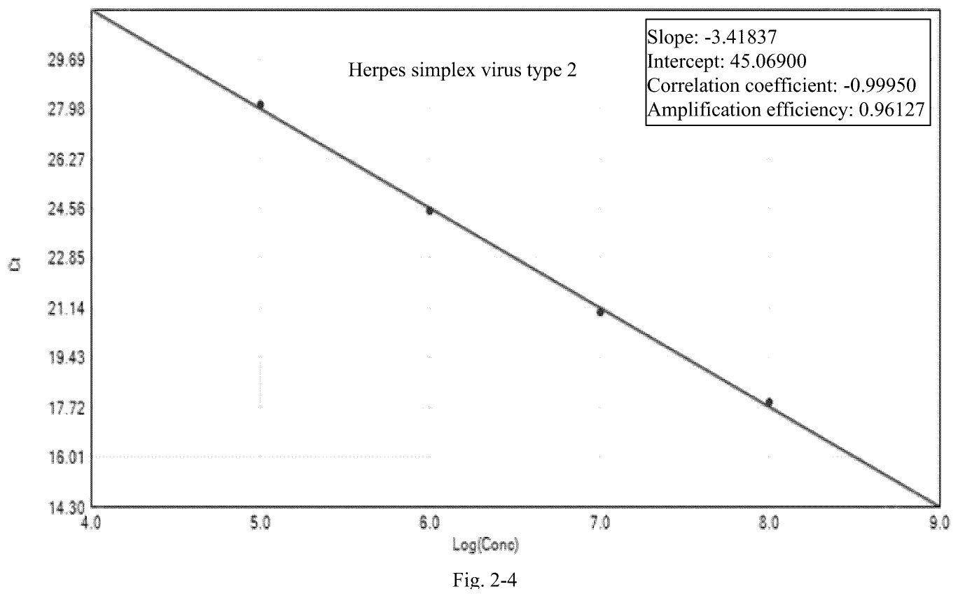

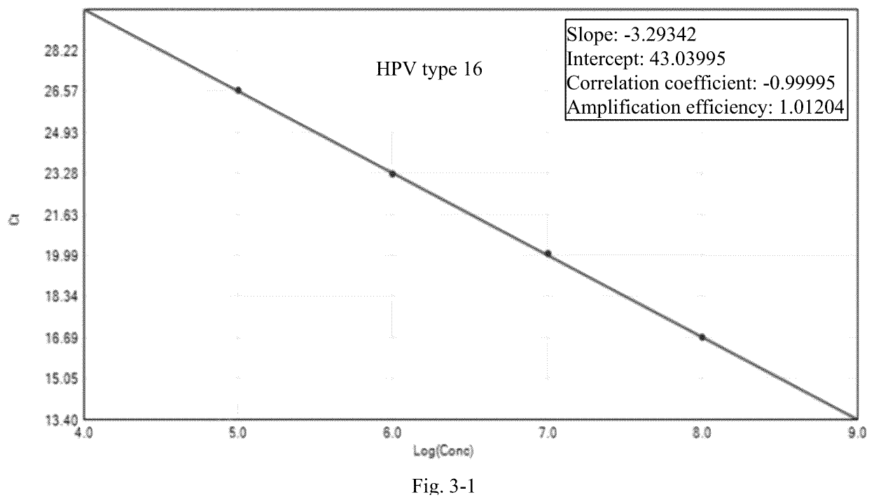

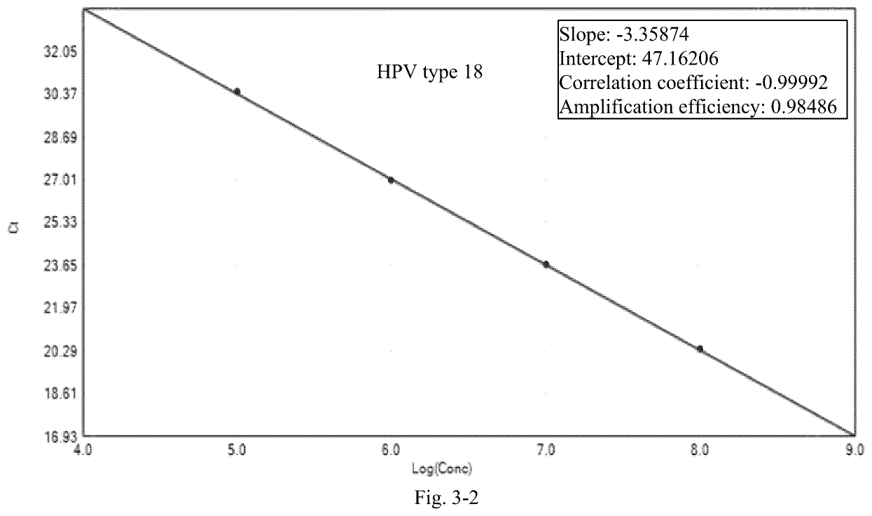

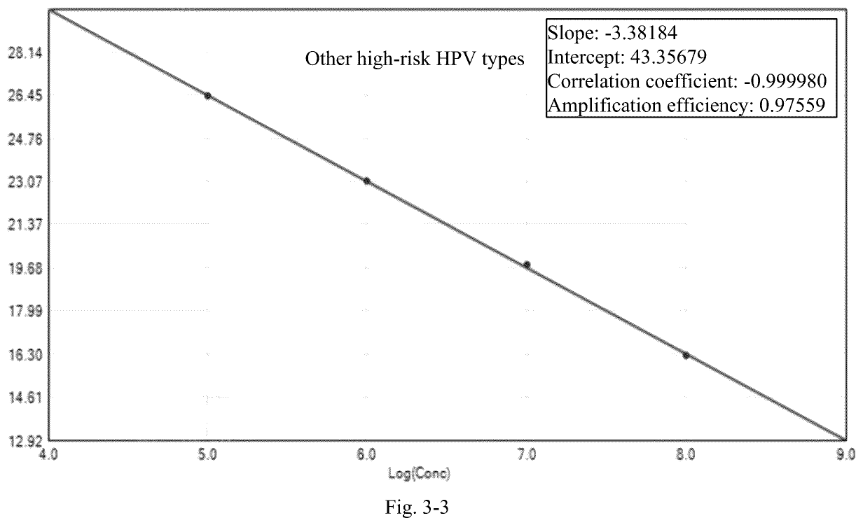

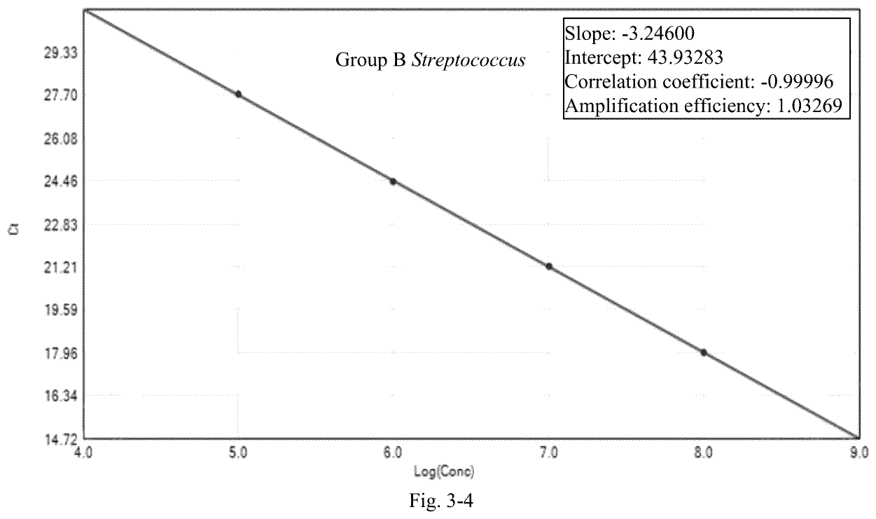

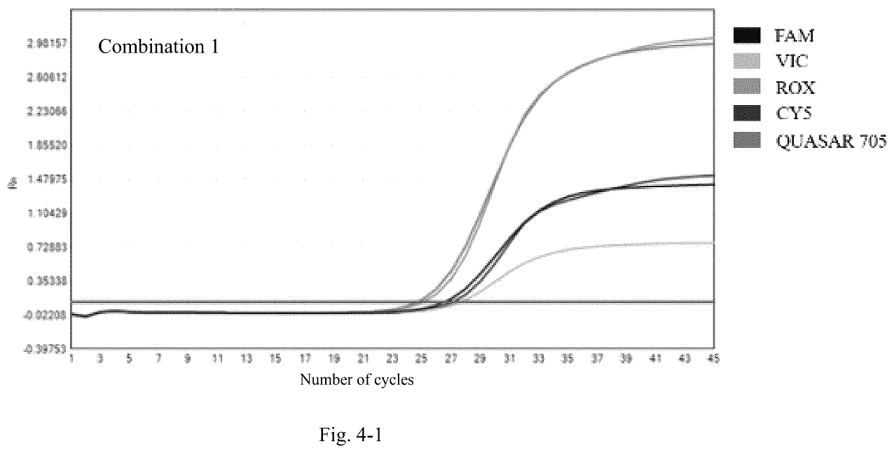

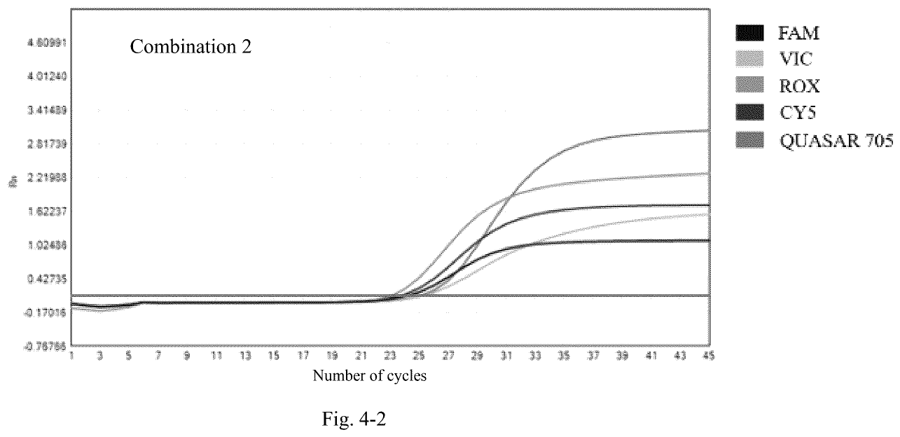

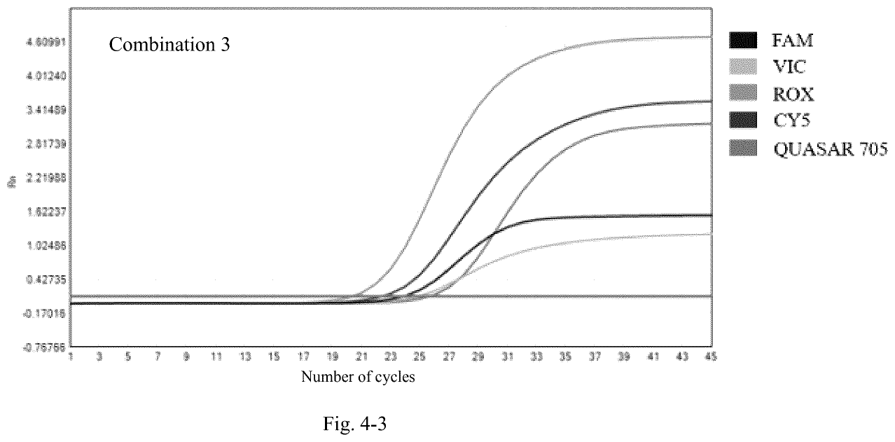

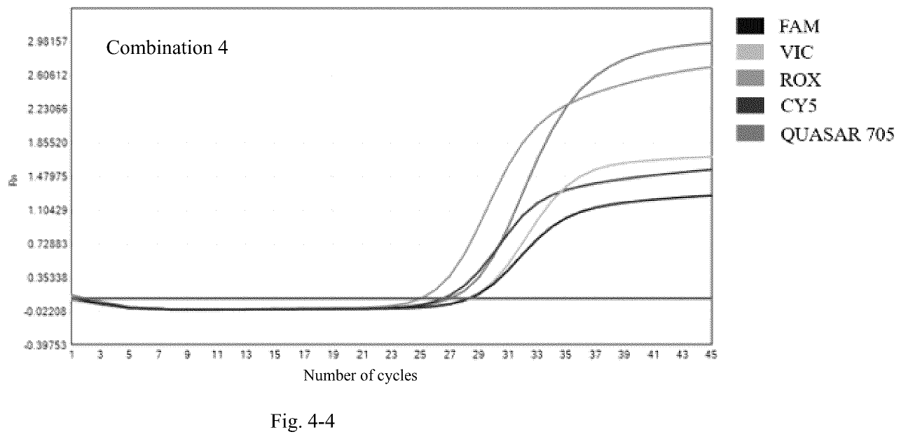

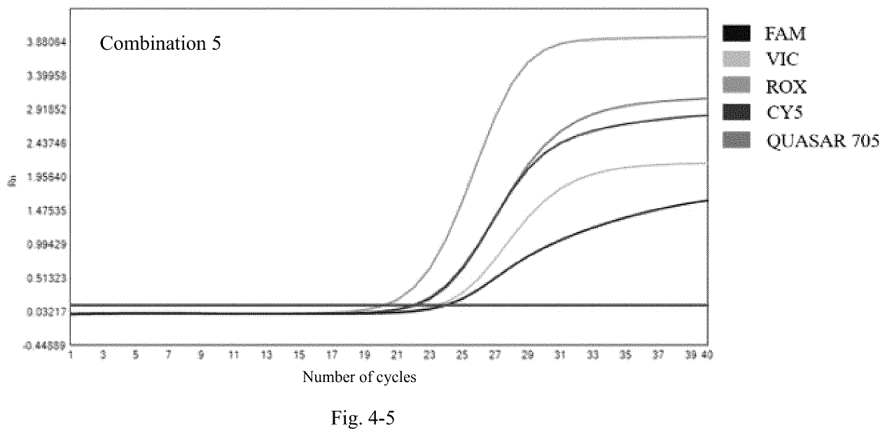

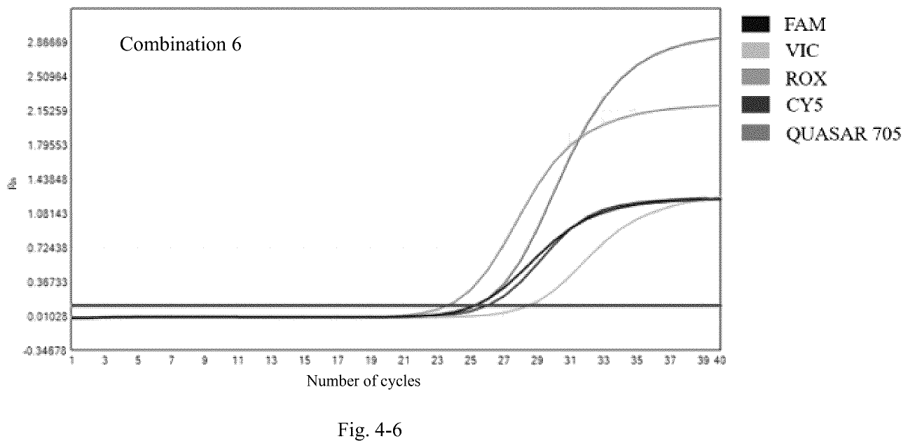

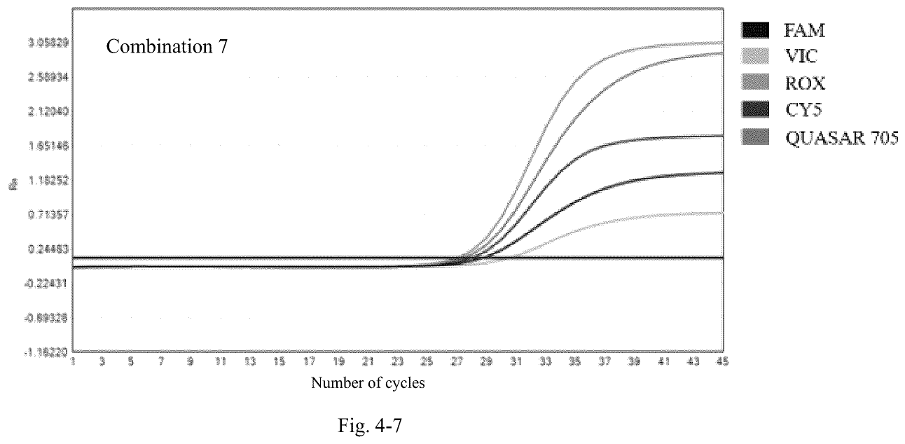

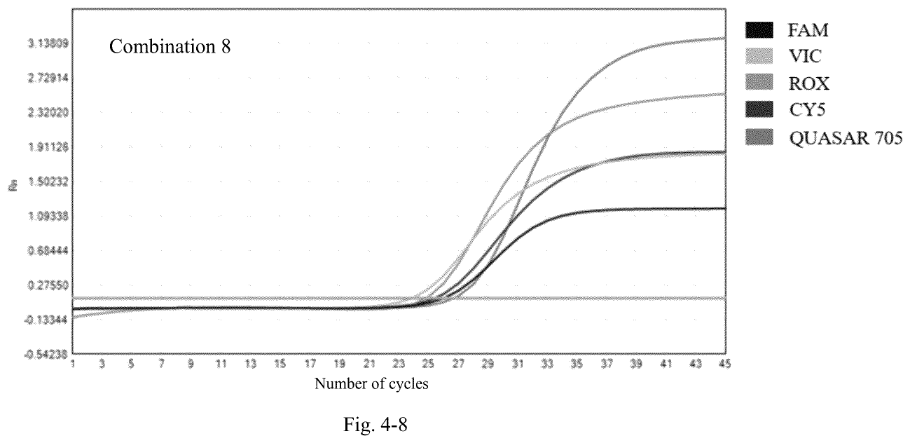

The standards of 32 kinds of target microorganisms were subjected to PCR quantification. Using a nucleic acid protectant (0.1-10 mmol/L TCEP), the standards were diluted to a concentration of 5×10⁹ copies/mL. Equal volumes of the diluted standards were mixed according to combinations 1 to 8, resulting in eight tubes of standards (S1-S8). 10-fold gradient dilutions were performed on S1-S8 to obtain a series of concentrations, such that the concentration of each target microorganism had 4 consecutive concentrations in the following concentration gradient: 1×10⁹, 1×10⁸, 1×10⁷, 1×10⁶, 1×10⁵, 1×10⁴, 1×10³ copies/mL. the above standards were subjected to fluorescence quantitative PCR amplification following the above method. According to the Ct values from the detection results and Log values of concentrations of the 32 target microbial standards, a standard curve was fitted for each target microorganism. In this example, combinations 1, 3, and 6 are used for demonstration as examples; their standard curves are shown in FIG. 1 (including FIGS. 1-1 to 1-4), FIG. 2 (including FIGS. 2-1 to 2-4), and FIG. 3 (including FIGS. 3-1 to 3-4), respectively.

### (2) Quantification of Genital Tract Microorganisms

According to the standard curves plotted using the above different quantitative standards and the Ct values from the detection results of each target microorganism, the quantification of genital tract microorganisms was performed.

### 2. Microecological Evaluation

Since vaginitis is caused by mixed infections of multiple bacteria, involving a decrease in lactobacilli and an increase in anaerobic bacteria, analysis needs to be conducted by comprehensively considering the quantification proportion of relevant target microorganisms in the sample. Based on the quantitative detection results of 32 kinds of genital tract microorganisms obtained via the above detection method, a simple, rapid, and highly accurate microecological evaluation system was constructed. This system mainly includes evaluation of bacterial vaginitis, evaluation of aerobic vaginitis, evaluation of vulvovaginal candidiasis (VVC), evaluation of trichomoniasis vaginalis (TV), evaluation of sexually transmitted infection (STI), and evaluation of HPV. The specific methods are as follows:

### (1) Genital Tract Microecological Evaluation:

Vaginal microecology is assessed according to the quantification results of 4 vaginal lactobacillus groups: Lactobacillus crispatus, Lactobacillus jensenii, Lactobacillus gasseri, and Lactobacillus iners, where when Lactobacillus crispatus accounts for a relatively high proportion, a result is “normal flora, CSTI”; when Lactobacillus jensenii accounts for a relatively high proportion, a result is “normal flora, CSTV”; when Lactobacillus gasseri accounts for a relatively high proportion, a result is “normal flora, CSTII”; when Lactobacillus iners accounts for a relatively high proportion, a result is “decreased flora function, CSTIII”; and when a proportion of pathogenic microorganisms of bacterial vaginitis (BV) or aerobic vaginitis (AV) is higher than that of the lactobacillus groups, a result is “flora imbalance.”

### (2) Genital Tract Disease Evaluation

1) Evaluation of bacterial vaginitis (BV): obtaining a P value (BV) by dividing a sum of concentrations of Lactobacillus crispatus, Lactobacillus jensenii, Lactobacillus gasseri, and Lactobacillus iners by a sum of concentrations of Gardnerella vaginalis, Prevotella, Mobiluncus, Bacteroides, Atopobium vaginae, Megasphaera, BVAB2, Ureaplasma urealyticum, and Mycoplasma hominis, P value (BV)=concentrations of Lactobacillus crispatus, Lactobacillus jensenii, Lactobacillus gasseri, and Lactobacillus iners/concentrations of Gardnerella vaginalis, Prevotella, Mobiluncus, Bacteroides, Atopobium vaginae, Megasphaera, BVAB2, Ureaplasma urealyticum, and Mycoplasma hominis; where if the P value (BV)≤1, a result is determined as bacterial vaginitis positive; and if the P value (BV)>1, a result is determined as bacterial vaginitis negative.

Although there are various kinds of vaginal lactobacilli (such as Lactobacillus reuteri, Lactobacillus plantarum, Lactobacillus fermentum, etc.), studies by the research team of the present invention have found that accurate assessment of vaginal microecology can be achieved simply through 4 microorganisms: Lactobacillus crispatus, Lactobacillus jensenii, Lactobacillus gasseri, and Lactobacillus iners. Moreover, based on these 4 vaginal lactobacilli, assessment of bacterial vaginitis (BV) and aerobic vaginitis (AV) can be performed by using content relationships or ratio relationships of the vaginal lactobacilli with other pathogenic bacteria. Compared with the traditional microscopic examination method, this method is more convenient, accurate, efficient, and cost-saving.

According to the evaluation method of bacterial vaginitis (BV) provided in this example, determination can be made simply through nucleic acid detection and quantification results of 4 vaginal lactobacilli and 9 pathogenic microorganisms associated with bacterial vaginitis. Specifically, whether a result is bacterial vaginitis positive is assessed according to whether the proportion of the 9 pathogenic microorganisms is higher than that of the 4 lactobacillus groups. The result is more accurate and reliable compared with the traditional microscopic examination method.
2) Evaluation of aerobic vaginitis (AV): obtaining a P value (AV) by dividing the sum of concentrations of Lactobacillus crispatus, Lactobacillus jensenii, Lactobacillus gasseri, and Lactobacillus iners by a sum of concentrations of Group A Streptococcus, Group B Streptococcus, Staphylococcus aureus, Escherichia coli, Enterococcus faecalis, Streptococcus anginosus, and Klebsiella pneumoniae, or P value (AV)-concentrations of Lactobacillus crispatus, Lactobacillus jensenii, Lactobacillus gasseri, and Lactobacillus iners/concentrations of Group A Streptococcus, Group B Streptococcus, Staphylococcus aureus, Escherichia coli, Enterococcus faecalis, Streptococcus anginosus, and Klebsiella pneumoniae; where if the P value (AV)≤1, a result is determined as aerobic vaginitis positive; and if the P value (AV)>1, a result is determined as aerobic vaginitis negative.

According to the evaluation method of aerobic vaginitis (AV) provided in this example, determination can be made simply through nucleic acid detection and quantification results of 4 vaginal lactobacilli and 7 pathogenic microorganisms associated with aerobic vaginitis. Specifically, whether a result is aerobic vaginitis positive is assessed according to whether the proportion of the 7 pathogenic microorganisms is higher than that of the 4 lactobacillus groups. The result is more accurate and reliable compared with the traditional microscopic examination method.
3) Evaluation of vulvovaginal candidiasis (VVC): if Candida spp, and/or Candida albicans is detected as positive, a result is determined as VVC positive; and if Candida spp, and/or Candida albicans is detected as negative, a result is determined as VVC negative.
4) Evaluation of trichomoniasis vaginalis (TV): if Trichomonas vaginalis is detected as positive, a result is determined as TV positive; and if Trichomonas vaginalis is detected as negative, a result is determined as TV negative.
5) Evaluation of sexually transmitted infection (STI): if one or more of Neisseria gonorrhoeae, Treponema pallidum, Chlamydia trachomatis, Mycoplasma genitalium, herpes simplex virus type I, and herpes simplex virus type II are detected as positive, a result is determined as STI positive; and if one or more of Neisseria gonorrhoeae, Treponema pallidum, Chlamydia trachomatis, Mycoplasma genitalium, herpes simplex virus type I, and herpes simplex virus type II are detected as negative, a result is determined as STI negative.
6) Evaluation of HPV: if any one or more of HPV16, HPV18, and other high-risk HPV types are detected as positive, a result is determined as HPV positive; and if any one or more of HPV16, HPV18, and other high-risk HPV types are detected as negative, a result is determined as HPV negative.

In a complex vaginal environment, there are many types of microorganisms. The evaluation of the microecological environment is closely related to subsequent treatment. Through extensive experiments, this example found: the positive or negative status of vaginal inflammation can be directly determined by using the relationship or concentration ratio between 4 types of lactobacilli (beneficial microorganisms) and other limited pathogenic bacteria. This is consistent with actual traditional microbial culture methods, thus forming a diagnostic system from nucleic acid amplification to the determination of inflammation as negative or positive.

### Example 3. Simulated Sample Detection and Performance Analysis

### 1. Simulated Sample Detection Results

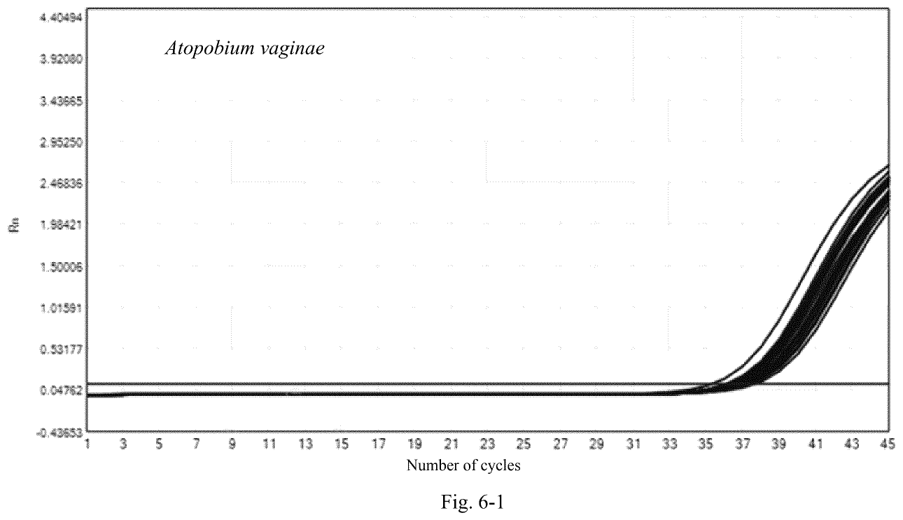

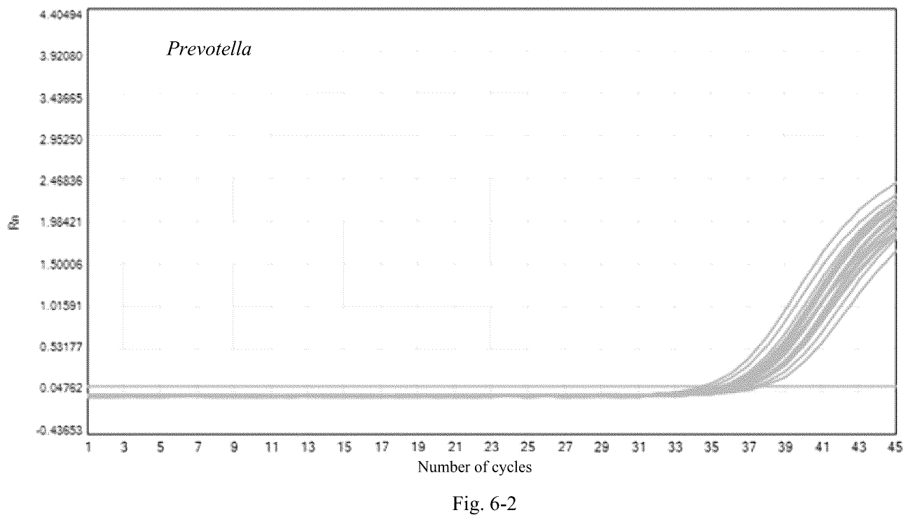

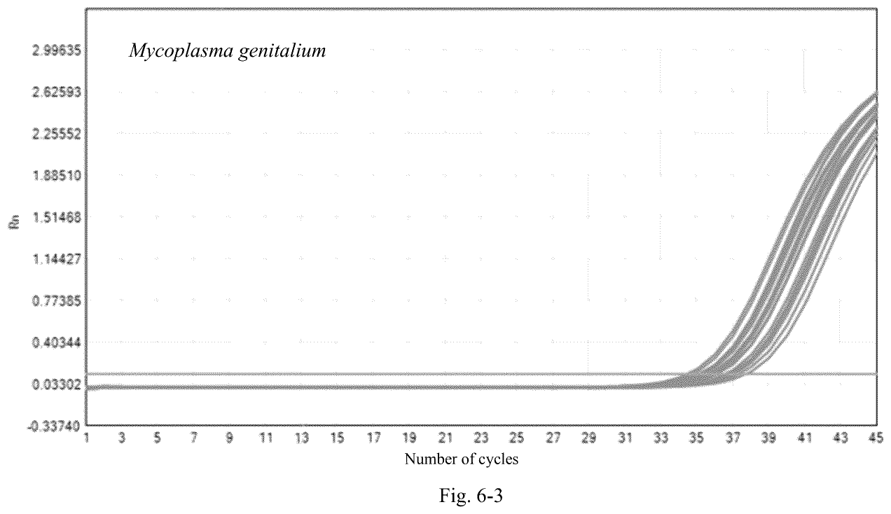

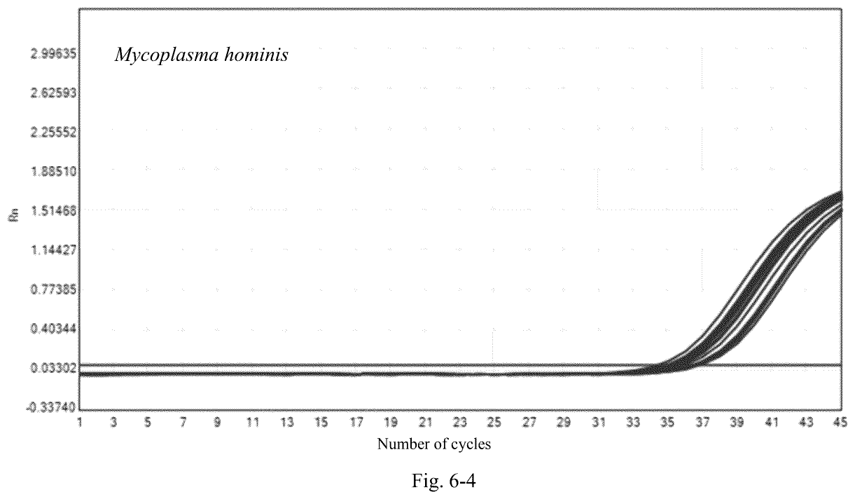

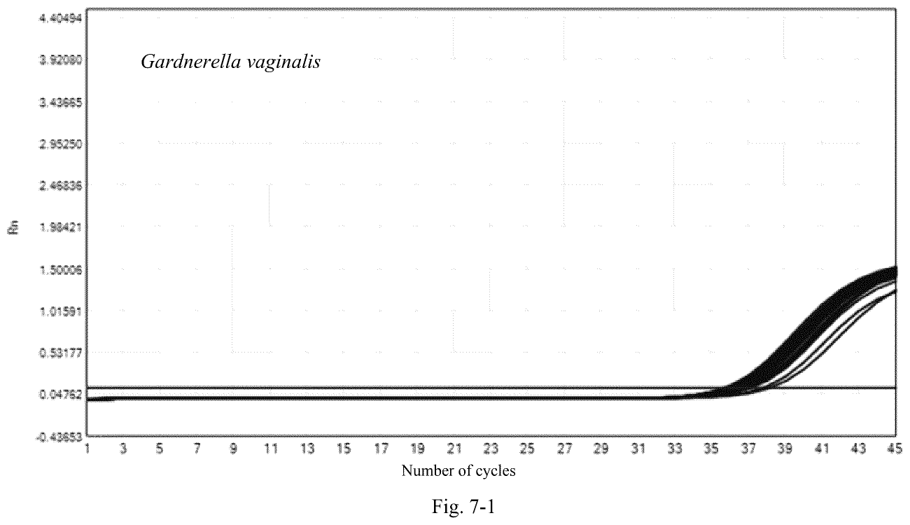

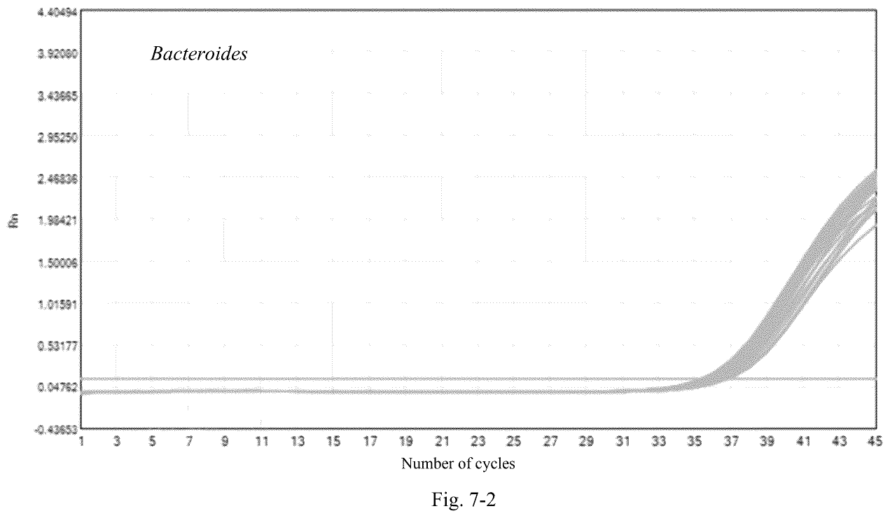

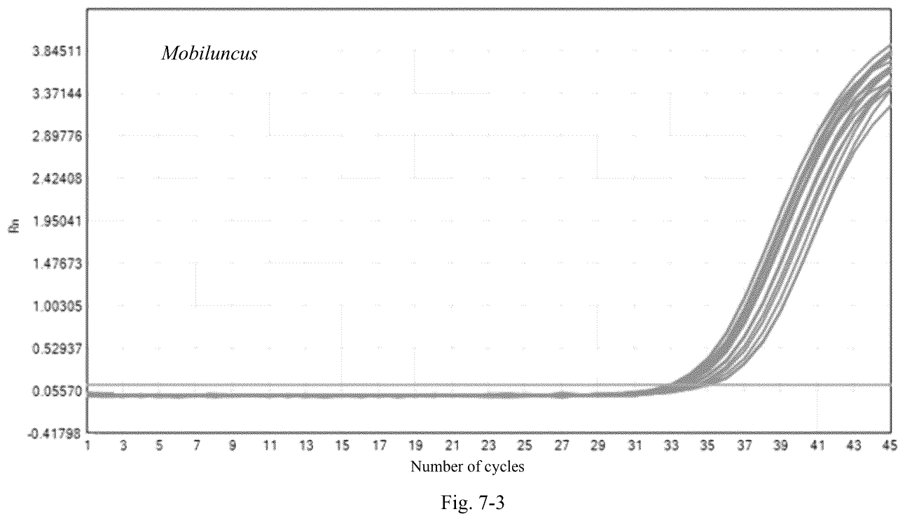

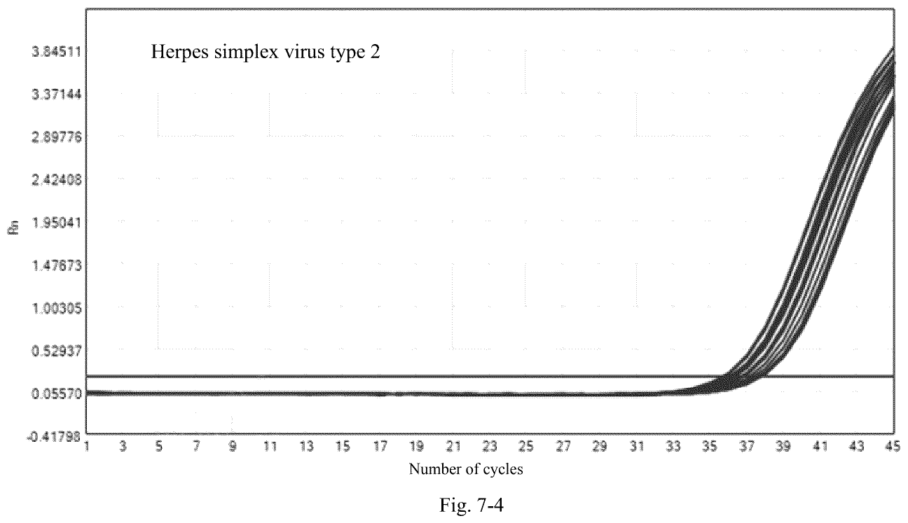

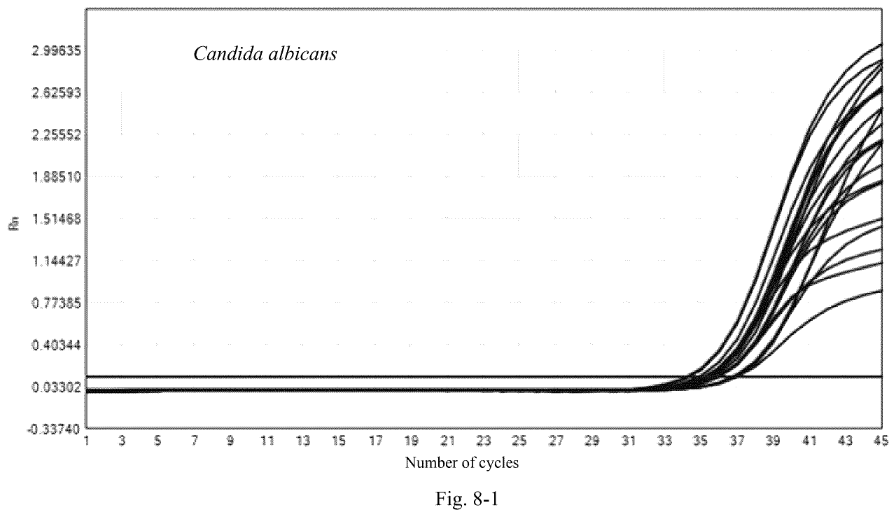

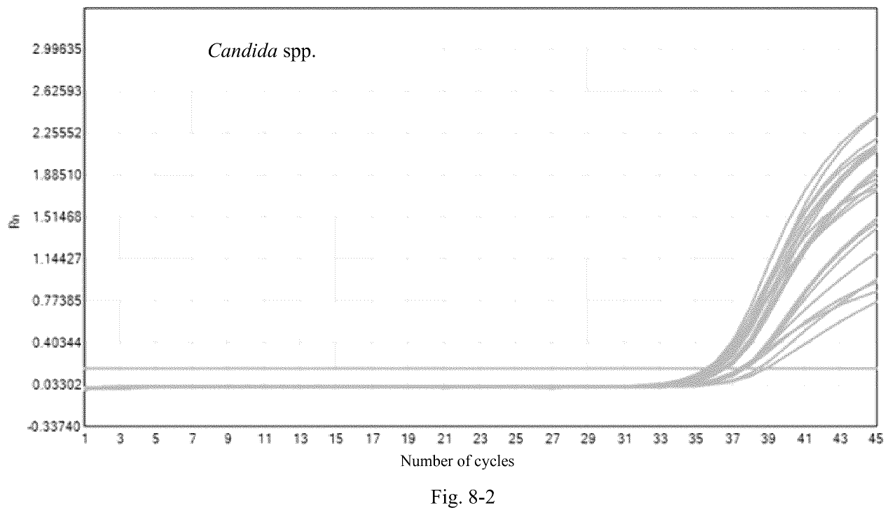

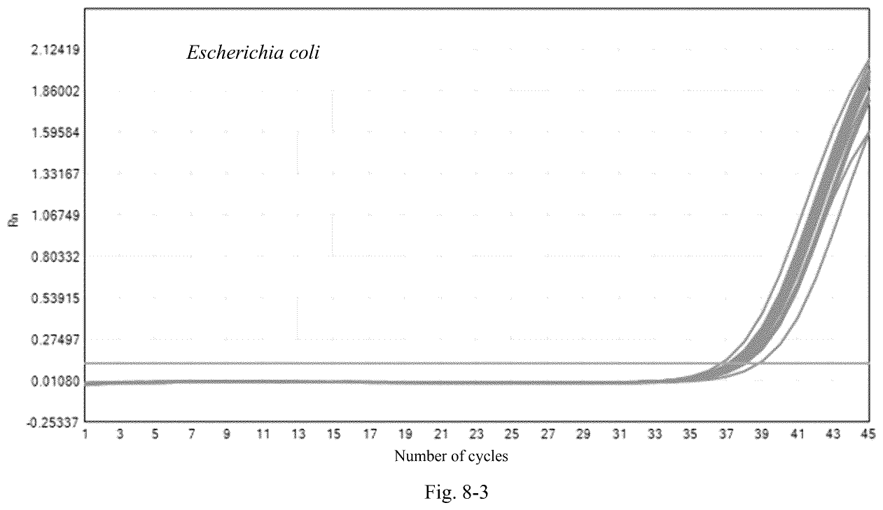

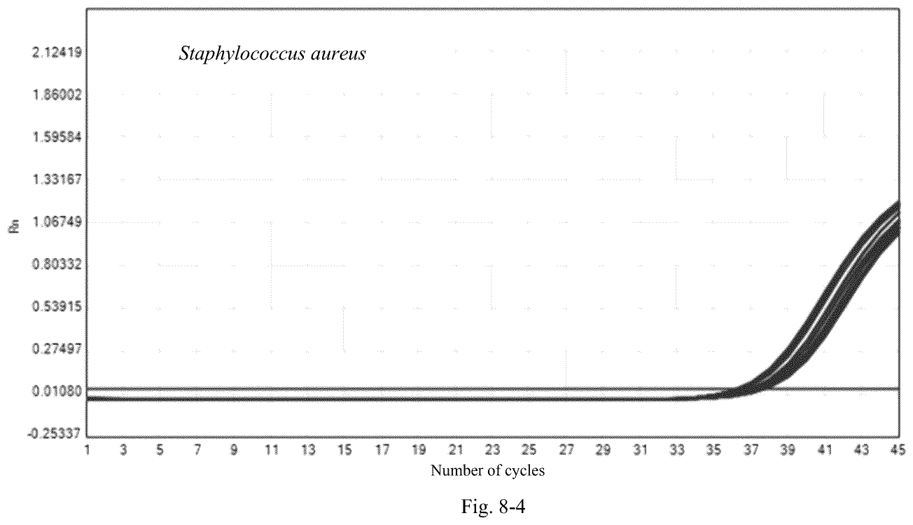

The simulated samples 1-8 used for detection are as follows: simulated sample 1 is a mixture of Lactobacillus crispatus, Lactobacillus jensenii, Lactobacillus gasseri, Lactobacillus iners, and an internal standard plasmid; simulated sample 2 is a mixture of Atopobium vaginae, Prevotella, Mycoplasma genitalium, Mycoplasma hominis, and an internal standard plasmid; simulated sample 3 is a mixture of Gardnerella vaginalis, Bacteroides, Mobiluncus, herpes simplex virus type 2, and an internal standard plasmid; simulated sample 4 is a mixture of Candida albicans, Candida tropicalis, Escherichia coli, Staphylococcus aureus, and an internal standard plasmid; simulated sample 5 is a mixture of Neisseria gonorrhoeae, Chlamydia trachomatis, Ureaplasma urealyticum, Trichomonas vaginalis, and an internal standard plasmid; simulated sample 6 is a mixture of HPV type 16, HPV type 18, other high-risk HPV types, Group B Streptococcus, and an internal standard plasmid; simulated sample 7 is a mixture of Megasphaera, BVAB2, Treponema pallidum, herpes simplex virus type 1, and an internal standard plasmid; and simulated sample 8 is a mixture of Streptococcus anginosus, Enterococcus faecalis, Klebsiella pneumoniae, Group A Streptococcus, and an internal standard plasmid. The content of each microorganism in the simulated samples was formulated separately as needed. The detection results of simulated samples 1-8 are shown in FIGS. 4-1 to 4-8. The results show that the detection method provided in this example can distinguish the microorganisms within the detection range well.

### 2. Sensitivity Analysis

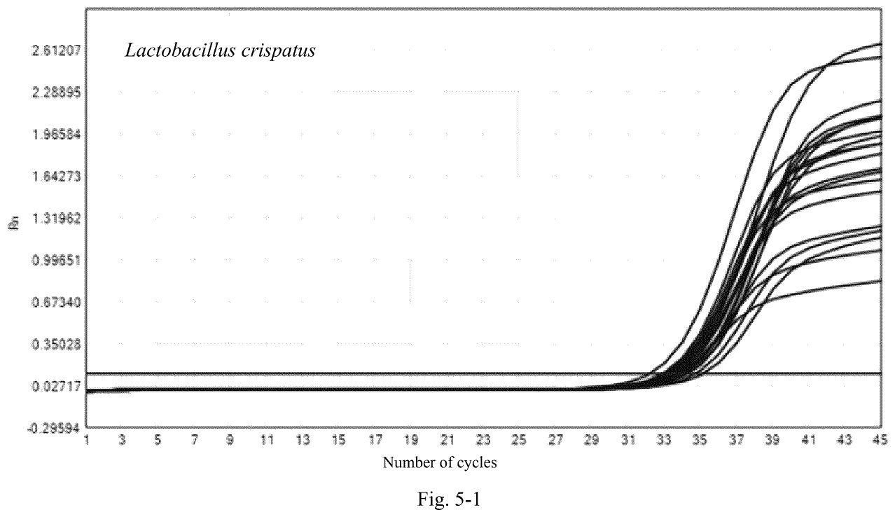

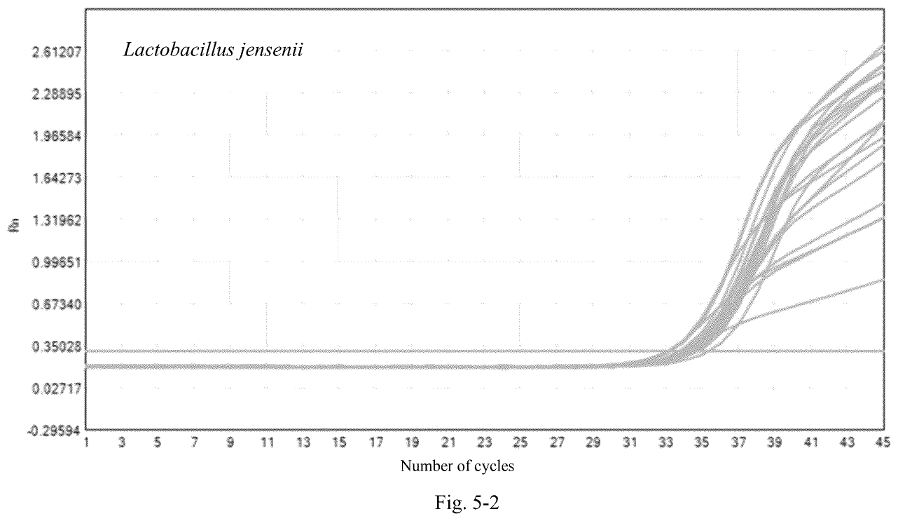

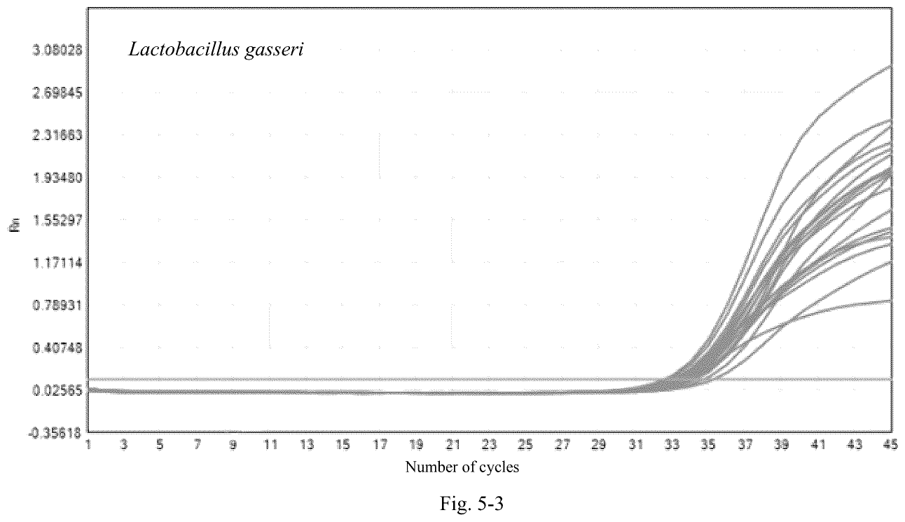

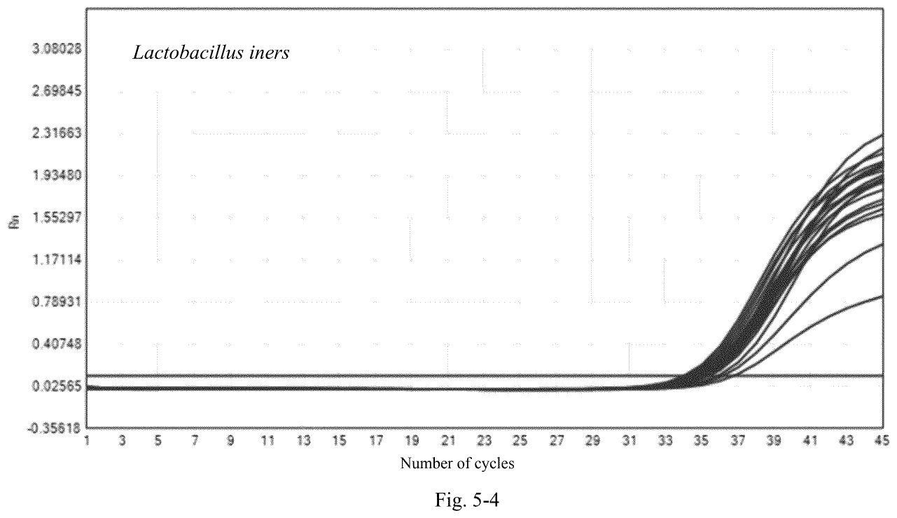

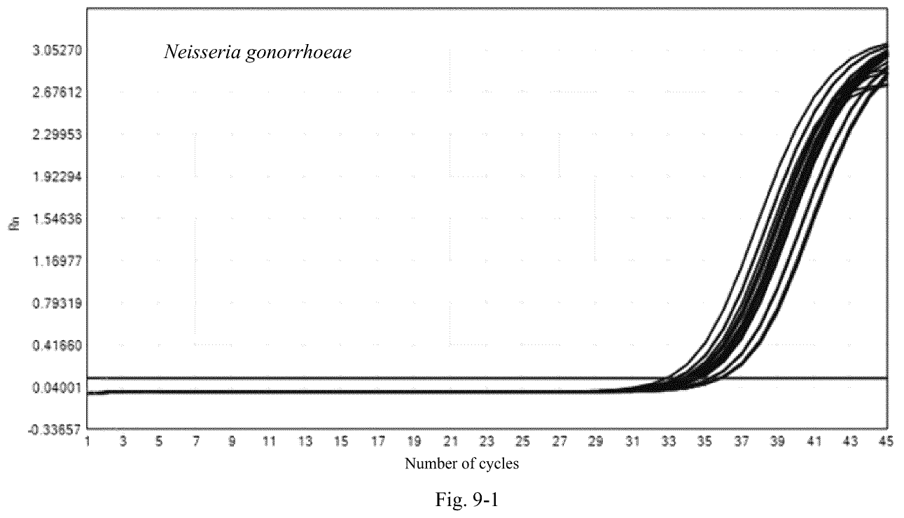

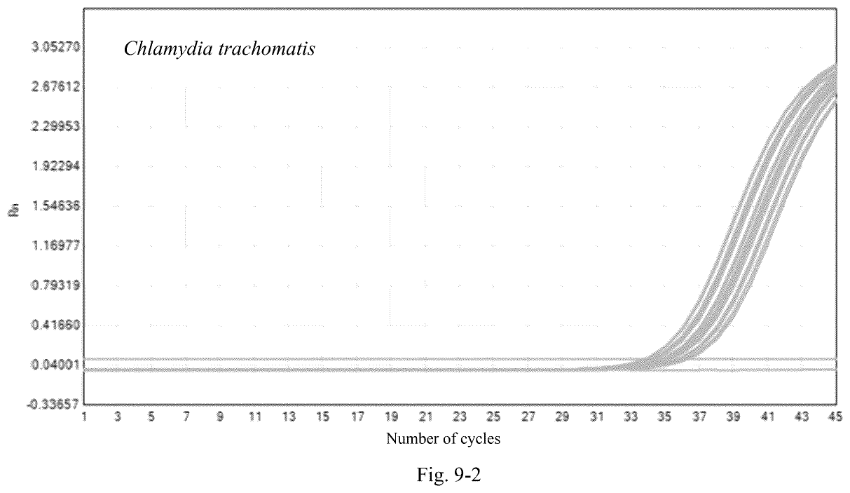

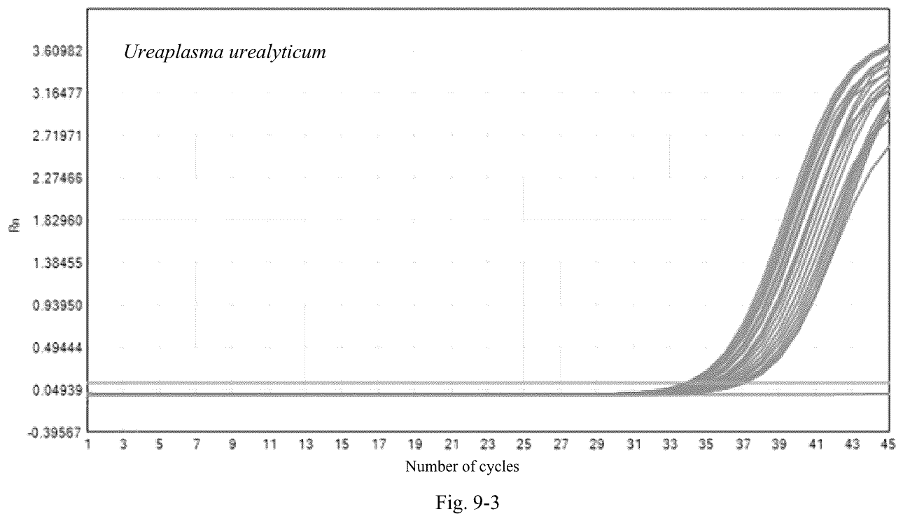

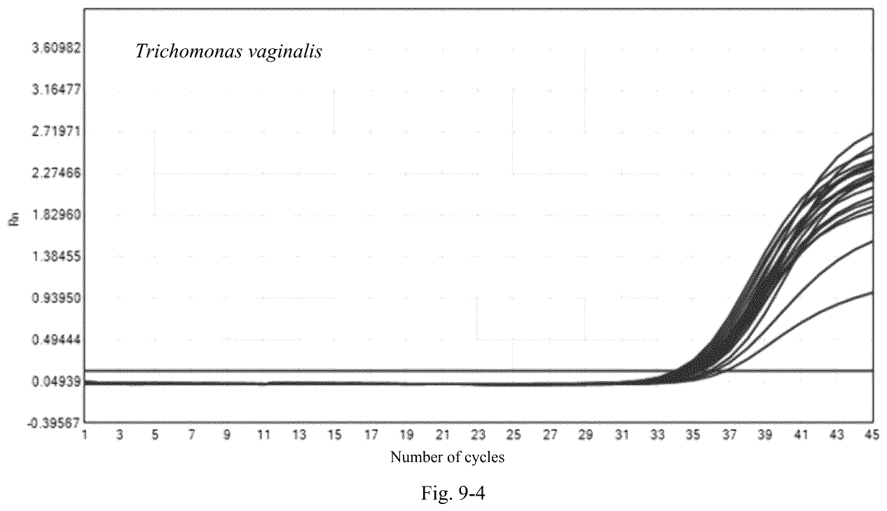

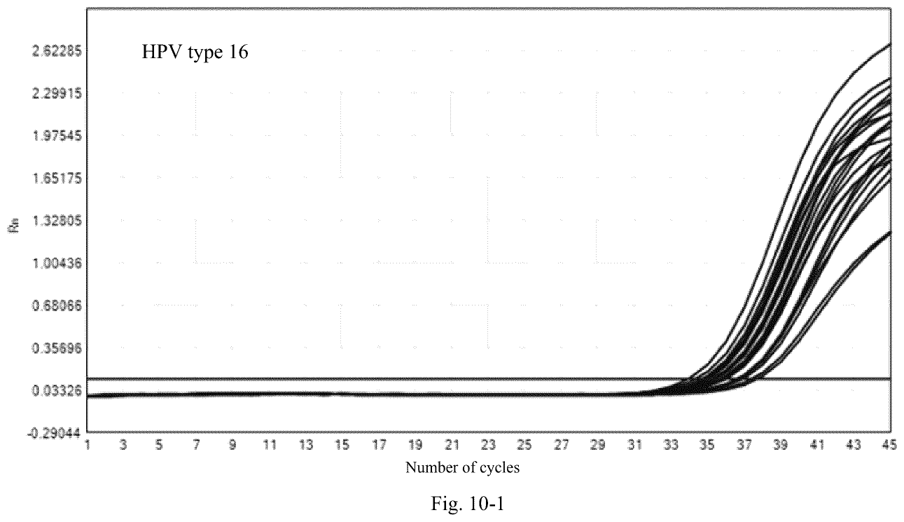

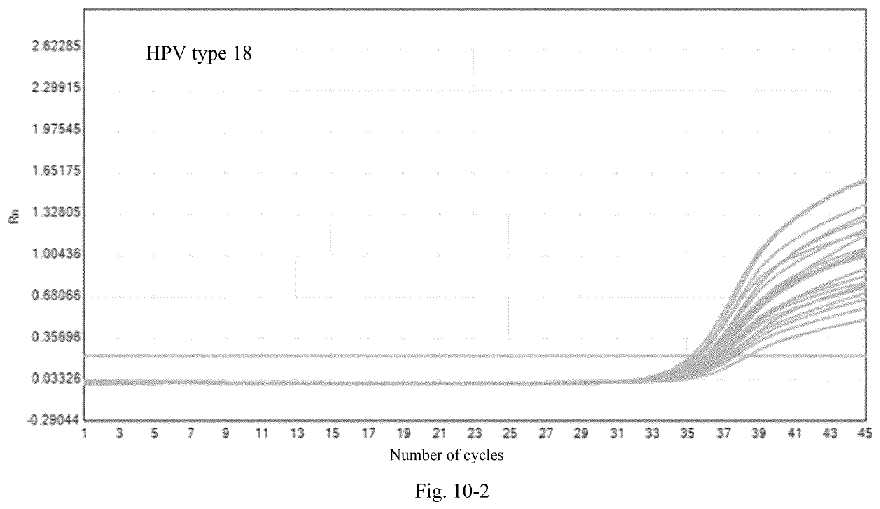

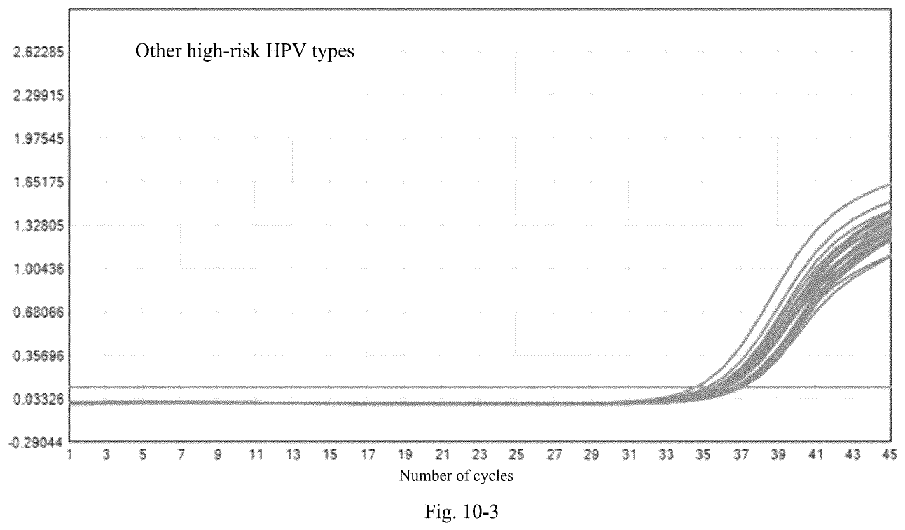

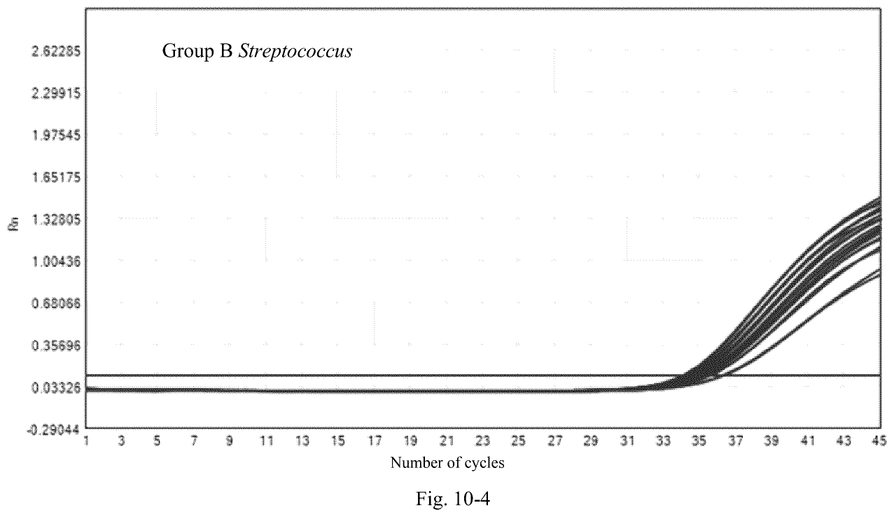

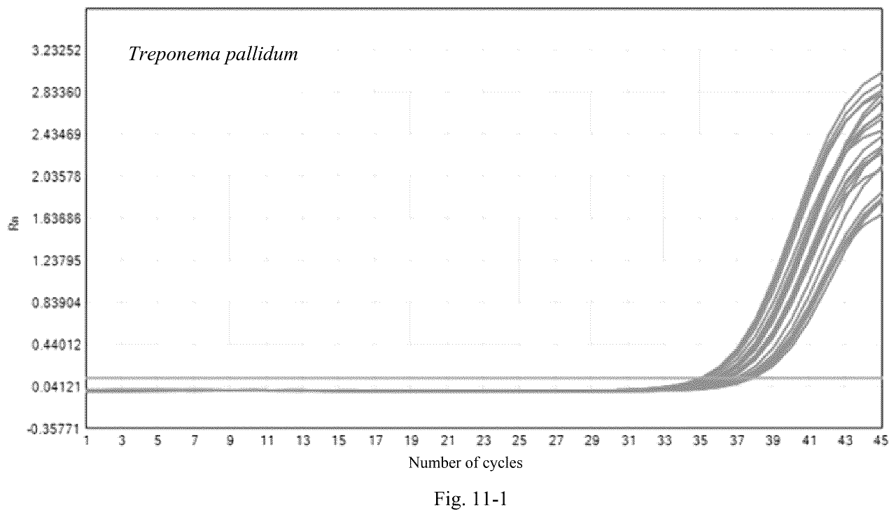

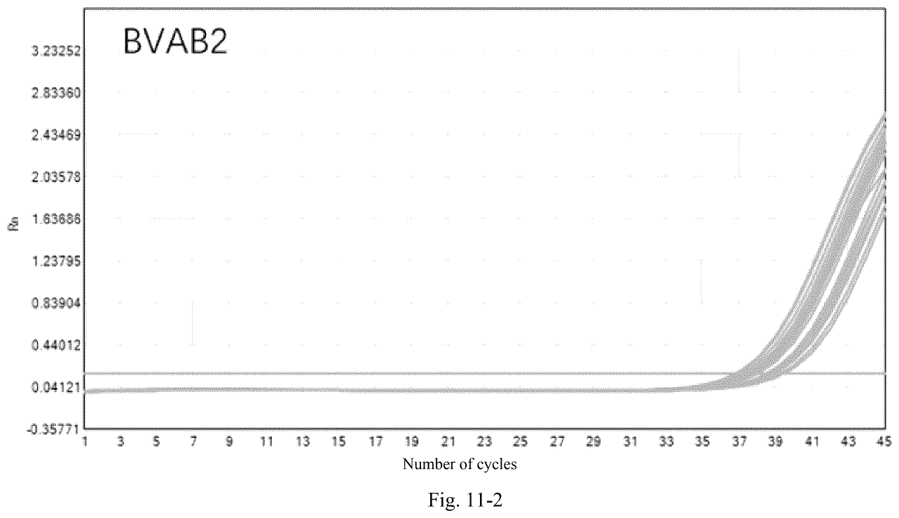

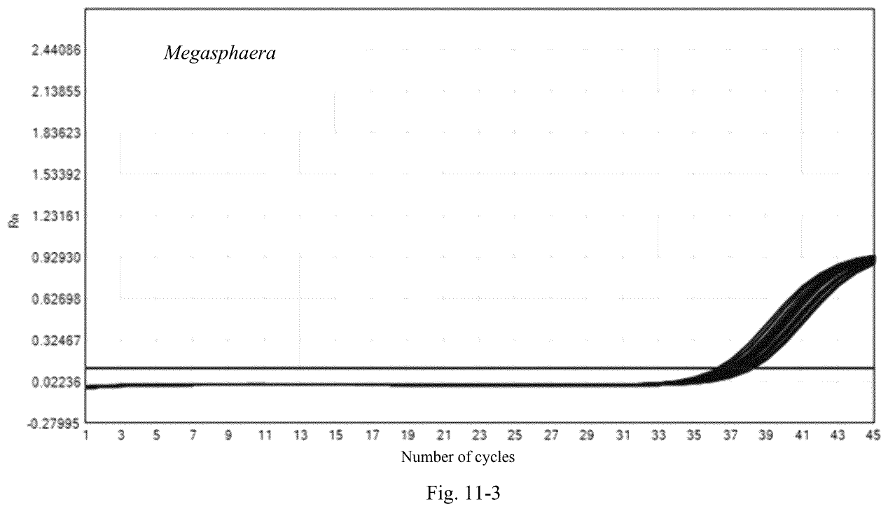

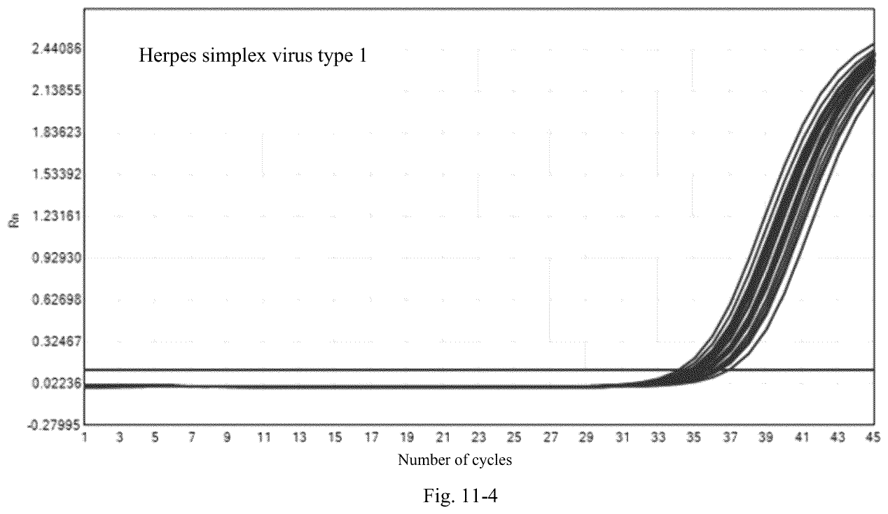

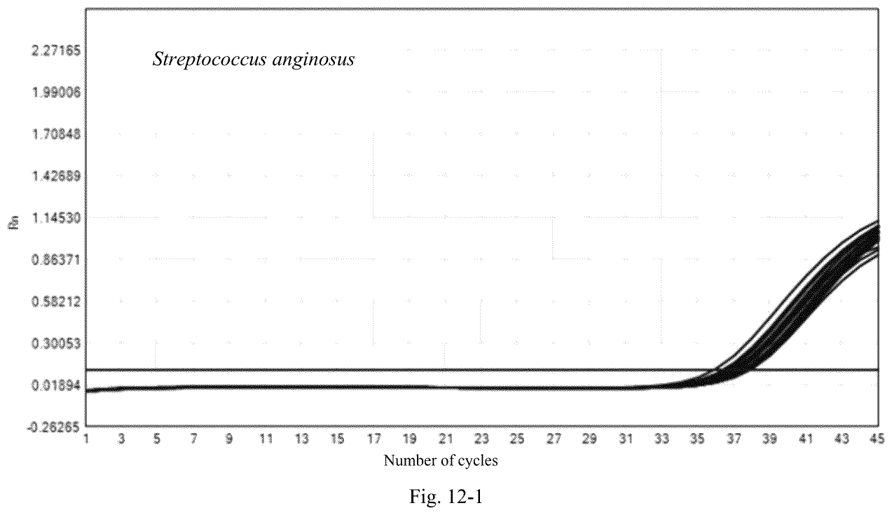

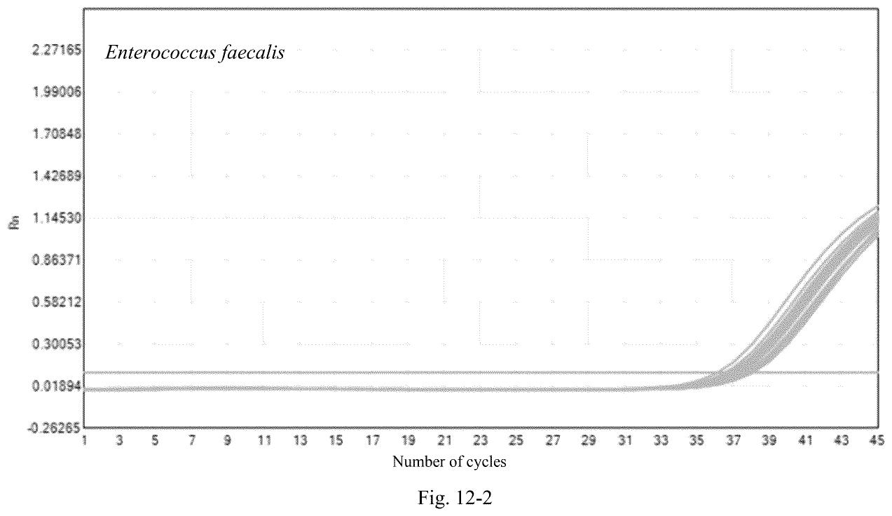

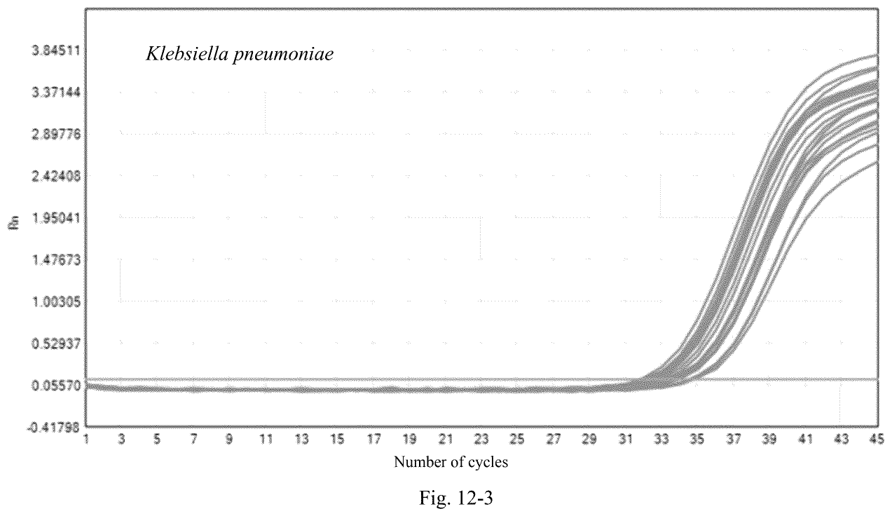

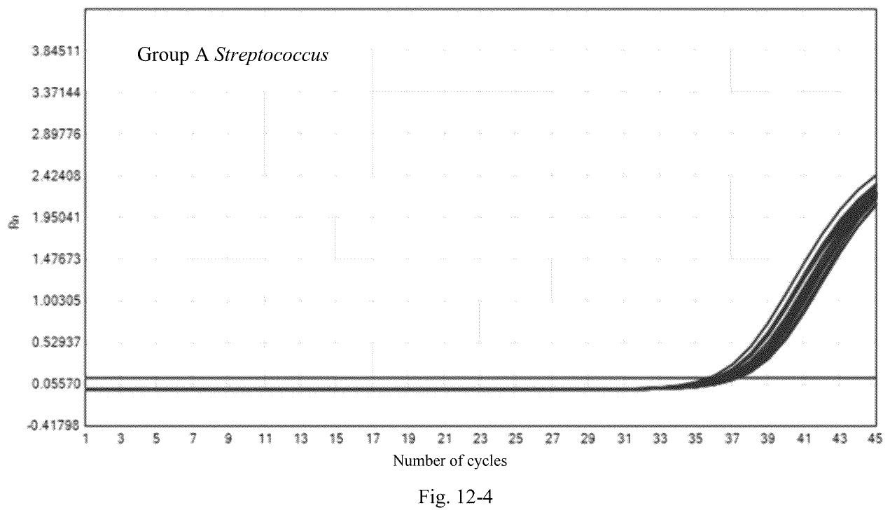

The primer-probe combinations in Table 4 were used to test the sensitivity of each target. The quantified clinical samples were serially diluted to concentrations of 2,000, 1,000, 500, 250, and 100 copies/mL, with 3 replicate detections performed for each concentration. The lowest concentration at which all targets were detected was subjected to 20 replicate detections. The results are shown in FIG. 5 (including FIGS. 5-1 to 5-4) to 12 (including FIGS. 12-1 to 12-4). The results show that the combination can detect samples at 100-2,000 copies/mL, indicating that the combination of the present invention has a sensitivity of 100 copies/mL for different target microorganisms.

### 3. Specificity Analysis

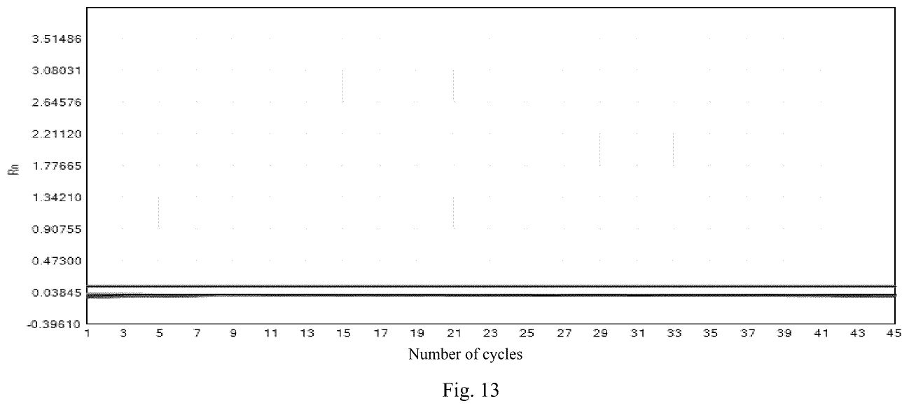

The target genes of microorganisms within the detection range of the primer-probe combinations in Table 4 were subjected to sequence alignment analysis with pathogens outside the detection range that infect the same site or cause similar symptoms (including BVAB1, varicella-zoster virus, HPV6, HPV11, Acinetobacter baumannii, Pseudomonas aeruginosa, Mycobacterium tuberculosis, and cytomegalovirus). Additionally, the kit was used to detect high-concentration samples of these pathogens. The results are shown in FIG. 13, which show that the kit failed to detect any of these pathogens, indicating that the combination of the present invention has good specificity.

### 4. Precision Analysis

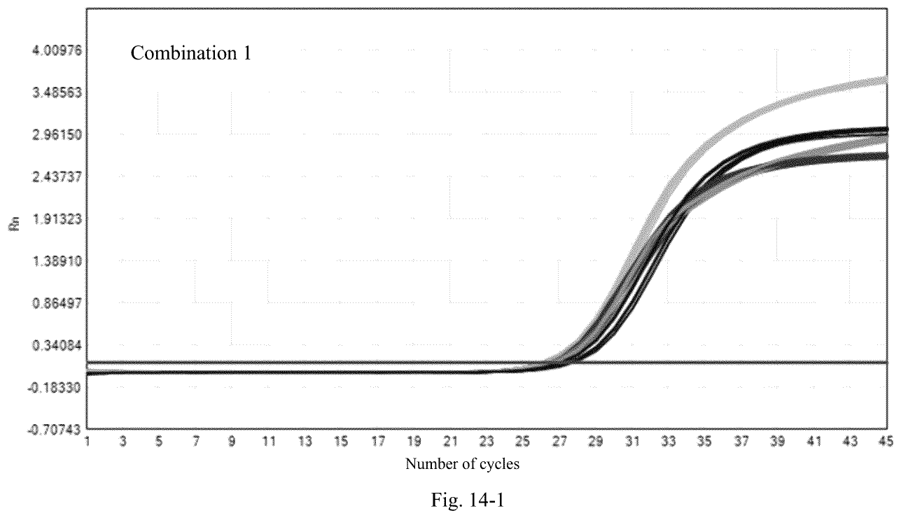

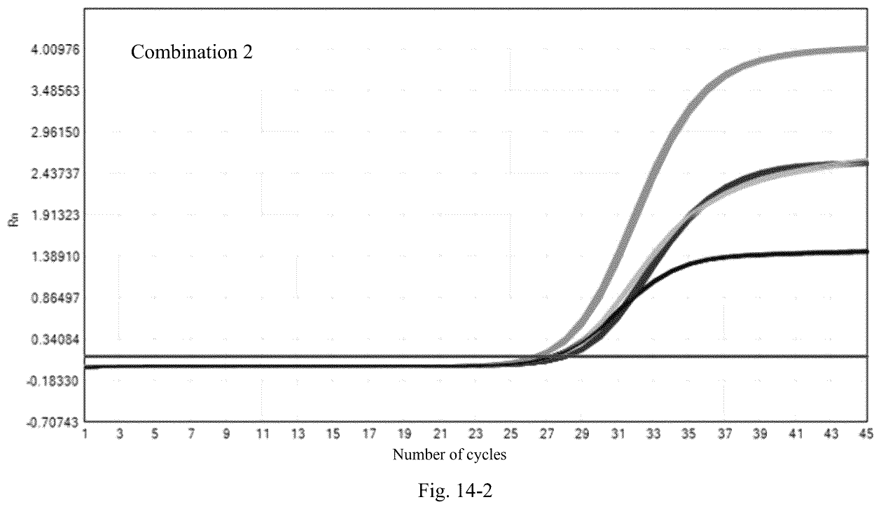

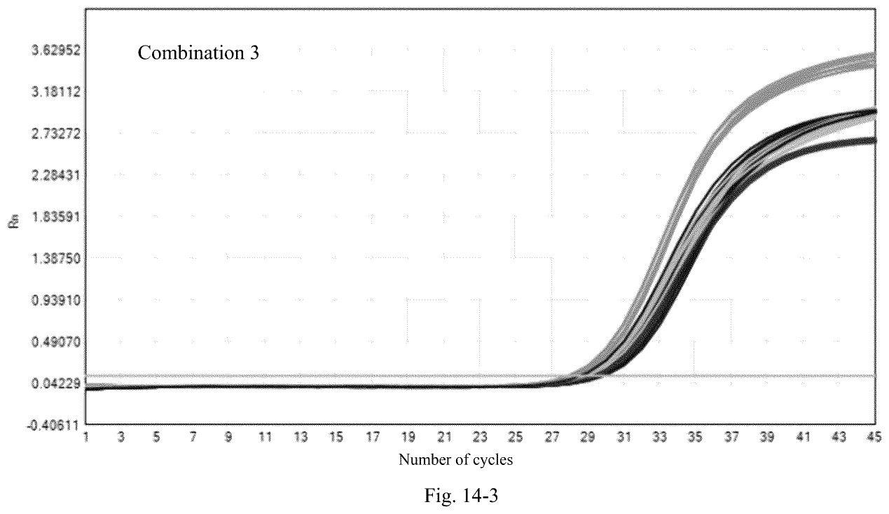

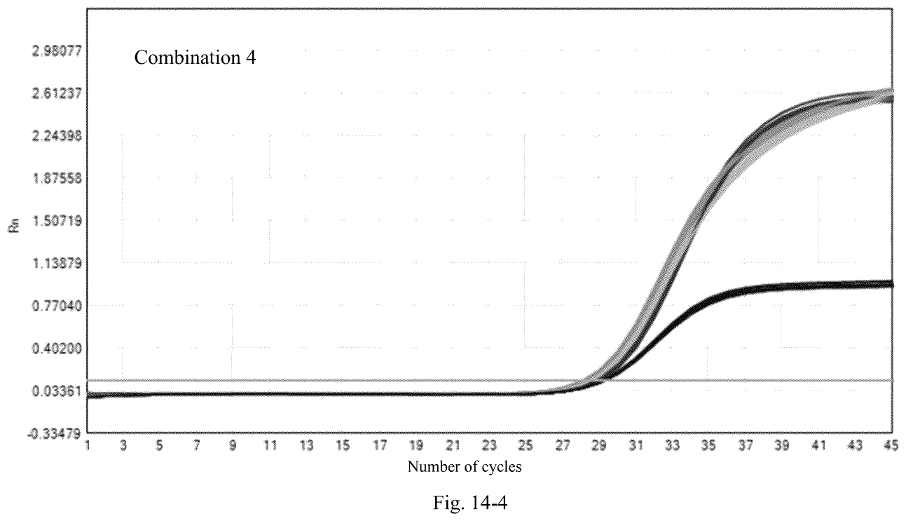

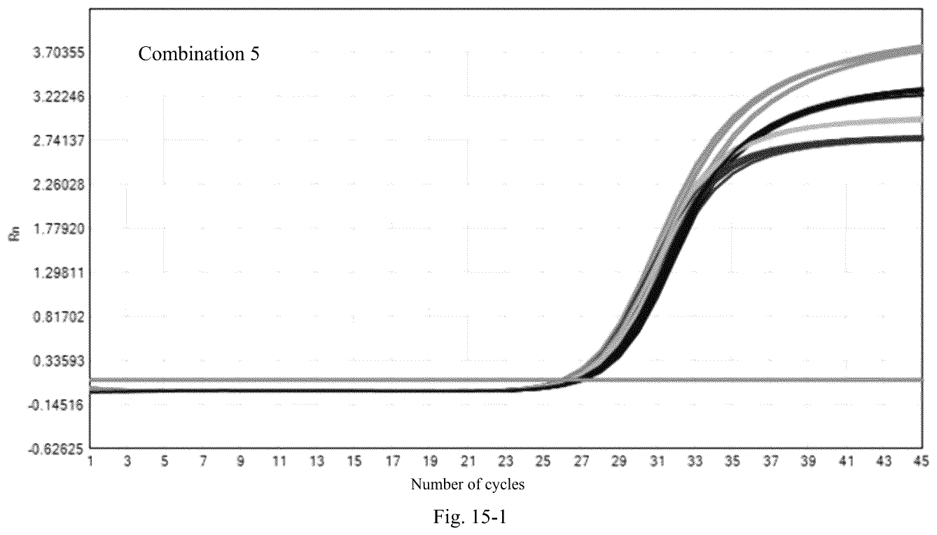

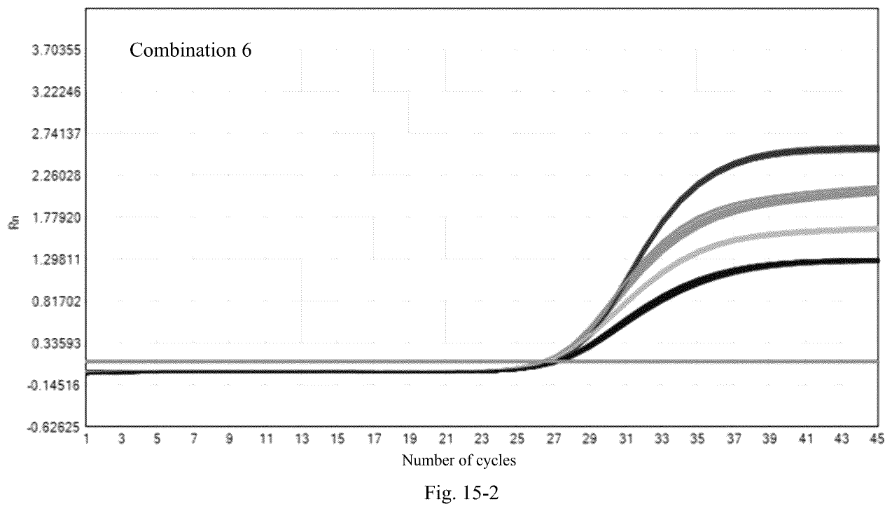

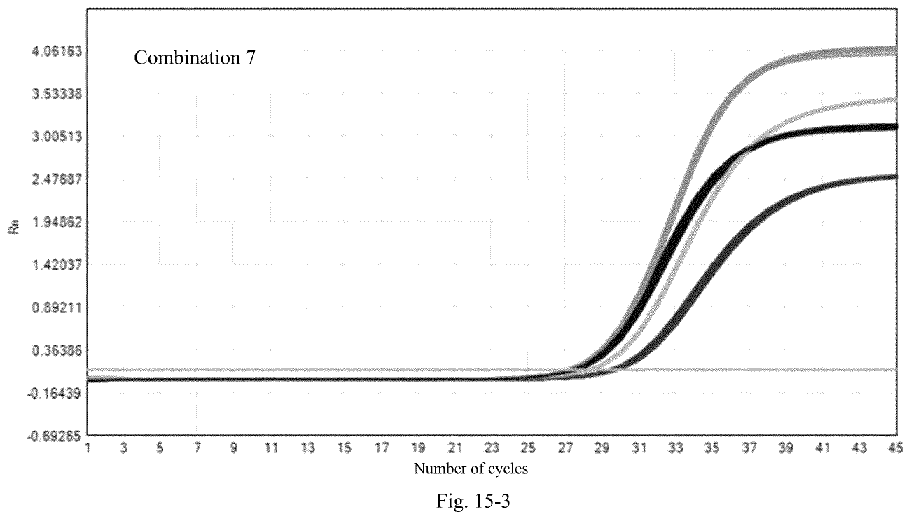

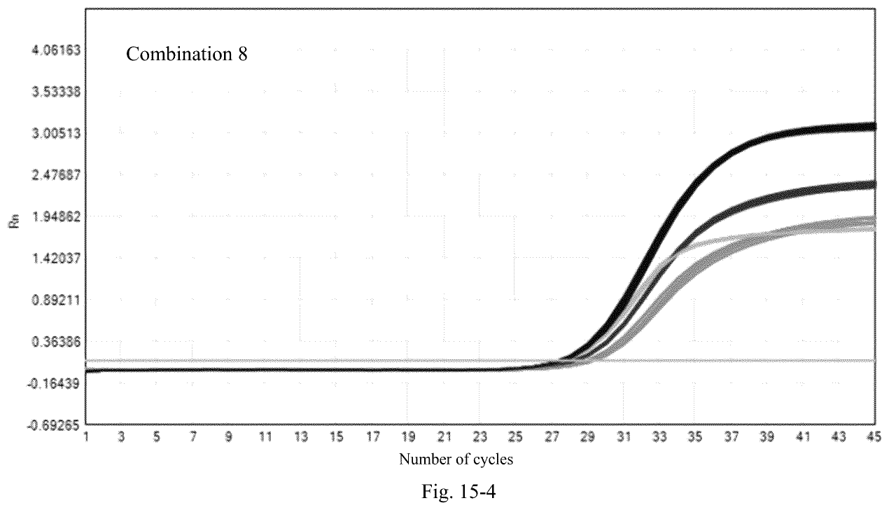

In this example, simulated samples 1-8 as described above were adopted, and 10 replicate detections were performed for each sample using the above method. The detection results are shown in FIG. 14 (including FIGS. 14-1 to 14-4) to 15 (including FIGS. 15-1 to 15-4). The coefficients of variation (CV, %) of the calculated Ct values are shown in Table 7. The results show that all CV values are <1.74%, indicating that the combination has good precision.

TABLE 7
Precision results

| | CV value | | | |
|---|---|---|---|---|
| Sample | FAM | VIC | ROX | CY5 |
| J1 | 0.24% | 0.65% | 0.31% | 0.86% |
| J2 | 0.06% | 0.42% | 1.74% | 0.63% |
| J3 | 1.48% | 0.69% | 0.60% | 0.39% |
| J4 | 0.04% | 0.07% | 0.05% | 0.41% |
| J5 | 0.39% | 1.05% | 0.28% | 0.92% |
| J6 | 0.09% | 0.07% | 0.09% | 0.28% |
| J7 | 0.15% | 0.07% | 0.08% | 0.53% |
| J8 | 0.13% | 0.08% | 0.70% | 0.20% |

### Example 4. Accuracy Verification

1. Comparison with the Gold Standard Method

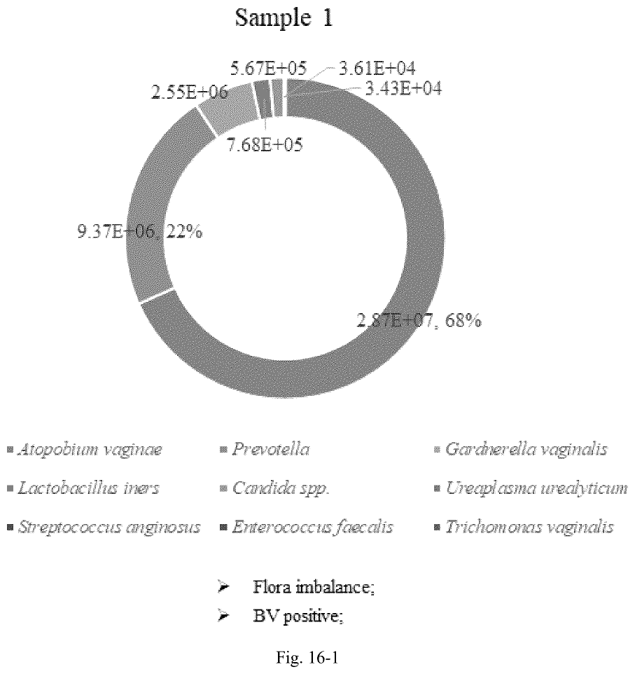

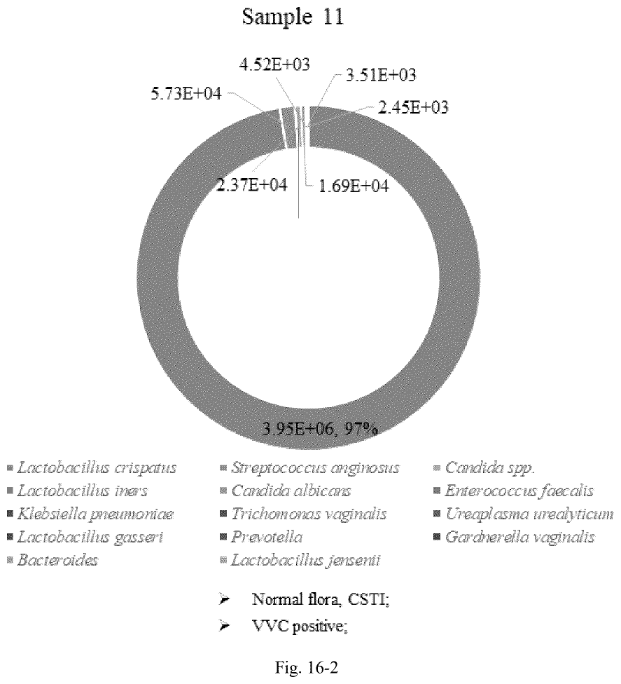

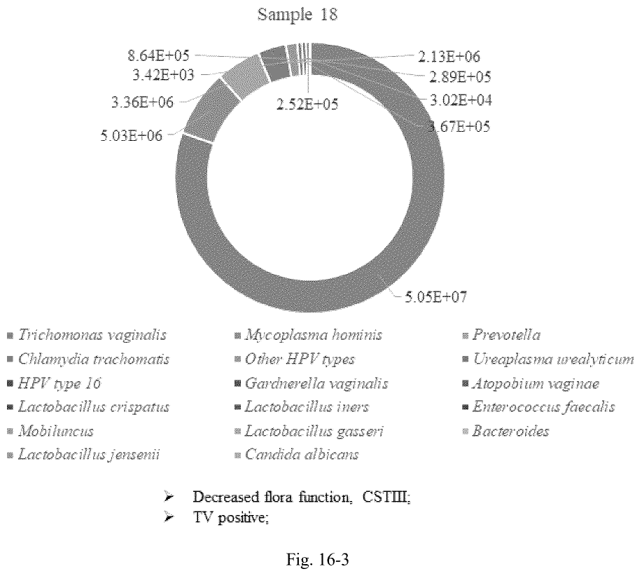

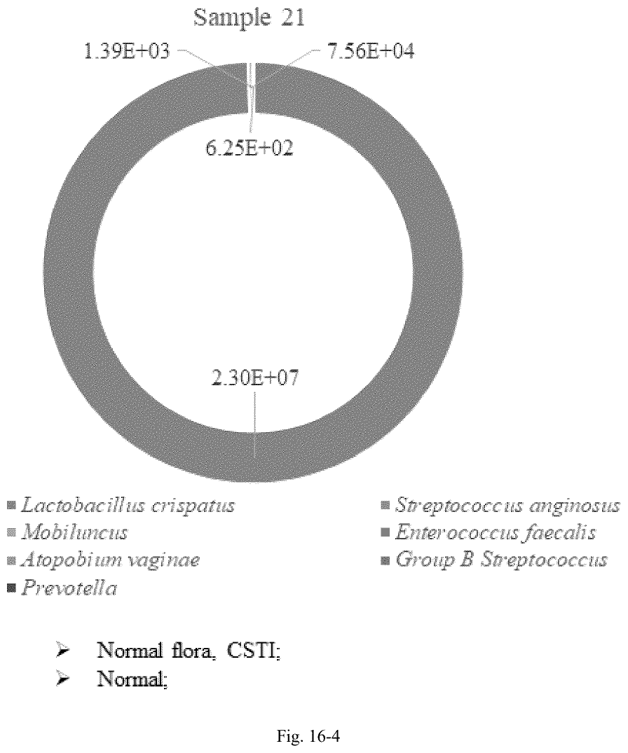

Twenty-five clinical samples diagnosed by the gold standard (Nugent comprehensive scoring method) or microscopic observation were collected from a hospital, with 10 samples diagnosed as bacterial vaginosis (BV) positive, 7 samples diagnosed as vulvovaginal candidiasis (VVC) positive, 3 samples diagnosed as trichomoniasis vaginalis (TV) positive, and 5 samples diagnosed as normal. The combination was used for detection to verify the application accuracy of the combination. The comparative detection results are shown in Table 13. In this example, sample 1, sample 11, sample 18, and sample 21 are used for demonstration as examples, and the detection results are shown in FIG. 16 (including FIGS. 16-1 to 16-4).

TABLE 13
Comparative results between the combination and the gold standard method

| | Diagnosis | | | |
|---|---|---|---|---|
| | result of | | | |
| Sample | the gold | Detection result of the present invention | | Consistent |
| number | standard | Detected microorganism | Interpretation | (Yes/No) |
| Sample | BV+ | Lactobacillus iners, Atopobium vaginae, Prevotella, | Flora | Yes |
| 1 | | Gardnerella vaginalis, Candida spp., Ureaplasma | imbalance; | |
| | | urealyticum, and Trichomonas vaginalis | BV+ | |
| Sample | BV+ | Lactobacillus crispatus, Lactobacillus jensenii, | Flora | Yes |
| 2 | | Lactobacillus iners, Atopobium vaginae, Prevotella, | imbalance; | |
| | | Mycoplasma hominis, Gardnerella vaginalis, | BV+ | |
| | | Mobiluncus, Ureaplasma urealyticum, and | | |
| | | Trichomonas vaginalis | | |
| Sample | BV+ | Lactobacillus iners, Atopobium vaginae, Prevotella, | Flora | Yes |
| 3 | | Gardnerella vaginalis, Ureaplasma urealyticum, and | imbalance; | |
| | | Trichomonas vaginalis | BV+ | |
| Sample | BV+ | Lactobacillus crispatus, Lactobacillus iners, | Flora | Yes |
| 4 | | Atopobium vaginae, Prevotella, Gardnerella | imbalance; | |
| | | vaginalis, Ureaplasma urealyticum, and | BV+ | |
| | | Trichomonas vaginalis | | |
| Sample | BV+ | Lactobacillus iners, Atopobium vaginae, Prevotella, | Flora | Yes |
| 5 | | Gardnerella vaginalis, Mobiluncus, Ureaplasma | imbalance; | |
| | | urealyticum, and Trichomonas vaginalis | BV+ | |
| Sample | BV+ | Lactobacillus crispatus, Lactobacillus iners, | Flora | Yes |
| 6 | | Atopobium vaginae, Prevotella, Mycoplasma | imbalance; | |
| | | hominis, Gardnerella vaginalis, Mobiluncus, and | BV+ | |
| | | Ureaplasma urealyticum | | |
| Sample | BV+ | Lactobacillus crispatus, Lactobacillus iners, | Flora | Yes |
| 7 | | Atopobium vaginae, Prevotella, Gardnerella | imbalance; | |
| | | vaginalis, Mobiluncus, and Ureaplasma urealyticum | BV+ | |
| Sample | BV+ | Lactobacillus crispatus, Lactobacillus iners, | Flora | Yes |
| 8 | | Atopobium vaginae, Prevotella, Mycoplasma | imbalance; | |
| | | hominis, Gardnerella vaginalis, Mobiluncus, | BV+ | |
| | | Ureaplasma urealyticum, Trichomonas vaginalis, | | |
| | | and other high-risk HPV types | | |
| Sample | BV+ | Lactobacillus crispatus, Lactobacillus iners, | Flora | Yes |
| 9 | | Atopobium vaginae, Prevotella, Mycoplasma | imbalance; | |
| | | hominis, Gardnerella vaginalis, Mobiluncus, | BV+ | |
| | | Ureaplasma urealyticum, Trichomonas vaginalis, | | |
| | | and other high-risk HPV types | | |
| Sample | BV+ | Lactobacillus crispatus, Lactobacillus iners, | Flora | Yes |
| 10 | | Atopobium vaginae, Prevotella, Gardnerella | imbalance; | |
| | | vaginalis, Mobiluncus, Ureaplasma urealyticum, and | BV+ | |
| | | Trichomonas vaginalis | | |
| Sample | VVC+ | Lactobacillus crispatus, Lactobacillus gasseri, | Normal flora, | Yes |
| 11 | | Lactobacillus iners, Candida albicans, Candida spp., | CSTI; VVC+ | |
| | | and Trichomonas vaginalis | | |
| Sample | VVC+ | Lactobacillus crispatus, Lactobacillus jensenii, | Decreased | Yes |
| 12 | | Lactobacillus gasseri, Lactobacillus iners, | flora | |
| | | Atopobium vaginae, Prevotella, Mycoplasma | function, | |
| | | hominis, Gardnerella vaginalis, Bacteroides, | CSTIII; | |
| | | Candida albicans, Candida spp., Ureaplasma | VVC+ | |
| | | urealyticum, and Trichomonas vaginalis | | |
| Sample | VVC+ | Lactobacillus crispatus, Lactobacillus iners, | Decreased | Yes |
| 13 | | Prevotella, Mobiluncus, Candida spp., Ureaplasma | flora | |
| | | urealyticum, and Trichomonas vaginalis | function, | |
| | | | CSTIII; | |
| | | | VVC+ | |
| Sample | VVC+ | Lactobacillus crispatus, Lactobacillus jensenii, | Decreased | Yes |
| 14 | | Lactobacillus gasseri, Lactobacillus iners, | flora | |
| | | Atopobium vaginae, Prevotella, Gardnerella | function, | |
| | | vaginalis, Mobiluncus, Candida albicans, Candida | CSTIII; | |
| | | spp., and Trichomonas vaginalis | VVC+ | |
| Sample | VVC+ | Lactobacillus crispatus, Prevotella, Gardnerella | Normal flora, | Yes |
| 15 | | vaginalis, Candida spp., Staphylococcus aureus, and | CSTI; VVC+ | |
| | | Trichomonas vaginalis | | |
| Sample | VVC+ | Lactobacillus crispatus, Lactobacillus gasseri, | Decreased | Yes |
| 16 | | Lactobacillus iners, Atopobium vaginae, Prevotella, | flora | |
| | | Mycoplasma hominis, Gardnerella vaginalis, | function, | |
| | | Candida albicans, Candida spp., Ureaplasma | CSTIII; | |
| | | urealyticum, and Trichomonas vaginalis | VVC+ | |
| Sample | VVC+ | Lactobacillus crispatus, Lactobacillus gasseri, | Decreased | Yes |
| 17 | | Lactobacillus iners, Atopobium vaginae, Prevotella, | flora | |
| | | Mycoplasma hominis, Gardnerella vaginalis, | function, | |
| | | Mobiluncus, Candida albicans, Candida spp., | CSTIII; | |
| | | Ureaplasma urealyticum, and Trichomonas vaginalis | VVC+ | |
| Sample | TV+ | Lactobacillus crispatus, Lactobacillus gasseri, | Decreased | Yes |
| 18 | | Lactobacillus iners, Atopobium vaginae, Prevotella, | flora | |
| | | Mycoplasma hominis, Gardnerella vaginalis, | function, | |
| | | Mobiluncus, Chlamydia trachomatis, Ureaplasma | CSTIII; TV+ | |
| | | urealyticum, Trichomonas vaginalis, human | | |
| | | papillomavirus type 16, and other high-risk HPV | | |
| | | types | | |
| Sample | TV+ | Lactobacillus crispatus, Lactobacillus jensenii, | Normal flora, | Yes |
| 19 | | Lactobacillus gasseri, Lactobacillus iners, | CSTII; TV+ | |
| | | Atopobium vaginae, Prevotella, Mycoplasma | | |
| | | genitalium, Mobiluncus, Candida albicans, | | |
| | | Ureaplasma urealyticum, and Trichomonas vaginalis | | |
| Sample | TV+ | Lactobacillus crispatus, Lactobacillus iners, | Normal flora, | Yes |
| 20 | | Atopobium vaginae, Prevotella, Mycoplasma | CSTI; TV+ | |
| | | genitalium, Mycoplasma hominis, Gardnerella | | |
| | | vaginalis, Candida albicans, Candida spp., | | |
| | | Ureaplasma urealyticum, Trichomonas vaginalis, | | |
| | | and Group B Streptococcus | | |
| Sample | Normal | Lactobacillus crispatus and Mobiluncus | Normal flora, | Yes |
| 21 | | | CSTI; | |
| | | | Normal | |
| Sample | Normal | Lactobacillus crispatus and Mobiluncus | Normal flora, | Yes |
| 22 | | | CSTI; | |
| | | | Normal | |
| Sample | Normal | Lactobacillus crispatus, Lactobacillus jensenii, | Normal flora, | Yes |
| 23 | | Prevotella, and Mobiluncus | CSTI; | |
| | | | Normal | |
| Sample | Normal | Lactobacillus crispatus, Lactobacillus jensenii, | Normal flora, | Yes |
| 24 | | Lactobacillus iners, Prevotella, and Mobiluncus | CSTI; | |
| | | | Normal | |
| Sample | Normal | Lactobacillus crispatus, Lactobacillus jensenii, | Normal flora, | Yes |
| 25 | | Lactobacillus iners, and Prevotella | CSTV; | |
| | | | Normal | |

As can be seen from Table 13, the results of the microecological evaluation method provided in this example are consistent with those of the gold standard method. Moreover, compared with the gold standard, the method provided in this example can also provide more detailed and accurate microecological flora evaluation results, which is conducive to direct development of more targeted diagnosis and treatment plans by doctors.

2. Comparison with the tNGS Method

Seven clinical samples were collected from a hospital, and the tNGS method and the combination of the present invention were used for simultaneous detection of the samples to verify the accuracy of the combination of the present invention. The results are shown in Table 14 below.

TABLE 14
Comparative results between the combination and the tNGS Method

| | tNGS result | | Detection result of the present invention | | | |
|---|---|---|---|---|---|---|
| Sample | Detected | Reads | Detected | | Concentration | Consistent |
| number | microorganism | count | microorganism | Ct value | (copies/mL) | (Yes/No) |
| Sample | Gardnerella | 30712 | Gardnerella | 23.13 | 1.36E+06 | Yes |
| 26 | Megasphaera | 30031 | Megasphaera | 22.84 | 1.90E+06 | |
| | Prevotella | 22238 | Prevotella | 20.32 | 1.01E+07 | |
| | Atopobium | 19863 | Atopobium | 18.00 | 6.39E+07 | |
| | vaginae | | vaginae | | | |
| | Mobiluncus | 7344 | Mobiluncus | 16.90 | 1.46E+07 | |
| | Lactobacillus | 2529 | Lactobacillus | 24.39 | 8.94E+05 | |
| | iners | | iners | | | |
| Sample | Gardnerella | 41121 | Gardnerella | 23.31 | 1.21E+06 | Yes |
| 27 | Megasphaera | 9485 | Megasphaera | 25.43 | 3.35E+05 | |
| | Prevotella | 12264 | Prevotella | 27.10 | 1.31E+05 | |
| | Atopobium | 49512 | Atopobium | 20.47 | 1.18E+07 | |
| | vaginae | | vaginae | | | |
| | Lactobacillus | 46769 | Lactobacillus | 22.90 | 2.63E+06 | |
| | iners | | iners | | | |
| Sample | Lactobacillus | 271181 | Lactobacillus | 15.90 | 1.08E+08 | Yes |
| 28 | crispatus | | crispatus | | | |
| Sample | Lactobacillus | 306322 | Lactobacillus | 22.28 | 1.36E+06 | Yes |
| 29 | gasseri | | gasseri | | | |
| Sample | Lactobacillus | 199000 | Lactobacillus | 15.94 | 4.09E+08 | Yes |
| 30 | iners | | iners | | | |
| Sample | Lactobacillus | 160083 | Lactobacillus | 15.64 | 1.30E+08 | Yes |
| 31 | crispatus | | crispatus | | | |
| | Lactobacillus | 6057 | Lactobacillus | 22.27 | 1.92E+06 | |
| | jensenii | | jensenii | | | |
| | Lactobacillus | 70633 | Lactobacillus | 18.20 | 7.94E+07 | |
| | iners | | iners | | | |
| | Gardnerella | 2754 | Gardnerella | 29.11 | 2.78E+04 | |
| Sample | Gardnerella | 65154 | Gardnerella | 21.75 | 3.33E+06 | Yes |
| 32 | Streptococcus | 171293 | Streptococcus | 16.67 | 5.68E+08 | Yes |
| | anginosus | | anginosus | | | |

As shown by the results in Table 14, the results of the present invention are consistent with those of the tNGS method. Furthermore, compared with the tNGS method, the method provided in this example has obvious advantages, such as convenient operation, low costs, coverage of major genital tract microorganisms, and capability of quantifying each microorganism.

The above results indicate that according to the present invention, by detecting up to 50 different types and subtypes of genital tract microorganisms rapidly, synchronously and quantitatively, and combining with microorganisms provided and the microbial evaluation method provided by the present invention, evaluation results that are completely consistent with both the gold standard and the tNGS method can be quickly provided, making the microbial evaluation process simpler, more convenient, and efficient.

Although the present invention is disclosed as above, it is not limited thereto. Any person skilled in the art can make various modifications and changes without departing from the spirit and scope of the present invention. Therefore, the scope of protection of the present invention shall be subject to the scope defined by the claims.

Sequence Listing
Upstream primer for Lactobacillus crispatus
SEQ ID NO: 1
CTACGTAAAGAAGTCGGCATGGT
Downstream primer for Lactobacillus crispatus
SEQ ID NO: 2
AAGCCACATTGTCATATACGGAAA
Probe for Lactobacillus crispatus
SEQ ID NO: 3
TCAACAGCCAACCCCATTCCCG
Upstream primer for Lactobacillus jensenii
SEQ ID NO: 4
TAATGTAGTTTATGGTTTGCGTC
Downstream primer for Lactobacillus jensenii
SEQ ID NO: 5
CTCATCCCATAAAGCAGCC
Probe for Lactobacillus jensenii
SEQ ID NO: 6
AGATATTTTAGATCAAAGAGTGGAAGAAAG
Upstream primer for Lactobacillus gasseri
SEQ ID NO: 7
TGGGTCAAGCAAGAACATTG
Downstream primer for Lactobacillus gasseri
SEQ ID NO: 8
CTTAGCTACTAATGATTCCAATTGA
Probe for Lactobacillus gasseri
SEQ ID NO: 9
ACAGTAATTCCGTTCTTAACTACTTTTTTAG
Upstream primer for Lactobacillus iners
SEQ ID NO: 10
GATGGAAGTTGCTATTTTGGAAAA
Downstream primer for Lactobacillus iners
SEQ ID NO: 11
TGGTCGTGACTTACCTAAACG
Probe for Lactobacillus iners
SEQ ID NO: 12
AACGCCTCTAGAAGAAGCACAAGCG
Upstream primer for Atopobium vaginae
SEQ ID NO: 13
CGAGGTTAATACCGGATACTCCA
Downstream primer for Atopobium vaginae
SEQ ID NO: 14
TCTCAACCCGGCTACCCA
Probe for Atopobium vaginae
SEQ ID NO: 15
TCCTTTGCCGCCGGAGCTT
Upstream primer for Prevotella
SEQ ID NO: 16
CGATCAGTAGGGGTTCTGAG
Downstream primer for Prevotella
SEQ ID NO: 17
TACTTGGCTGGTTCAGGCT
Probe for Prevotella
SEQ ID NO: 18
AGGAGTTTGGACCGTGTCTCAG
Upstream primer for Mycoplasma genitalium
SEQ ID NO: 19
TGCAGCAACTAACATGACC
Downstream primer for Mycoplasma genitalium
SEQ ID NO: 20
TTAATCGTTTGACTAACACCCC
Probe for Mycoplasma genitalium
SEQ ID NO: 21
CTGCGAAGAAGCTGTTCCCT
Upstream primer for Mycoplasma hominis
SEQ ID NO: 22
GGCTCGCTTTGGATACTAGCA
Downstream primer for Mycoplasma hominis
SEQ ID NO: 23
CTTTCGTCCCTCAGCGTCA
Probe for Mycoplasma hominis
SEQ ID NO: 24
CCAGTAAGCTGCCTTCGCCTT
Upstream primer for Gardnerella vaginalis
SEQ ID NO: 25
GCAACTATTTCTGCAGCAG
Downstream primer for Gardnerella vaginalis
SEQ ID NO: 26
TTGTCTTCAACGGTAACAACA
Probe for Gardnerella vaginalis
SEQ ID NO: 27
CCTGACCAACCTTATCCAAAGCTTC
Upstream primer for Bacteroides
SEQ ID NO: 28
ATGGATAGGGGTTCTGAGAG
Downstream primer for Bacteroides
SEQ ID NO: 29
ATATTCCTCACTGCTGCCT
Probe for Bacteroides
SEQ ID NO: 30
CCACATTGGDACTGAGACACGG
Upstream primer for Mobiluncus
SEQ ID NO: 31
CAGCAGCTTAACTGTTGGTC
Downstream primer for Mobiluncus
SEQ ID NO: 32
TCCTGATATCTGCGCATTC
Probe for Mobiluncus
SEQ ID NO: 33
AGTCACCCCTACCGCACTCAAG
Upstream primer for herpes simplex virus type 2
SEQ ID NO: 34
TAGACCCGACAAGAGACAGGA
Downstream primer for herpes simplex virus type 2
SEQ ID NO: 35
CGGGAAGGGTGGATGGT
Probe for herpes simplex virus type 2
SEQ ID NO: 36
CATCCTCCGCGGGGGTATAA
Upstream primer for Candida albicans
SEQ ID NO: 37
CAGCAACAGCAGCAAGAA
Downstream primer for Candida albicans
SEQ ID NO: 38
CTGGCATTGACATCTGTTTG
Probe for Candida albicans
SEQ ID NO: 39
AGATGCTGAAGGACAACAACAACAACA
Upstream primer for Candida spp.
SEQ ID NO: 40
CGAAACTCCAAARTACCACGTTACC
Downstream primer for Candida spp.
SEQ ID NO: 41
CACCAGCAATAATCAARATAGCACA
Probe for Candida spp.
SEQ ID NO: 42
TGCTCCAGGTCACAGAGATTTCATCAAG
Upstream primer for Escherichia coli
SEQ ID NO: 43
ATGTCGCGCAAGACTGTAAC
Downstream primer for Escherichia coli
SEQ ID NO: 44
CAGTTGCAACCACCTGTTGA
Probe for Escherichia coli
SEQ ID NO: 45
TGGCCACCACCTGCCAGTCA
Upstream primer for Staphylococcus aureus
SEQ ID NO: 46
CTTATAGGGATGGCTATCAGTAAT
Downstream primer for Staphylococcus aureus
SEQ ID NO: 47
CCGTTATCTGTTTGTGATGC
Probe for Staphylococcus aureus
SEQ ID NO: 48
TGCTGAGCTACTTAGACTTGAAACTAC
Upstream primer for Neisseria gonorrhoeae
SEQ ID NO: 49
AGTCAGTGATTTCGGCTCGTT
Downstream primer for Neisseria gonorrhoeae
SEQ ID NO: 50
GCCTGCCAAGCCGATAAAGGA
Probe for Neisseria gonorrhoeae
SEQ ID NO: 51
AGCTGCCAAACAGCCTTCAG
Upstream primer for Chlamydia trachomatis
SEQ ID NO: 52
CAATGAAAATCCATTGCGT
Downstream primer for Chlamydia trachomatis
SEQ ID NO: 53
TCTGACTGTGAGAATATAT
Probe for Chlamydia trachomatis
SEQ ID NO: 54
TAGAGGCCGGTCTATTTATGA
Upstream primer for Ureaplasma urealyticum
SEQ ID NO: 55
GTGCAAAACCTTACTATGTTGT
Downstream primer for Ureaplasma urealyticum
SEQ ID NO: 56
TGAACGTCCATATGTTCCAAA
Probe for Ureaplasma urealyticum
SEQ ID NO: 57
TGGGTGTAATTGCTCGTTGTGTAG
Upstream primer for Trichomonas vaginalis
SEQ ID NO: 58
CAACAGCCTTCCGTGAACTCT
Downstream primer for Trichomonas vaginalis
SEQ ID NO: 59
CGAGGCCTTCGTTGACGTA
Probe for Trichomonas vaginalis
SEQ ID NO: 60
CCAGAAGATGTACGCTCGCCGTGCT
Upstream primer for HPV type 16
SEQ ID NO: 61
GAATTCYACTATTTTGGAGGAC
Downstream primer for HPV type 16
SEQ ID NO: 62
TTGACAAGCAATTGCCTGG
Probe for HPV type 16
SEQ ID NO: 63
CCAGGAGGCACACTAGAAGATAC
Upstream primer for HPV type 18
SEQ ID NO: 64
TGACACTGTGCCTCAATCCT
Downstream primer for HPV type 18
SEQ ID NO: 65
ACAACTGGGAGTCAGAGGTAAC
Probe for HPV type 18
SEQ ID NO: 66
AGGTGAAGCACGCATACCTGTGCC
Upstream primer for HPV type 31
SEQ ID NO: 67
ATTACCCGACAGCTCAGAT
Upstream primer for HPV type 35
SEQ ID NO: 68
CCGAGGCAACTGACCTA
Upstream primer for HPV type 53
SEQ ID NO: 69
CTGAGATTGACCTGCAATGC
Upstream primer for HPV type 56 and HPV type 66
SEQ ID NO: 70
TACARTGCAATGAGCAATTGG
Upstream primer for HPV type 39 and HPV type 68
SEQ ID NO: 71
CCACCKTGCAGGAAATTGTGTTA
Upstream primer for HPV type 33 and HPV type 58
SEQ ID NO: 72
TTTATAACCCTGATACACAACG
Upstream primer for HPV type 51 and HPV type 82
SEQ ID NO: 73
AAATAGCGGGACGYTGGAC
Upstream primer for HPV type 26
SEQ ID NO: 74
CAACAGGGACAATGTTTCAGTT
Upstream primer for HPV type 73
SEQ ID NO: 75
CCTGATAAGGAGCGCCTAGTATG
Upstream primer for HPV type 45
SEQ ID NO: 76
GCCATCCCATACTCCTATG
Upstream primer for HPV type 52
SEQ ID NO: 77
GAAACCAGTAACAAATATGCTGGT
Upstream primer for HPV type 59
SEQ ID NO: 78
CTCATGTAGCATCTGCTGTTG
Downstream primer for HPV type 31
SEQ ID NO: 79
CACTGACAACAAAAGGTAACG
Downstream primer for HPV type 35
SEQ ID NO: 80
GTAGTGTCGCCTYACATTTA
Downstream primer for HPV type 53 and HPV types 56 and 66
SEQ ID NO: 81
GTCTAGCTTGCTGTGGC
Downstream primer for HPV type 39 and HPV type 68
SEQ ID NO: 82
TAACTGCATGGTCGGGTTCATC
Downstream primer for HPV type 33 and HPV type 58
SEQ ID NO: 83
GGATGACCACTTATGCCAA
Downstream primer for HPV type 51 and HPV type 82
SEQ ID NO: 84
TGCTCGTAGCATTGCAAGTCAA
Downstream primer for HPV type 26
SEQ ID NO: 85
CCACGTTGTGTCTGTGT
Downstream primer for HPV type 73
SEQ ID NO: 86
TGCCACTAGTACCTATACCTAAGGG
Downstream primer for HPV type 45
SEQ ID NO: 87
TTGTGTGCCATGAATACCT
Downstream primer for HPV type 52
SEQ ID NO: 88
AGGAGGTTTGCATCCTAAAATG
Downstream primer for HPV type 59
SEQ ID NO: 89
TAGGCTTACAAGCAGTGC
Probe for HPV type 31 and HPV type 35
SEQ ID NO: 90
TCCAGCTGGACAAGCAAAACC
Probe for HPV type 53, HPV type 56, and HPV type 66
SEQ ID NO: 91
CATCCTCATCCTCATCCTCTGAGCTGT
Probe for HPV type 39 and HPV type 68
SEQ ID NO: 92
CTCGTGACATACAAGGTCAACCGGCT
Probe for HPV type 33 and HPV type 58
SEQ ID NO: 93
CCTATTTCAAGGCCTACACATGCCC
Probe for HPV type 51 and HPV type 82
SEQ ID NO: 94
AATTGTGGTACATTACCACGCATGGC
Probe for HPV type 26
SEQ ID NO: 95
TAGGTTGTACACCGCCCTTGGGAGAGCAC
Probe for HPV type 73
SEQ ID NO: 96
CGTCCAACCTCCACACCAGAACAGGC
Probe for HPV type 45
SEQ ID NO: 97
CATCTCCTACCAATGCTKCCACC
Probe for HPV type 52
SEQ ID NO: 98
CATTCYCTATTATCTATACCAGG
Probe for HPV type 59
SEQ ID NO: 99
CTCCAATGGCAGGTACACAGCC
Upstream primer for Group B Streptococcus
SEQ ID NO: 100
TAGAAGCCTTAACAGATGTGA
Downstream primer for Group B Streptococcus
SEQ ID NO: 101
GTTCTAATGCCTTTACATCGTT
Probe for Group B Streptococcus
SEQ ID NO: 102
AAGCAAATGGATCTAAAATGCGAA
Upstream primer for Megasphaera
SEQ ID NO: 103
CTGAATATGCTATCGCCGGAA
Downstream primer for Megasphaera
SEQ ID NO: 104
AATGTGGCCGTTCATCC
Probe for Megasphaera
SEQ ID NO: 105
CAGACCGGCTACTGATCATTGCCTT
Upstream primer for BVAB2
SEQ ID NO: 106
ATGCGTAGATATTAGGAGGAACACCG
Downstream primer for BVAB2
SEQ ID NO: 107
GGGTCGATACCTCCTACAYC
Probe for BVAB2
SEQ ID NO: 108
ACCCTGGTAGTCCACGCCGTAAACGA
Upstream primer for Treponema pallidum
SEQ ID NO: 109
GGTACTTCCCGGTTCGTTGTATC
Downstream primer for Treponema pallidum
SEQ ID NO: 110
ACCCAAACGCAGCTTGAATC
Probe for Treponema pallidum
SEQ ID NO: 111
CGATGATCGCAGTGCTGCCCAC
Upstream primer for herpes simplex virus type 1
SEQ ID NO: 112
GGCCTGGCTATCCGGAGA
Downstream primer for herpes simplex virus type 1
SEQ ID NO: 113
GCGCAGAGACATCGCGA
Probe for herpes simplex virus type 1
SEQ ID NO: 114
ACGACTTGGCGTTCTG
Upstream primer for Streptococcus anginosus
SEQ ID NO: 115
TTCTTCGGAACATCGGTGACAG
Downstream primer for Streptococcus anginosus
SEQ ID NO: 116
TTGTACCAGCCATTGTAGCAC
Probe for Streptococcus anginosus
SEQ ID NO: 117
CACGAGCTGACGACAACCATGCACCAC
Upstream primer for Enterococcus faecalis
SEQ ID NO: 118
TTAATGGCTGCAGTTGACGAAT
Downstream primer for Enterococcus faecalis
SEQ ID NO: 119
CGTCTTCGACTGGCATCATG
Probe for Enterococcus faecalis
SEQ ID NO: 120
CCCAACTCCAGAACGTGATACTGACAAACC
Upstream primer for Klebsiella pneumoniae
SEQ ID NO: 121
CGATATGTTCCCGGAATTCG
Downstream primer for Klebsiella pneumoniae
SEQ ID NO: 122
GCGCGCTTGGTCATAAAGTT
Probe for Klebsiella pneumoniae
SEQ ID NO: 123
CGGCGATTCCTCTGCCCAGACC
Upstream primer for Group A Streptococcus
SEQ ID NO: 124
AGATAATTCTTTGTCAATTTGTGMT
Downstream primer for Group A Streptococcus
SEQ ID NO: 125
TGAAAGAAAACTTTGGCTACAACC
Probe for Group A Streptococcus
SEQ ID NO: 126
ATCTTGTTTGCTAAAGTCGCTACGG
Upstream primer for internal standard RNase P
SEQ ID NO: 127
AATGTGGCGATTGACCGAG
Downstream primer for internal standard RNase P
SEQ ID NO: 128
TTGAGGGCACTGGAAATTGTA
Probe for internal standard RNase P
SEQ ID NO: 129
CCCTGCTATCAAAGACTCCACAATG

1. A kit for detecting a genital tract microorganism rapidly, synchronously and quantitatively, comprising any one or more of combinations of upstream primers, downstream primers, and probes for amplifying 32 groups of genital tract microorganisms as shown in the table below:

| Serial | Detected | | Downstream | |
|---|---|---|---|---|
| number | microorganism | Upstream primer | primer | Probe |
| 1 | Lactobacillus | SEQ ID NO. 1 | SEQ ID NO. 2 | SEQ ID NO. 3 |
| | crispatus | | | |
| 2 | Lactobacillus | SEQ ID NO. 4 | SEQ ID NO. 5 | SEQ ID NO. 6 |
| | jensenii | | | |
| 3 | Lactobacillus | SEQ ID NO. 7 | SEQ ID NO. 8 | SEQ ID NO. 9 |
| | gasseri | | | |
| 4 | Lactobacillus iners | SEQ ID NO. 10 | SEQ ID NO. 11 | SEQ ID NO. 12 |
| 5 | Atopobium vaginae | SEQ ID NO. 13 | SEQ ID NO. 14 | SEQ ID NO. 15 |
| 6 | Prevotella | SEQ ID NO. 16 | SEQ ID NO. 17 | SEQ ID NO. 18 |
| 7 | Mycoplasma | SEQ ID NO. 19 | SEQ ID NO. 20 | SEQ ID NO. 21 |
| | genitalium | | | |
| 8 | Mycoplasma hominis | SEQ ID NO. 22 | SEQ ID NO. 23 | SEQ ID NO. 24 |
| 9 | Gardnerella | SEQ ID NO. 25 | SEQ ID NO. 26 | SEQ ID NO. 27 |
| | vaginalis | | | |
| 10 | Bacteroides | SEQ ID NO. 28 | SEQ ID NO. 29 | SEQ ID NO. 30 |
| 11 | Mobiluncus | SEQ ID NO. 31 | SEQ ID NO. 32 | SEQ ID NO. 33 |
| 12 | Herpes simplex virus | SEQ ID NO. 34 | SEQ ID NO. 35 | SEQ ID NO. 36 |
| | type 2 | | | |
| 13 | Candida albicans | SEQ ID NO. 37 | SEQ ID NO. 38 | SEQ ID NO. 39 |
| 14 | Candida spp. | SEQ ID NO. 40 | SEQ ID NO. 41 | SEQ ID NO. 42 |
| 15 | Escherichia coli | SEQ ID NO. 43 | SEQ ID NO. 44 | SEQ ID NO. 45 |
| 16 | Staphylococcus | SEQ ID NO. 46 | SEQ ID NO. 47 | SEQ ID NO. 48 |
| | aureus | | | |
| 17 | Neisseria | SEQ ID NO. 49 | SEQ ID NO. 50 | SEQ ID NO. 51 |
| | gonorrhoeae | | | |
| 18 | Chlamydia | SEQ ID NO. 52 | SEQ ID NO. 53 | SEQ ID NO. 54 |
| | trachomatis | | | |
| 19 | Ureaplasma | SEQ ID NO. 55 | SEQ ID NO. 56 | SEQ ID NO. 57 |
| | urealyticum | | | |
| 20 | Trichomonas | SEQ ID NO. 58 | SEQ ID NO. 59 | SEQ ID NO. 60 |
| | vaginalis | | | |
| 21 | HPV type 16 | SEQ ID NO. 61 | SEQ ID NO. 62 | SEQ ID NO. 63 |
| 22 | HPV type 18 | SEQ ID NO. 64 | SEQ ID NO. 65 | SEQ ID NO. 66 |
| 23 | HPV type 31 | SEQ ID NO. 67 | SEQ ID NO. 79 | SEQ ID NO. 90 |
| 24 | Group B | SEQ ID NO. 100 | SEQ ID NO. 101 | SEQ ID NO. 102 |
| | Streptococcus | | | |
| 25 | Megasphaera | SEQ ID NO. 103 | SEQ ID NO. 104 | SEQ ID NO. 105 |
| 26 | BVAB2 | SEQ ID NO. 106 | SEQ ID NO. 107 | SEQ ID NO. 108 |
| 27 | Treponema pallidum | SEQ ID NO. 109 | SEQ ID NO. 110 | SEQ ID NO. 111 |
| 28 | Herpes simplex virus | SEQ ID NO. 112 | SEQ ID NO. 113 | SEQ ID NO. 114 |
| | type 1 | | | |
| 29 | Streptococcus | SEQ ID NO. 115 | SEQ ID NO. 116 | SEQ ID NO. 117 |
| | anginosus | | | |
| 30 | Enterococcus | SEQ ID NO. 118 | SEQ ID NO. 119 | SEQ ID NO. 120 |
| | faecalis | | | |
| 31 | Klebsiella | SEQ ID NO. 121 | SEQ ID NO. 122 | SEQ ID NO. 123 |
| | pneumoniae | | | |
| 32 | Group A | SEQ ID NO. 124 | SEQ ID NO. 125 | SEQ ID NO. 126 |
| | Streptococcus | | | |

2. The kit according to claim 1, comprising eight groups of primer-probe combinations as shown in the table below, wherein each group is capable of achieving synchronous detection of genital tract microorganisms within the group:

| | Detected | | Downstream | |
|---|---|---|---|---|
| Group | microorganism | Upstream primer | primer | Probe |
| First | Lactobacillus | SEQ ID NO. 1 | SEQ ID NO. 2 | SEQ ID NO. 3 |
| group | crispatus | | | |
| | Lactobacillus | SEQ ID NO. 4 | SEQ ID NO. 5 | SEQ ID NO. 6 |
| | jensenii | | | |
| | Lactobacillus | SEQ ID NO. 7 | SEQ ID NO. 8 | SEQ ID NO. 9 |
| | gasseri | | | |
| | Lactobacillus iners | SEQ ID NO. 10 | SEQ ID NO. 11 | SEQ ID NO. 12 |
| Second | Atopobium vaginae | SEQ ID NO. 13 | SEQ ID NO. 14 | SEQ ID NO. 15 |
| group | Prevotella | SEQ ID NO. 16 | SEQ ID NO. 17 | SEQ ID NO. 18 |
| | Mycoplasma | SEQ ID NO. 19 | SEQ ID NO. 20 | SEQ ID NO. 21 |
| | genitalium | | | |
| | Mycoplasma hominis | SEQ ID NO. 22 | SEQ ID NO. 23 | SEQ ID NO. 24 |
| Third | Gardnerella | SEQ ID NO. 25 | SEQ ID NO. 26 | SEQ ID NO. 27 |
| group | vaginalis | | | |
| | Bacteroides | SEQ ID NO. 28 | SEQ ID NO. 29 | SEQ ID NO. 30 |
| | Mobiluncus | SEQ ID NO. 31 | SEQ ID NO. 32 | SEQ ID NO. 33 |
| | Herpes simplex virus | SEQ ID NO. 34 | SEQ ID NO. 35 | SEQ ID NO. 36 |
| | type 2 | | | |
| Fourth | Candida albicans | SEQ ID NO. 37 | SEQ ID NO. 38 | SEQ ID NO. 39 |
| group | Candida spp. | SEQ ID NO. 40 | SEQ ID NO. 41 | SEQ ID NO. 42 |
| | Escherichia coli | SEQ ID NO. 43 | SEQ ID NO. 44 | SEQ ID NO. 45 |
| | Staphylococcus | SEQ ID NO. 46 | SEQ ID NO. 47 | SEQ ID NO. 48 |
| | aureus | | | |
| Fifth | Neisseria | SEQ ID NO. 49 | SEQ ID NO. 50 | SEQ ID NO. 51 |
| group | gonorrhoeae | | | |
| | Chlamydia | SEQ ID NO. 52 | SEQ ID NO. 53 | SEQ ID NO. 54 |
| | trachomatis | | | |
| | Ureaplasma | SEQ ID NO. 55 | SEQ ID NO. 56 | SEQ ID NO. 57 |
| | urealyticum | | | |
| | Trichomonas | SEQ ID NO. 58 | SEQ ID NO. 59 | SEQ ID NO. 60 |
| | vaginalis | | | |
| Sixth | HPV type 16 | SEQ ID NO. 61 | SEQ ID NO. 62 | SEQ ID NO. 63 |
| group | HPV type 18 | SEQ ID NO. 64 | SEQ ID NO. 65 | SEQ ID NO. 66 |
| | HPV type 31 | SEQ ID NO. 67 | SEQ ID NO. 79 | SEQ ID NO. 90 |
| | Group B | SEQ ID NO. 100 | SEQ ID NO. 101 | SEQ ID NO. 102 |
| | Streptococcus | | | |
| Seventh | Megasphaera | SEQ ID NO. 103 | SEQ ID NO. 104 | SEQ ID NO. 105 |
| group | BVAB2 | SEQ ID NO. 106 | SEQ ID NO. 107 | SEQ ID NO. 108 |
| | Treponema pallidum | SEQ ID NO. 109 | SEQ ID NO. 110 | SEQ ID NO. 111 |
| | Herpes simplex virus | SEQ ID NO. 112 | SEQ ID NO. 113 | SEQ ID NO. 114 |
| | type 1 | | | |
| Eighth | Streptococcus | SEQ ID NO. 115 | SEQ ID NO. 116 | SEQ ID NO. 117 |
| group | anginosus | | | |
| | Enterococcus | SEQ ID NO. 118 | SEQ ID NO. 119 | SEQ ID NO. 120 |
| | faecalis | | | |
| | Klebsiella | SEQ ID NO. 121 | SEQ ID NO. 122 | SEQ ID NO. 123 |
| | pneumoniae | | | |
| | Group A | SEQ ID NO. 124 | SEQ ID NO. 125 | SEQ ID NO. 126 |
| | Streptococcus | | | |

3. The kit according to claim 2, wherein the sixth group further comprises any one or more of 11 primer-probe combinations as shown in the table below:

| | Detected | Upstream | Downstream | |
|---|---|---|---|---|
| Group | microorganism | primer | primer | Probe |
| a1 | HPV type 35 | SEQ ID NO. | SEQ ID NO. 80 | SEQ ID NO. 90 |
| | | 68 | | |
| a 2 | HPV type 53 | SEQ ID NO. | SEQ ID NO. 81 | SEQ ID NO. 91 |
| | | 69 | | |
| a 3 | HPV types 56 | SEQ ID NO. | SEQ ID NO. 81 | SEQ ID NO. 91 |
| | and 66 | 70 | | |
| a 4 | HPV types 39 | SEQ ID NO. | SEQ ID NO. 82 | SEQ ID NO. 92 |
| | and 68 | 71 | | |
| a 5 | HPV types 33 | SEQ ID NO. | SEQ ID NO. 83 | SEQ ID NO. 93 |
| | and 58 | 72 | | |
| a 6 | HPV types 51 | SEQ ID NO. | SEQ ID NO. 84 | SEQ ID NO. 94 |
| | and 82 | 73 | | |
| a 7 | HPV type 26 | SEQ ID NO. | SEQ ID NO. 85 | SEQ ID NO. 95 |
| | | 74 | | |
| a 8 | HPV type 73 | SEQ ID NO. | SEQ ID NO. 86 | SEQ ID NO. 96 |
| | | 75 | | |
| a 9 | HPV type 45 | SEQ ID NO. | SEQ ID NO. 87 | SEQ ID NO. 97 |
| | | 76 | | |
| a 10 | HPV type 52 | SEQ ID NO. | SEQ ID NO. 88 | SEQ ID NO. 98 |
| | | 77 | | |
| a 11 | HPV type 59 | SEQ ID NO. | SEQ ID NO. 89 | SEQ ID NO. 99 |
| | | 78 | | |

4. The kit according to claim 3, wherein each group further comprises a primer-probe combination of an internal standard.

5. The kit according to claim 4, wherein the internal standard is an RNase P gene, with an upstream primer sequence as shown in SEQ ID NO.127, a downstream primer sequence as shown in SEQ ID NO.128, and a probe sequence as shown in SEQ ID NO.129.

6. The kit according to claim 5, wherein the primer-probe combinations and an internal standard probe are respectively labeled at 5′ terminals with different fluorescent groups and at 3′ terminals with quenching groups.

7. The kit according to claim 6, wherein for the specific probe, the fluorescent group labeled at the 5′ terminal is selected from any one or more of FAM, HEX, VIC, JOE, TET, TAMRA, ROX, QUASAR 705, CY3.5, or CY5; and the quenching group is selected from any one or more of BHQ1, BHQ2, BHQ3, TAMRA, DABCYL, and MGB.

8. The kit according to claim 3, further comprising an amplification reaction solution, wherein the amplification reaction solution comprises a Taq DNA polymerase and a UDG enzyme.

9. The kit according to claim 8, wherein contents of the DNA polymerase and UDG enzyme are both 3-10 U/Test; and the PCR buffer solution is a 2×Buffer solution.

10. The kit according to claim 8, wherein the amplification reaction solution further comprises a PCR buffer solution, MgCl₂, dNTPs, and RNase-free water.

11. The kit according to claim 10, wherein a concentration of the MgCl₂ is 0-10 mM; and a concentration of the dNTPs is 0.5-1 mM, with dATP:dTTP:dCTP:dGTP:dUTP=1:1:1:1:1.

12. The kit according to claim 3, further comprising a positive control and a negative control, wherein the positive control is at least one of a culture, a plasmid fragment, or a DNA fragment of the microorganism; and the negative control is at least one of a negative sample and an internal standard plasmid.

13. A microecological evaluation method for detecting a genital tract microorganism rapidly, synchronously and quantitatively, comprising:
adopting the kit according to claim 2 to perform nucleic acid amplification on a microorganism from a genital tract sample and perform nucleic acid detection on an amplification product to obtain types and quantities of microorganisms in a genital tract, thereby performing microecological evaluation and analysis of the genital tract.

14. The microecological evaluation method according to claim 13, comprising performing evaluation and analysis by adopting the following evaluation method, wherein the evaluation method comprises: any one or more of evaluation of genital tract microecology, evaluation of bacterial vaginitis (BV), evaluation of aerobic vaginitis (AV), evaluation of vulvovaginal candidiasis (VVC), evaluation of trichomoniasis vaginalis (TV), evaluation of sexually transmitted infection (STI), and evaluation of HPV.

15. The microecological evaluation method according to claim 14, wherein the evaluation method of genital tract microecology comprises: assessing vaginal microecology according to the quantification results of 4 vaginal lactobacillus groups: Lactobacillus crispatus, Lactobacillus jensenii, Lactobacillus gasseri, and Lactobacillus iners, wherein when Lactobacillus crispatus accounts for a relatively high proportion, a result is “normal flora, CSTI”; when Lactobacillus jensenii accounts for a relatively high proportion, a result is “normal flora, CSTV”; when Lactobacillus gasseri accounts for a relatively high proportion, a result is “normal flora, CSTII”; when Lactobacillus iners accounts for a relatively high proportion, a result is “decreased flora function, CSTIII”; and when a proportion of pathogenic microorganisms of bacterial vaginitis (BV) or aerobic vaginitis (AV) is higher than that of the lactobacillus groups, a result is “flora imbalance.”

16. The microecological evaluation method according to claim 14, wherein the evaluation of bacterial vaginitis (BV) comprises:
obtaining a P value (BV) by dividing a sum of concentrations of Lactobacillus crispatus, Lactobacillus jensenii, Lactobacillus gasseri, and Lactobacillus iners by a sum of concentrations of Gardnerella vaginalis, Prevotella, Mobiluncus, Bacteroides, Atopobium vaginae, Megasphaera, BVAB2, Ureaplasma urealyticum, and Mycoplasma hominis, wherein if the P value (BV)≤1, a result is determined as bacterial vaginitis positive; and if the P value (BV)>1, a result is determined as bacterial vaginitis negative; and/or
the evaluation of aerobic vaginitis (AV) comprises: obtaining a P value (AV) by dividing the sum of concentrations of Lactobacillus crispatus, Lactobacillus jensenii, Lactobacillus gasseri, and Lactobacillus iners by a sum of concentrations of Group A Streptococcus, Group B Streptococcus, Staphylococcus aureus, Escherichia coli, Enterococcus faecalis, Streptococcus anginosus, and Klebsiella pneumoniae,
wherein if the P value (AV)≤1, a result is determined as aerobic vaginitis positive; and
if the P value (AV)>1, a result is determined as aerobic vaginitis negative.

17. The microecological evaluation method according to claim 14, wherein the evaluation of vulvovaginal candidiasis (VVC) comprises: if Candida spp, and/or Candida albicans is detected as positive, a result is determined as VVC positive; and if Candida spp, and/or Candida albicans is detected as negative, a result is determined as VVC negative.

18. The microecological evaluation method according to claim 14, wherein the evaluation of trichomoniasis vaginalis (TV) comprises: if Trichomonas vaginalis is detected as positive, a result is determined as TV positive; and if Trichomonas vaginalis is detected as negative, a result is determined as TV negative.

19. The microecological evaluation method according to claim 14, wherein the evaluation of sexually transmitted infection (STI) comprises: if one or more of Neisseria gonorrhoeae, Treponema pallidum, Chlamydia trachomatis, Mycoplasma genitalium, herpes simplex virus type I, and herpes simplex virus type II are detected as positive, a result is determined as STI positive; and if one or more of Neisseria gonorrhoeae, Treponema pallidum, Chlamydia trachomatis, Mycoplasma genitalium, herpes simplex virus type I, and herpes simplex virus type II are detected as negative, a result is determined as STI negative.

20. The microecological evaluation method according to claim 14, wherein the evaluation of HPV comprises: if any one or more of HPV16, HPV18, and other high-risk HPV types are detected as positive, a result is determined as HPV positive; and if any one or more of HPV16, HPV18, and other high-risk HPV types are detected as negative, a result is determined as HPV negative.
